SEQUENCE LISTING (51 sequences)

SEQ ID NO: 1 - protein, 42 aa, Artificial Sequence
  4-1BB costimulatory domain amino acid sequence
KRGRKKLLYIFKQPFMRPVQTTQEEDGCSCRFPEEEEGGCEL

SEQ ID NO: 2 - DNA, 126 bp, Artificial Sequence
  4-1BB costimulatory domain nucleic acid sequence #1
aaacggggcagaaagaaactcctgtatatattcaaacaaccatttatgagaccagtacaaactactcaagaggaagacggctgtagctgccgatttccagaagaagaagaaggaggatgtgaactg

SEQ ID NO: 3 - DNA, 126 bp, Artificial Sequence
  4-1BB costimulatory domain nucleic acid sequence #2
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 4 - protein, 41 aa, Artificial Sequence
  CD28 costimulatory domain amino acid sequence
RSKRSRLLHSDYMNMTPRRPGPTRKHYQPYAPPRDFAAYRS

SEQ ID NO: 5 - DNA, 123 bp, Artificial Sequence
  CD28 costimulatory domain nucleic acid sequence
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 6 - protein, 41 aa, Artificial Sequence
  CD28(YMFM) costimulatory domain amino acid sequence
RSKRSRLLHSDYMFMTPRRPGPTRKHYQPYAPPRDFAAYRS

SEQ ID NO: 7 - DNA, 123 bp, Artificial Sequence
  CD28(YMFM) costimulatory domain nucleic acid sequence
aggagtaagaggagcaggctcctgcacagtgactacatgttcatgactccccgccgccccgggcccacccgcaagcattaccagccctatgccccaccacgcgacttcgcagcctatcgctcc

SEQ ID NO: 8 - protein, 35 aa, Artificial Sequence
  ICOS costimulatory domain amino acid sequence
TKKKYSSSVHDPNGEYMFMRAVNTAKKSRLTDVTL

SEQ ID NO: 9 - DNA, 105 bp, Artificial Sequence
  ICOS costimulatory domain nucleic acid sequence #1
acaaaaaagaagtattcatccagtgtgcacgaccctaacggtgaatacatgttcatgagagcagtgaacacagccaaaaaatccagactcacagatgtgacccta

SEQ ID NO: 10 - DNA, 105 bp, Artificial Sequence
  ICOS costimulatory domain nucleic acid sequence #2
acaaaaaagaagtattcatccagtgtgcacgaccctaacggtgaatacatgttcatgagagcagtgaacacagccaaaaaatctagactcacagatgtgacccta

SEQ ID NO: 11 - protein, 35 aa, Artificial Sequence
  ICOS(YMNM) costimulatory domain amino acid sequence
TKKKYSSSVHDPNGEYMNMRAVNTAKKSRLTDVTL

SEQ ID NO: 12 - DNA, 105 bp, Artificial Sequence
  ICOS(YMNM) costimulatory domain nucleic acid sequence
acaaaaaagaagtattcatccagtgtgcacgaccctaacggtgaatacatgaacatgagagcagtgaacacagccaaaaaatccagactcacagatgtgacccta

SEQ ID NO: 13 - protein, 117 aa, Artificial Sequence
  CD2 costimulatory domain amino acid sequence
TKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 14 - DNA, 351 bp, Artificial Sequence
  CD2 costimulatory domain nucleic acid sequence
accaaaaggaaaaaacagaggagtcggagaaatgatgaggagctggagacaagagcccacagagtagctactgaagaaaggggccggaagccccaccaaattccagcttcaacccctcagaatccagcaacttcccaacatcctcctccaccacctggtcatcgttcccaggcacctagtcatcgtcccccgcctcctggacaccgtgttcagcaccagcctcagaagaggcctcctgctccgtcgggcacacaagttcaccagcagaaaggcccgcccctccccagacctcgagttcagccaaaacctccccatggggcagcagaaaactcattgtccccttcctctaat

SEQ ID NO: 15 - protein, 48 aa, Artificial Sequence
  CD27 costimulatory domain amino acid sequence
QRRKYRSNKGESPVEPAEPCRYSCPREEEGSTIPIQEDYRKPEPACSP

SEQ ID NO: 16 - DNA, 144 bp, Artificial Sequence
  CD27 costimulatory domain nucleic acid sequence
caacgaaggaaatatagatcaaacaaaggagaaagtcctgtggagcctgcagagccttgtcgttacagctgccccagggaggaggagggcagcaccatccccatccaggaggattaccgaaaaccggagcctgcctgctccccc

SEQ ID NO: 17 - protein, 42 aa, Artificial Sequence
  OX40 costimulatory domain amino acid sequence
ALYLLRRDQRLPPDAHKPPGGGSFRTPIQEEQADAHSTLAKI

SEQ ID NO: 18 - DNA, 126 bp, Artificial Sequence
  OX40 costimulatory domain nucleic acid sequence
gccctgtacctgctccgcagggaccagaggctgccccccgatgcccacaagccccctgggggaggcagtttcaggacccccatccaagaggagcaggccgacgcccactccaccctggccaagatc

SEQ ID NO: 19 - protein, 112 aa, Artificial Sequence
  CD3 zeta intracellular signaling domain amino acid sequence
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - DNA, 336 bp, Artificial Sequence
  CD3 zeta intracellular signaling domain nucleic acid sequence
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 21 - protein, 112 aa, Artificial Sequence
  CD3 zeta (Q14K) intracellular signaling domain amino acid sequence
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - DNA, 336 bp, Artificial Sequence
  CD3 zeta (Q14K) intracellular signaling domain nucleic acid sequence
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 23 - protein, 24 aa, Artificial Sequence
  CD8 alpha transmembrane domain amino acid sequence
IYIWAPLAGTCGVLLLSLVITLYC

SEQ ID NO: 24 - DNA, 72 bp, Artificial Sequence
  CD8 alpha transmembrane domain nucleic acid sequence
atctacatctgggcgcccttggccgggacttgtggggtccttctcctgtcactggttatcaccctttactgc

SEQ ID NO: 25 - protein, 45 aa, Artificial Sequence
  CD8 alpha hinge domain amino acid sequence
TTTPAPRPPTPAPTIASQPLSLRPEACRPAAGGAVHTRGLDFACD

SEQ ID NO: 26 - DNA, 135 bp, Artificial Sequence
  CD8 alpha hinge domain nucleic acid sequence
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 27 - protein, 27 aa, Artificial Sequence
  CD28 transmembrane domain amino acid sequence
FWVLVVVGGVLACYSLLVTVAFIIFWV

SEQ ID NO: 28 - DNA, 81 bp, Artificial Sequence
  CD28 transmembrane domain nucleic acid sequence
ttttgggtgctggtggtggttggtggagtcctggcttgctatagcttgctagtaacagtggcctttattattttctgggtg

SEQ ID NO: 29 - protein, 4 aa, Artificial Sequence
  hinge region
GGSG

SEQ ID NO: 30 - protein, 5 aa, Artificial Sequence
  hinge region
GGSGG

SEQ ID NO: 31 - protein, 5 aa, Artificial Sequence
  hinge region
GSGSG

SEQ ID NO: 32 - protein, 5 aa, Artificial Sequence
  hinge region
GSGGG

SEQ ID NO: 33 - protein, 5 aa, Artificial Sequence
  hinge region
GGGSG

SEQ ID NO: 34 - protein, 5 aa, Artificial Sequence
  hinge region
GSSSG

SEQ ID NO: 35 - protein, 5 aa, Artificial Sequence
  hinge region
DKTHT

SEQ ID NO: 36 - protein, 4 aa, Artificial Sequence
  hinge region
CPPC

SEQ ID NO: 37 - protein, 15 aa, Artificial Sequence
  hinge region
CPEPKSCDTPPPCPR

SEQ ID NO: 38 - protein, 12 aa, Artificial Sequence
  hinge region
ELKTPLGDTTHT

SEQ ID NO: 39 - protein, 10 aa, Artificial Sequence
  hinge region
KSCDKTHTCP

SEQ ID NO: 40 - protein, 7 aa, Artificial Sequence
  hinge region
KCCVDCP

SEQ ID NO: 41 - protein, 7 aa, Artificial Sequence
  hinge region
KYGPPCP

SEQ ID NO: 42 - protein, 15 aa, Homo sapiens
  MISC_FEATURE (1)..(15): hinge region
EPKSCDKTHTCPPCP

SEQ ID NO: 43 - protein, 12 aa, Homo sapiens
  MISC_FEATURE (1)..(12): hinge region
ERKCCVECPPCP

SEQ ID NO: 44 - protein, 17 aa, Homo sapiens
  MISC_FEATURE (1)..(17): hinge region
ELKTPLGDTTHTCPRCP

SEQ ID NO: 45 - protein, 12 aa, Homo sapiens
  MISC_FEATURE (1)..(12): hinge region
SPNMVPHAHHAQ

SEQ ID NO: 46 - protein, 15 aa, Artificial Sequence
  hinge region
EPKSCDKTYTCPPCP

SEQ ID NO: 47 - protein, 5 aa, Artificial Sequence
  hinge region
GSGGS

SEQ ID NO: 48 - protein, 4 aa, Artificial Sequence
  hinge region
GGGS

SEQ ID NO: 49 - protein, 5 aa, Artificial Sequence
  hinge region
GGGGS

SEQ ID NO: 50 - protein, 15 aa, Artificial Sequence
  linker
GGGGSGGGGSGGGGS

SEQ ID NO: 51 - DNA, 45 bp, Artificial Sequence
  linker
ggtggcggtggctcgggcggtggtgggtcgggtggcggcggatct

(19) United States
(12) Patent Application Publication
(10) Pub. No.: US 2024/0060089 A1
(43) Pub. Date: Feb. 22, 2024

(54) VECTOR-FREE PROCESS FOR MANUFACTURE OF ENGINEERED IMMUNE CELLS

(71) Applicant: Tmunity Therapeutics Inc., Philadelphia, PA (US)

(72) Inventors: Felipe Bedoya, Philadelphia, PA (US); Zoe Zheng, Philadelphia, PA (US)

(73) Assignee: Tmunity Therapeutics Inc., Philadelphia, PA (US)

(21) Appl. No.: 18/022,968

(22) PCT Filed: Aug. 26, 2021
(86) PCT No.: PCT/US2021/047710

§ 371 Date: Feb. 23, 2023

Related U.S. Application Data
(60) Provisional application No. 63/071,236, filed on Aug. 27, 2020.

(51) Int. Cl. C12N 15/90; A61K 39/00; C07K 14/725; C07K 16/30; C12N 5/0783; C12N 9/22; C12N 15/11

(52) U.S. Cl. CPC C12N 15/907; A61K 39/4611; A61K 39/4632; A61K 39/464488; C07K 14/7051; C07K 16/3069; C12N 5/0636; C12N 9/22; C12N 15/111; C12N 2310/20; C12N 2510/00

(57) ABSTRACT

Disclosed herein, in certain embodiments, are vector-free methods of manufacturing engineered immune cells. In some embodiments, also disclosed herein are compositions comprising engineered immune cells obtained from the methods and processes described herein. In additional embodiments, disclosed herein are methods of treating a disease and kits using engineered immune cells obtained from the methods and processes described herein.

### CROSS-REFERENCE TO RELATED APPLICATIONS

This application is the U.S. National Stage of PCT/US2021/047710, filed Aug. 26, 2021, which claims a priority benefit to U.S. Provisional Patent Application No. 63/071,236, filed Aug. 27, 2020; the entirety of these applications is herein expressly incorporated by reference in their entirety.

### SEQUENCE LISTING

The instant application contains a Sequence Listing which is hereby incorporated by reference in its entirety. Said Sequence Listing text file was created on Nov. 10, 2023, is 18 bytes and is named 125400_1479_Sequence_Listing.txt.

### BACKGROUND

Novel treatments using T-cells engineered to express chimeric antigen receptors (CARs) or exogenous T cell receptors (TCRs) have resulted in promising immunotherapies for some types of cancer, primarily hematologic malignancies.

Chimeric antigen receptor (CAR) or T cell receptor (TCR) T cells are effector immune cells that are genetically modified to recognize a specific tumor-associated antigen and subsequently kill the tumor cell. Manufacturing of these cells generally includes stimulation of T cells, followed by transduction of the cells using a viral vector, such as a lentiviral vector, to introduce nucleic acids which encode the CAR or TCR for expression. One limitation of current vector transduction, such as lentiviral vector transduction, is their inability to efficiently mediate gene transfer into quiescent cells, such as primary T cells. Previous studies have shown that stimulation and expansion of T cells results in a more differentiated T cell phenotype. Undifferentiated or unstimulated T cell populations result in increased persistence and higher efficacy but low transfection efficiency. Further, manufacturing of T cells for adoptive therapies using lentiviral vectors requires up to 10-12 days before the cells can be harvested and formulated for administration.

Accordingly, a need exists for improved methods of T cell transduction with nucleic acids which encode an exogenous immune receptor while retaining an undifferentiated phenotype and decreasing manufacturing time. The present invention provides methods that address these needs.

### SUMMARY

In certain embodiments, disclosed herein are methods and processes for vector-free manufacturing of engineered immune cells. In some embodiments, also disclosed herein are compositions comprising engineered immune cells obtained from the methods and processes described herein. In additional embodiments, disclosed herein are methods of treating a disease and kits using engineered immune cells obtained from the methods and processes described herein.

In some embodiments, disclosed herein is a vector free method of preparing a population of modified unstimulated immune cells, comprising: (a) delivering into a population of unstimulated immune cells obtained from a biological sample (i) a gene-editing nuclease, (ii) a guide RNA, and (iii) a homology-directed repair (HDR) template comprising a polynucleotide encoding an antigen-binding polypeptide, by a transfection method; and (b) culturing the population under non-expansion conditions for about 72 hours or less, wherein the gene-editing nuclease and the guide RNA form a complex to generate a double-stranded break at a target site within at least one unstimulated immune cell, and wherein the HDR template facilitates HDR at the target site to generate at least one modified unstimulated immune cell within the population.

In some embodiments, disclosed herein is a vector free method of preparing a population of modified unstimulated immune cells, comprising: (a) obtaining an enriched population of unstimulated immune cells from a biological sample; (b) delivering into the enriched population (i) a gene-editing nuclease, (ii) a guide RNA, and (iii) a homology-directed repair (HDR) template comprising a polynucleotide encoding an antigen-binding polypeptide by a transfection method; and (c) culturing the population under non-expansion conditions; wherein the gene-editing nuclease and the guide RNA form a complex to generate a double-stranded break at a target site within a plurality of unstimulated immune cells within the population, and wherein the HDR template facilitates HDR at the target site to generate a plurality of modified unstimulated immune cells, and wherein about 10% or higher of the unstimulated immune cells within the population are modified.

In some embodiments, disclosed herein is a vector-free method of generating a population of modified unstimulated immune cells, comprising: (a) inducing a homology-direct repair (HDR) in about 10% or higher of a population of unstimulated immune cells by: (i) contacting the population of unstimulated immune cells with a gene-editing nuclease and a homology-directed repair (HDR) template comprising a polynucleotide encoding an antigen-binding polypeptide; and (ii) delivering into the unstimulated immune cells the gene-editing nuclease and the HDR template by a transfection method; and (b) culturing the unstimulated immune cells under non-expansion conditions for about 72 hours or less, thereby generating the population of modified unstimulated immune cells. In some embodiments, this method can further comprise (a) contacting the population of unstimulated immune cells with a guide RNA and delivering the guide RNA into the unstimulated immune cells by a transfection method, and optionally (b) wherein the gene-editing nuclease and optionally the guide RNA generate a double stranded break at a target site within the genome of one or more unstimulated immune cells. In yet other embodiments, in these methods the HDR template facilitates HDR at the target site in about 10% or higher of the population of unstimulated immune cells. Further, the population of unstimulated immune cells can be obtained from a biological sample; and/or can comprise an enriched population of CD4⁺ T cells, CD8⁺ T cells, or a combination thereof. In some aspects, the immune cells comprise T cells, natural killer cells, natural killer T cells, macrophages, monocytes, B cells, hematopoietic stem cells, or combinations thereof. In some aspects, the immune cells consist of T cells, natural killer cells, natural killer T cells, macrophages, monocytes, B cells, hematopoietic stem cells, or combinations thereof.

In some embodiments for all of the methods described herein, the population of unstimulated immune cells can comprise, for example, about 5 million cells, 10 million cells, 20 million cells, 50 million cells, 100 million cells, 500 million cells, 1 billion cells, 2 billion cells, 3 billion cells, 4 billion cells, 5 billion cells or more.

In some embodiments for all of the methods described herein, the transfection method can (a) provide an efficiency of from about 10% to about 99%, from about 12% to about 99%, from about 13% to about 99%, from about 15% to about 99%, from about 20% to about 99%, from 30% to about 99%, from about 40% to about 99%, from about 50% to about 99%, from about 60% to about 99%, from about 70% to about 99%, from about 10% to about 80%, from about 12% to about 80%, from about 13% to about 80%, from about 15% to about 80%, from about 20% to about 80%, from about 30% to about 80%, from about 40% to about 80%, from about 50% to about 80%, from about 20% to about 70%, or from about 30% to about 60%; and/or (b) provide an efficiency of about 10%, 12%, 13%, 15%, 18%, 20%, 25%, 30%, 35%, 40%, 45%, 50%, 60%, 70%, 80%, 90%, 95%, or 99%.

In some embodiments for all of the methods described herein, the method can provide a cell viability: (a) of from about 10% to about 99%, from about 12% to about 99%, from about 13% to about 99%, from about 15% to about 99%, from about 20% to about 99%, from 30% to about 99%, from about 40% to about 99%, from about 50% to about 99%, from about 60% to about 99%, from about 70% to about 99%, from about 10% to about 80%, from about 12% to about 80%, from about 13% to about 80%, from about 15% to about 80%, from about 20% to about 80%, from about 30% to about 80%, from about 40% to about 80%, from about 50% to about 80%, from about 20% to about 70%, or from about 30% to about 60%; and/or (b) of about 10%, about 12%, about 13%, about 15%, about 18%, about 20%, about 25%, about 30%, about 35%, about 40%, about 45%, about 50%, about 60%, about 70%, about 80%, about 90%, about 95%, or about 99%.

In some embodiments for all of the methods described herein, (a) about 1 pM to about 10 mM of the HDR template is delivered into the unstimulated immune cells; and/or (b) about 1 pM to about 10 mM of the gene-editing nuclease is delivered into the unstimulated immune cells; and/or (c) about 1 pM to about 10 mM of the guide RNA is delivered into the unstimulated immune cells.

In some embodiments for all of the methods described herein, (a) the ratio of the gene-editing nuclease to guide RNA is about 10:1, about 5:1, about 2:1, about 1:1, about 1:2, about 1:5, or about 1:10; and/or (b) the ratio of the HDR template to the complex formed between the gene-editing nuclease and the guide RNA is about 5:1, about 2:1, about 1:1, about 1:2, or about 1:5; and/or (c) the ratio of the HDR template to the gene-editing nuclease is about 5:1, about 2:1, 1:1, about 1:2, or about 1:5.

In some embodiments for all of the methods described herein, the complex is a ribonucleoprotein (RNP) complex.

In some embodiments for all of the methods described herein, the population of unstimulated immune cells: (a) comprises unstimulated T cells, unstimulated Natural Killer (NK) cells, unstimulated natural killer T (NKT) cells, or a combination hereof; and/or (b) comprises unstimulated T cells comprising CD4⁺ T cells, CD8⁺ T cells, CD4⁺/CD8⁺ T cells, or a combination thereof, and/or (c) comprises an enriched population of CD4⁺ T cells, CD8⁺ T cells, or a combination thereof.

In some embodiments for all of the methods described herein, the enriched population comprises about 90%, about 95%, about 99%, or about 100% CD4⁺ T cells, CD8⁺ T cells, or a combination thereof.

In some embodiments for all of the methods described herein, the method further comprises a step of incubating the biological sample with a plurality of CD4 and/or CD8 labeled microbeads, optionally magnetized microbeads, prior to generating the population of unstimulated immune cells. In another aspect, the step further comprises: (a) incubating the biological sample with a solution comprising albumin, optionally human serum albumin (HSA); and/or (b) a cell selection process to enrich the unstimulated cells in the population; and/or (c) incubating the enriched unstimulated cells in a cell media comprising minimum media, HSA, cytokines, supplements, or a combination thereof.

In some embodiments for all of the methods described herein, the modified unstimulated immune cells: (a) are cultured under non-expansion conditions for about 48 hours or less, about 36 hours or less, about 24 hours or less, or about 18 hours or less; and/or (b) are cultured under non-expansion conditions for about 18 hours, about 24 hours, about 36 hours, about 48 hours, or about 72 hours; and/or (c) are further resuspended in a cryopreservant solution and cryo-frozen.

In some embodiments for all of the methods described herein, the transfection method comprises electroporation or a cell squeezing method.

In some embodiments for all of the methods described herein, the antigen-binding polypeptide: (a) comprises an antigen-binding domain; (b) comprises a chimeric antigen receptor (CAR); (c) comprises a cell surface receptor ligand; or (d) comprises a T-cell receptor (TCR); and/or (e) binds to a tumor antigen. In another aspect, (a) the antigen-binding domain comprises a full length antibody or an antigen-binding fragment thereof, a Fab, a F(ab)₂, a monospecific Fab₂, a bispecific Fab₂, a trispecific Fab₂, a single-chain variable fragment (scFv), a diabody, a triabody, a minibody, a V-NAR, or a VhH; and/or (b) the CAR comprises the antigen-binding domain, a transmembrane domain, and an intracellular domain, and optionally wherein the CAR comprising a hinge region. In a further aspect, (a) the transmembrane domain is selected from an artificial hydrophobic sequence, a transmembrane domain of a type I transmembrane protein, an alpha, beta, or zeta chain of a T cell receptor, CD28, CD3 epsilon, CD45, CD4, CD5, CD8, CD9, CD16, CD22, CD33, CD37, CD64, CD80, CD86, OX40 (CD134), 4-1BB (CD137), ICOS (CD278), CD154, and a transmembrane domain derived from a killer immunoglobulin-like receptor (KIR); and/or (b) the intracellular domain comprises a costimulatory signaling domain and an intracellular signaling domain; and/or (c) the intracellular domain comprises one or more of a costimulatory domain of a protein selected from the group consisting of proteins in the TNFR superfamily, CD27, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS (CD278), NKG2C, B7-H3 (CD276), and an intracellular domain derived from a killer immunoglobulin-like receptor (KIR), or a variant thereof. In yet a further aspect, (a) the costimulatory domain comprises a 4-1BB (CD137) costimulatory domain; and/or (b) the intracellular signaling domain comprises an intracellular domain selected from the group consisting of cytoplasmic signaling domains of a human CD3 zeta chain (CD3ζ), FcγRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof, and/or (c) the intracellular signaling domain comprises a 4-1BB costimulatory domain and a human CD3 zeta chain (CD3ζ) cytoplasmic signaling domain. In yet another aspect, the cytoplasmic signaling domain comprises a human CD3 zeta chain (CD3ζ).

In methods where the antigen-binding polypeptide binds to a tumor antigen, the tumor antigen can be (a) associated with a hematologic malignancy; and/or (b) selected from CD19, CD20, CD22, and CD33/IL3Ra.; and/or (c) associated with a solid tumor; and/or (d) selected from ROR1, mesothelin, c-Met, PSMA, PSCA, Folate receptor alpha, Folate receptor beta, EGFRvIII, GPC2, TnMUC1, GDNF family receptor alpha-4 (GFRa4), fibroblast activation protein (FAP), and IL13Ra2.

In methods where the antigen-binding polypeptide comprises a T-cell receptor (TCR), the TCR can comprise a TCR alpha chain and a TCR beta chain; and/or is selected from a wild-type TCR, a high affinity TCR, and a chimeric TCR.

In some embodiments for all of the methods described herein, the HDR template can (a) further comprise a 5′ homology arm upstream of the polynucleotide; and/or (b) further comprise a 3′ homology arm downstream of the polynucleotide; and/or (c) be a double-stranded DNA template; and/or (d) can be a double-stranded DNA template, wherein the HDR template is from about 2 kilo-base pairs (kb) to about 5 kb, from about 2.3 kb to about 5 kb, from about 3 kb to about 5 kb, from about 3 kb to about 4 kb, from about 2 kb to about 4 kb, from about 2.3 kb to about 4 kb, from about 2 kb to about 3 kb, from about 2.3 kb to about 3 kb, or from about 4 kb to about 5 kb in length; and/or (e) be delivered by electroporation. In another aspect, the 5′ homology arm can be (a) adjacent to the polynucleotide; and/or (b) homologous to a genomic region 5′ of the target site; and/or (c) from about 50 nucleotides to about 500 nucleotides, from about 50 nucleotides to about 400 nucleotides, from about 50 to about 300 nucleotides, from about 50 nucleotides to about 200 nucleotides, from about 50 nucleotides to about 150 nucleotides, from about 100 nucleotides to about 500 nucleotides, from about 100 nucleotides to about 400 nucleotides, from about 100 nucleotides to about 300 nucleotides, from about 100 nucleotides to about 200 nucleotides, from about 200 nucleotides to about 500 nucleotides, from about 200 nucleotides to about 400 nucleotides, from about 200 nucleotides to about 300 nucleotides, from about 300 nucleotides to about 500 nucleotides, or from about 300 nucleotides to about 400 nucleotides in length; and/or (d) about 50, about 100, about 150, about 200, about 250, about 300, about 350, about 400, about 450, or about 500 nucleotides in length. In yet another aspect, the 3′ homology arm can be (a) adjacent to the polynucleotide; and/or (b) homologous to a genomic region 3′ of the target site; and/or (c) from about 50 nucleotides to about 500 nucleotides, from about 50 nucleotides to about 400 nucleotides, from about 50 to about 300 nucleotides, from about 50 nucleotides to about 200 nucleotides, from about 50 nucleotides to about 150 nucleotides, from about 100 nucleotides to about 500 nucleotides, from about 100 nucleotides to about 400 nucleotides, from about 100 nucleotides to about 300 nucleotides, from about 100 nucleotides to about 200 nucleotides, from about 200 nucleotides to about 500 nucleotides, from about 200 nucleotides to about 400 nucleotides, from about 200 nucleotides to about 300 nucleotides, from about 300 nucleotides to about 500 nucleotides, or from about 300 nucleotides to about 400 nucleotides in length; and/or (d) about 50, about 100, about 150, about 200, about 250, about 300, about 350, about 400, about 450, or about 500 nucleotides in length.

In some embodiments for all of the methods described herein, the target site is in the TRAC locus, and optionally exon 1 of the TRAC locus.

In some embodiments for all of the methods described herein, the gene-editing nuclease comprises: (a) a Cas nuclease; and/or (b) a zinc finger nuclease; and/or (c) a transcription activator-like effector nuclease (TALEN). In another aspect, the Cas nuclease: (a) is Cas9, optionally SpCas9 or SaCas9; and/or (b) and the guide RNA assembles into a complex prior to delivering into the unstimulated immune cells; and/or (c) the guide RNA assembles into a complex after delivering into the unstimulated immune cells.

In some embodiments for all of the methods described herein, the method further comprises delivering one or more additional guide RNA into the unstimulated immune cells.

In some embodiments for all of the methods described herein, the biological sample: (a) is a blood sample; and/or (b) is a blood sample, wherein the blood sample is a whole blood sample, a peripheral blood mononuclear cell (PBMC) sample, or an apheresis sample; and/or (c) is a blood sample, wherein the blood sample is an apheresis sample which is cryopreserved; and/or (d) is a blood sample, wherein the blood sample is an apheresis sample which is fresh.

In some embodiments for all of the methods described herein, the method further comprises stimulating the modified unstimulated immune cells to generate a population of modified stimulated immune cells, and optionally expanding the population of modified stimulated immune cells. In another aspect, the population of modified stimulated immune cells can be cultured under expansion conditions for about 1 day, about 2 days, about 3 days, about 4 days, about 5 days, about 6 days, about 7 days, about 8 days, about 9 days, about 10 days, about 11 days, about 12 days, or more.

In some embodiments, disclosed herein is a population of modified unstimulated immune cells generated by a method described herein.

In some embodiments, disclosed herein is a population of modified stimulated immune cells generated by a method described herein.

In some embodiments, disclosed herein is a composition comprising a population of modified unstimulated immune cells generated by a method described herein, or a population of modified stimulated immune cells generated by a method described herein; optionally comprising a pharmaceutically acceptable excipient.

In some embodiments, disclosed herein is a method of treating a disease in a subject in need thereof, comprising administering a population of modified unstimulated immune cells generated by a method described herein to the subject; or administering a population of modified stimulated immune cells generated by a method described herein to the subject, or administering the composition comprising the population of modified unstimulated immune cells or the population of modified stimulated immune cells.

In some embodiments, the subject has a cancer, such as a solid tumor. In other aspects of the methods of treating a disease described herein, the cancer is a hematologic malignancy. In yet other aspects of the methods of treating a disease described herein, the antigen-binding domain is specific for an antigen expressed by the cancer. In another aspect, the biological sample is autologous to the subject. Alternatively, the biological sample can be allogeneic to the subject. Finally, in all of the methods of treating disease described herein, the subject can be a human.

In some embodiments, disclosed herein is a kit comprising a population of modified unstimulated immune cells obtained from a method described herein or a population of modified stimulated immune cells obtained from a method described herein.

In some embodiments, disclosed herein is a vector free method of preparing a population of modified unstimulated cells selected from cells of the liver, skin, or pancreas, comprising: (a) delivering into a population of unstimulated cells obtained from a biological sample (i) a gene-editing nuclease, (ii) a guide RNA, and (iii) a homology-directed repair (HDR) template comprising a polynucleotide encoding an antigen-binding polypeptide, by a transfection method; and (b) culturing the population under non-expansion conditions for about 72 hours or less, wherein the gene-editing nuclease and the guide RNA form a complex to generate a double-stranded break at a target site within at least one unstimulated cell, and wherein the HDR template facilitates HDR at the target site to generate at least one modified unstimulated cell within the population.

In some embodiments, disclosed herein is a vector free method of preparing a population of modified unstimulated cells selected from cells of the liver, skin, or pancreas, comprising: (a) obtaining an enriched population of unstimulated cells from a biological sample; (b) delivering into the enriched population (i) a gene-editing nuclease, (ii) a guide RNA, and (iii) a homology-directed repair (HDR) template comprising a polynucleotide encoding an antigen-binding polypeptide by a transfection method; and (c) culturing the population under non-expansion conditions; wherein the gene-editing nuclease and the guide RNA form a complex to generate a double-stranded break at a target site within a plurality of unstimulated cells within the population, and wherein the HDR template facilitates HDR at the target site to generate a plurality of modified unstimulated cells, and wherein about 10% or higher of the unstimulated cells within the population are modified.

In some embodiments, the liver cells may be selected from one or more of hepatocytes, hepatic stellate cells, sinusoidal endothelial cells, and Kupffer cells. In some embodiments, the skin cells may be selected from one or more of keratinocytes, melanosomes, melanocytes, Langerhans cells, and Merkel cells. In some embodiments, the pancreatic cells may be selected from one or more of pancreatic alpha cells, pancreatic beta cells, pancreatic delta cells, pancreatic gamma cells, and pancreatic epsilon cells.

Both the foregoing summary and the following description of the drawings and detailed description are exemplary and explanatory. They are intended to provide further details of the disclosure, but are not to be construed as limiting. Other objects, advantages, and novel features will be readily apparent to those skilled in the art from the following detailed description of the disclosure

### BRIEF DESCRIPTION OF THE DRAWINGS

FIGS. 1A-1C illustrate exemplary schematics of vector-free manufacturing processes described herein. FIG. 1A illustrates a schematic of a vector-free manufacturing process of an engineered immune cell. FIG. 1B illustrates a schematic of the manufacturing process of clinical-grade vector-free engineered T cells. FIG. 1C illustrates a schematic of allogeneic T-cell manufacturing process.

FIGS. 2A and 2B illustrate an exemplary schematic for a vector-free manufacturing process of an engineered T cell. FIG. 2A details the manufacturing process at Day 0 and FIG. 2B details the manufacturing process at Day 0 to Day 3/Harvest.

FIGS. 3A and 3B illustrate disruption of endogenous TCR expression in unstimulated T cells via CRISPR/Cas9 using TRAC-targeting gRNA. CRISPR/Cas9 gene editing was performed to target the TCR alpha constant (TRAC) locus in unstimulated T cells. Two CRISPR/Cas9 ribonucleoprotein (RNP) systems were tested: Truecut.v2 Cas9 (ThermoFisher Scientific) and SpyFi Cas9 (Aldevron). FIG. 3A depicts % CD3+ TCR+/Live Cells for positive control, Truecut and SpyFi. FIG. 3B also depicts % CD3+ TCR+/Live Cells for positive control, Truecut and SpyFi.

FIG. 4 illustrates an exemplary schematic for introducing a donor DNA containing an EcoRI site into exon 1 of the TRAC locus via homology-directed repair of double-stranded DNA breaks.

FIGS. 5A and B illustrate a gel electrophoresis images of PCR amplicons digested with EcoRI to demonstrate HDR-mediated insertion into chemically-modified ssDNA donors or “ultramers”.

FIG. 6 is an exemplary schematic illustrating a knock-in strategy to insert a donor DNA encoding the NY-ESO-1 TCR into the TRAC locus.

FIG. 7 illustrates the targeted insertion of a GFP HDR cassette into a T-cell genome.

### DETAILED DESCRIPTION

### I. Overview

CAR-T and TCR cells offer exciting promise to cancer patients. However, several challenges currently exist relating to manufacturing of CAR-T and TCR cells, and these challenges impact the potential success of these cancer therapeutics. First, at present current CAR-T and TCR manufacturing processes generally utilize viral vector systems for propagation. However, using this type of propagation method results in an about 9 day time period prior to cell harvesting. The present disclosure details surprising vector-free methods which dramatically reduce the time to CAR-T and/or TCR cell harvesting, e.g., to about 72 hours or less.

A second challenge existing with present technology and which is addressed by the present disclosure is the need to scale up production. The present disclosure details a vector-free process which is surprisingly scaleable with an acceptable insertion efficiency, even with large scale production. In particular, the short process time impacts the ability to scale up the process. Thus, it was surprising that a short CAR-T and TCR manufacturing process could be designed, which can be done on a large scale production level, and which has a high percentage of insertion efficiency.

Another surprising aspect of the present disclosure is the impact of culturing stimulated vs unstimulated cells. In general, insertion efficiency is higher with stimulated cells, but manufacturing processes are easier with unstimulated cells. One issue is that moving cells between medium results in high cell loss. In addition, the use of undifferentiated cells can produce a better product as the use of undifferentiated cells can result in a plurality of different unstimulated immune cell populations, e.g., CD4+ or CD8+ T cells (e.g., cells having different and not uniform properties). The methods described herein can be used with stimulated cells (producing higher insertion efficiency) or unstimulated cells (can produce an end product having less differentiated and more potent phenotypes).

These advantages and discoveries are described in more detail below.

### II. Methods of Manufacturing Engineered Immune Cells

In certain embodiments, disclosed herein are methods of preparing a modified immune cell or precursor cell thereof (e.g., a modified T cell, a modified natural killer (NK) cell, a modified natural killer T (NKT) cell, a modified macrophage, a modified monocyte, a modified B cell, or a modified hematopoietic stem cell). In some embodiments, the modified immune cell or precursor cell thereof is a modified unstimulated immune cell or precursor cell thereof (e.g., a modified unstimulated T cell, a modified unstimulated NK cell, a modified unstimulated NKT cell, a modified unstimulated macrophage, a modified unstimulated monocyte, a modified unstimulated B cell, or a modified unstimulated hematopoietic stem cell) which expresses an exogenous antigen-binding polypeptide. In some embodiments, the antigen-binding polypeptide comprises a chimeric antigen receptor (CAR) and/or an exogenous T cell receptor (TCR). In some embodiments, the antigen-binding polypeptide comprises an antigen-binding domain, a cell surface receptor ligand, or a polypeptide that binds to a tumor antigen. In some embodiments, the method is a Good Manufacturing Practice (GMP) method, e.g., in compliance with U.S. Food and Drug Administration (FDA) regulations or in compliance with an equivalent of the U.S. FDA in a foreign jurisdiction.

In some embodiments, the methods of manufacturing engineered cells are not limited to immune cells, but further include mammalian cells from the liver, skin, and pancreas. Any and all methods recited as applicable to the immune cells described herein are further applicable to cells from the liver, skin, and pancreas. In some embodiments, the cells from the liver, skin, and pancreas are not subjected to stimulation. In some embodiments, liver cells may be selected from one or more of hepatocytes, hepatic stellate cells, sinusoidal endothelial cells, and Kupffer cells. In some embodiments, skin cells may be selected from one or more of keratinocytes, melanosomes, melanocytes, Langerhans cells, and Merkel cells. In some embodiments, pancreatic cells may be selected from one or more of pancreatic alpha cells, pancreatic beta cells, pancreatic delta cells, pancreatic gamma cells, and pancreatic epsilon cells.

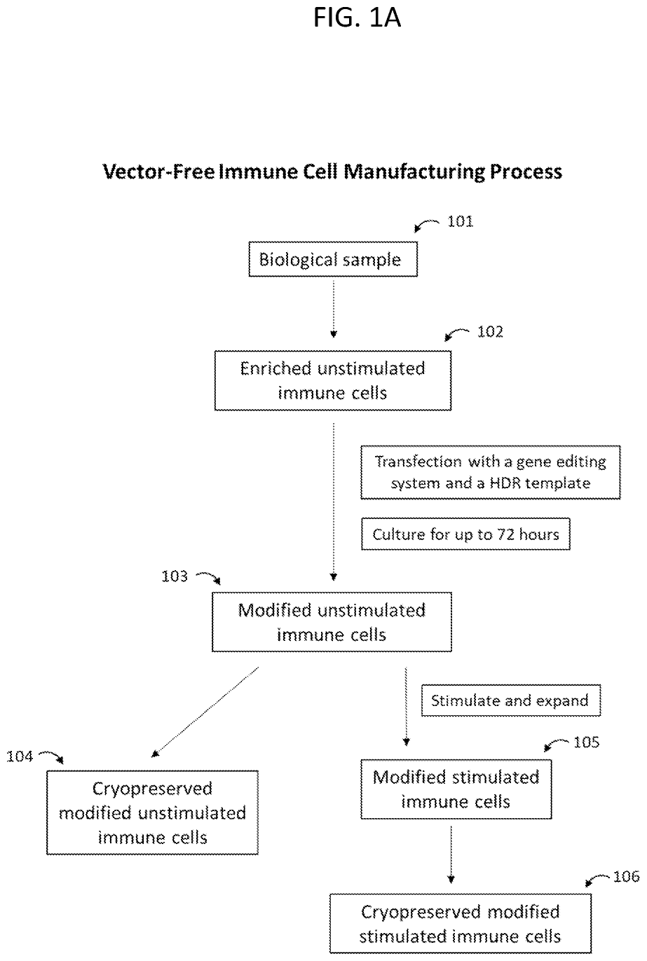

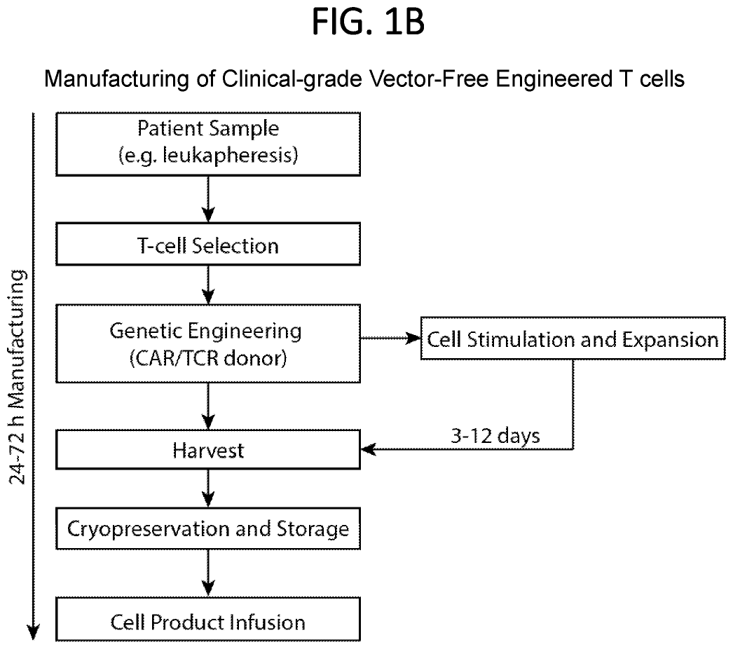

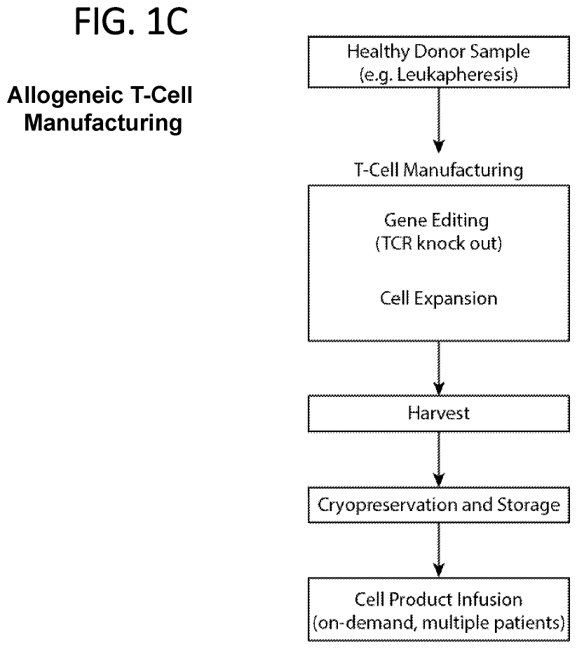

In some embodiments, a manufacturing method of preparing an engineered or modified immune cell or precursor cell thereof is illustrated in FIG. 1A. As shown in FIG. 1, a biological sample 101 is processed through a cell separation system to generate a population of enriched unstimulated immune cells 102 (e.g., enriched unstimulated CD4+ and CD8+ T cells). Next, the enriched unstimulated immune cells 102 (e.g., enriched unstimulated CD4+ and CD8+ T cells) are transfected with a gene editing system and a template (e.g., a HDR template) and subsequently cultured for up to about 72 hours to generate a population of modified unstimulated immune cells 103 (e.g., modified unstimulated CD4+ and CD8+ T cells). The modified unstimulated immune cells 103 are then cryofrozen to generate cryopreserved modified unstimulated immune cells 104. Optionally, the modified unstimulated immune cells 104 can be stimulated and expanded for up to about 10 to about 12 days to generate modified stimulated immune cells 105. The modified stimulated immune cells 105 can further be cryoprotected to generate cryopreserved modified stimulated immune cells 106. In some instances, the manufacturing method is a vector-free manufacturing method of preparing a modified immune cell or precursor cell thereof. Also see FIG. 1B and FIG. 1C, which respectively illustrate a schematic of the manufacturing process of clinical-grade vector-free engineered T cells and allogeneic T-cells. In some cases, the manufacturing method improves efficiency of transfection, yields of the modified unstimulated or stimulated immune cells, improves cell viability, or any combination thereof.

A. Selection and Enrichment of Unstimulated Immune Cells

In some embodiments, an unstimulated immune cell described herein is an unstimulated T cell. The T cell can be a cytotoxic T cell, a regulatory T cell, or a NKT cell. In exemplary embodiments, the T cell is a CD8+ T cell or a CD4+ T cell. In some embodiments, a population of unstimulated immune cells are harvested from a biological sample from a subject, e.g., a tissue, fluid, or other sample from the subject. In some embodiments, the biological sample is a tissue or organ sample, e.g., liver, lung, stomach, intestine, colon, kidney, pancreas, breast, bone, prostate, cervix, testes, ovaries, tonsil, spleen, lymph node, or tumor tissue, or cells derived therefrom. In some embodiments, the biological sample is a fluid sample, e.g., a blood, plasma, serum, cerebrospinal fluid, synovial fluid, urine, or sweat sample. In some embodiments, the biological sample is a blood sample, optionally selected from a whole blood sample, a peripheral blood mononuclear cell (PBMC) sample, or an apheresis sample. In some embodiments, the sample is from a xenogeneic source, for example, from mouse, rat, non-human primate, or pig.

In some embodiments, the biological sample is an apheresis sample from a patient. In some embodiments, the apheresis sample is a leukapheresis sample. In some embodiments, the apheresis sample (e.g., the leukapheresis sample) is cryopreserved prior to harvesting of the immune cell or population of immune cells. In some embodiments, the apheresis sample (e.g., the leukapheresis sample) is a fresh apheresis sample from a patient that has not been cryopreserved. In some embodiments, the immune cell or population of immune cells are obtained from an apheresis sample (e.g., an leukapheresis sample) during a process or protocol which comprises an enrichment step.

In some embodiments, a population of unstimulated immune cells, in particular unstimulated T cells, are isolated and enriched in one or more selection steps, e.g., more than one depletion step (e.g., removal of non-immune or non-T cells). In some instances, the isolation step further includes one or more separation steps, including separation based on one or more properties, such as size, density, sensitivity or resistance to particular reagents, and/or affinity, e.g., immunoaffinity, to antibodies or other binding partners. In some aspects, the isolation is carried out using the same apparatus or equipment sequentially in a single process stream and/or simultaneously. In some aspects, the isolation, culture, and/or engineering of the different populations is carried out from the same starting composition or material, such as from the same sample.

In some aspects, a population of unstimulated immune cells is isolated in a closed system or apparatus, and/or in the same vessel or set of vessels, e.g., same (or same set of) unit, chamber, column, e.g., magnetic separation column, tube, tubing set, culture or cultivation chamber, culture vessel, processing unit, cell separation vessel, centrifugation chamber. For example, in some cases, isolation of the population of unstimulated immune cells is carried out on a system or apparatus employing a single or the same isolation or separation vessel or set of vessels, such as a single column or set of columns, and/or same tube, or tubing set, for example, without requirements to transfer the cell population, composition, or suspension from one vessel, e.g., tubing set, to another.

In some instances, by employing simultaneous or sequential selections, a plurality of different unstimulated immune cell populations, e.g., CD4+ or CD8+ T cells, are selected, enriched, and/or isolated. In some embodiments, the isolation step includes separation of different cell types based on the expression or presence of one or more specific molecules in the cell, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the unstimulated immune cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells. For example, in some aspects, a selection of the CD4+ and CD8+ populations enrich the populations, but can also comprise some residual or small percentage of other non-selected cells. In such instances, the residual percentage of other non-selected cells can be less than about or equal to about 10%, less than about or equal to about 9%, less than about or equal to about 8%, less than about or equal to about 7%, less than about or equal to about 6%, less than about or equal to about 5%, less than about or equal to about 4%, less than about or equal to about 3%, less than about or equal to about 2%, less than about or equal to about 1%, less than about or equal to about 0.5%, or less than about or equal to about 0.1%, or less.

In some examples, multiple rounds of separation steps can be carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

In some embodiments, selection and enrichment of an unstimulated T cell population by positive or negative selection can be accomplished, e.g., with a combination of antibodies directed to surface markers unique to the positively or negatively selected cells. In some embodiments, a cocktail of monoclonal antibodies directed to cell surface markers present on CD4+ T cells include antibodies against CD45RA, CCR7, CD62L, CD127 (IL-7Rα), and/or CD132. In some embodiments, a cocktail of monoclonal antibodies directed to cell surface markers present on CD8+ T cells include antibodies against CD62L, CCR7, and/or CD127 (IL-7Ra). In additional embodiments, a cocktail of monoclonal antibodies directed to cell surface markers present on CD4+ and/or CD8+ T cells include antibodies against CD2, CD3, CD27, and/or TCR. In some embodiments, a cocktail of monoclonal antibodies directed to cell surface markers present on CD4− and CD8− cells by negative selection include antibodies against CD14, CD20, CD11b, CD16, and/or HLA-DR.

In some embodiments, the method comprises separate selection of CD4+ and CD8+ T cells. In some aspects, the methods include a first positive selection for CD4+ T cells in which the non-selected cells (CD4− cells) from the first selection are used as the source of cells for a second positive selection to enrich for CD8+ T cells. In other aspects, the methods include a first positive selection for CD8+ T cells in which the non-selected cells (CD8− cells) from the first selection are used as the source of cells for a second position selection to enrich for CD4+ T cells.

In some embodiments, the method comprises a simultaneous selection of CD4+ T cells and CD8+ T cells. In some aspects, the method comprises a positive selection, in which the CD4+ and CD8+ T cells are selected and the CD4− and CD8− cells are subsequently removed.

In some embodiments, the methods of isolating, selecting, and/or enriching for unstimulated immune cells, such as by positive or negative selection based on the expression of a cell surface marker or markers, for example by any of the methods described above, can include immunoaffinity-based selections. In some embodiments, the immunoaffinity-based selections include contacting a sample containing cells, such as primary human T cells comprising CD4+ and CD8+ cells, with an antibody or binding partner that specifically binds to the cell surface marker or markers. In some embodiments, the antibody or binding partner is bound to a solid support or matrix, such as a sphere or bead, for example microbeads, nanobeads, including agarose, magnetic bead or paramagnetic beads, to allow for separation of cells for positive and/or negative selection. In some embodiments, the spheres or beads can be packed into a column to effect immunoaffinity chromatography, in which a sample containing cells, such as primary human T cells containing CD4+ and CD8+ cells, is contacted with the matrix of the column and subsequently eluted or released therefrom.

In some aspects, the sample or composition of unstimulated immune cells to be separated is incubated with small, magnetizable or magnetically responsive material, such as magnetically responsive particles or microparticles, such as paramagnetic beads. The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select. Such beads are known and are commercially available from a variety of sources including, in some aspects, DYNABEADS® (Life Technologies, Carlsbad, Calif.), MACS® beads (Miltenyi Biotec, San Diego, Calif) or STREPTAMER® bead reagents (IBA, Germany).

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In some embodiments, the affinity-based selection is via magnetic-activated cell sorting (MACS) (Miltenyi Biotech, Auburn, Calif.). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In some embodiments, the antibody specifically binding a cell surface marker associated with or coated on a bead or other surface is a full-length antibody or is an antigen-binding fragment thereof, including a (Fab) fragments, F(ab′)₂ fragments, Fab′ fragments, Fv fragments, variable heavy chain (VH) regions capable of specifically binding the antigen, single chain antibody fragments, including single chain variable fragments (scFv), and single domain antibodies (e.g., sdAb, sdFv, nanobody) fragments. In some embodiments, the antibody is a Fab fragment. In some embodiments, the antibody can be monovalent, bivalent or multivalent. In some embodiments, the antibody, such as a Fab, is a multimer. In some embodiments, the antibody, such as a Fab multimer, forms a multivalent complex with the cell surface marker.

For isolation of a desired population of unstimulated immune cells by positive or negative selection, the concentration of cells and surface (e.g., particles such as beads) can be varied. In certain aspects, it may be desirable to significantly decrease the volume in which beads and cells are mixed together (e.g., increase the concentration of cells), to ensure maximum contact of cells and beads. For example, in one aspect, a concentration of 10 billion cells/ml, 9 billion cells/ml, 8 billion cells/ml, 7 billion/ml, 6 billion/ml, or 5 billion/ml is used. In one aspect, a concentration of 1 billion cells/ml is used. In yet one aspect, a concentration of cells from 75, 80, 85, 90, 95, or 100 million cells/ml is used. In further aspects, concentrations of 125 or 150 million cells/ml can be used.

In some embodiments, separation and enrichment of unstimulated immune cells are carried out using an automated system, such as for example, using the CliniMACS system. In some aspects, the method uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In some embodiments, the separation and enrichment of unstimulated immune cells are carried out using automated system such as a CliniMACS Plus (Miltenyi) system in combination with a filtration system such as the LOVO Cell Processing System (Fresenius Kabi). In some instances, unstimulated immune cells are separated from the supernatant in a spinning membrane filtration step. In particular, cells of a certain cut-off range, e.g., <4 μm, along with the supernatant pass through the membrane pores and are removed while cells greater than 4 μm are retained within a filter chamber and subsequently harvested. The harvested cells are further processed through the CliniMACS Plus system.

In some embodiments, separation and enrichment steps are carried out using a system equipped with a cell processing unit that permits automated washing and fractionation of cells by centrifugation. In some aspects, the separation and enrichment steps are carried out using the CliniMACS Prodigy system (Miltenyi Biotec). A system with a cell processing unit can also include an onboard camera and image recognition software that determines the optimal cell fractionation endpoint by discerning the macroscopic layers of the source cell product. For example, peripheral blood is automatically separated into erythrocytes, white blood cells and plasma layers. A cell processing system, such as the CliniMACS Prodigy system, can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al. (2012) J Immunother. 35(9): 651-660, Terakura et al. (2012) Blood. 1:72-82, and Wang et al. (2012) J Immunother. 35(9):689-701.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al., Lab Chip, 10:1567-1573 (2010); and Godin et al., J. Biophoton., 1(5):355-376 (2008). In both cases, cells can be labeled with multiple markers, allowing for the isolation of well-defined T cell subsets at high purity.

In some embodiments, separation and enrichment of unstimulated immune cells are carried out using an elutriation system, which separates and purifies cells base on both size and density. In some instances, fluid passes through the unstimulated immune cell layer which is established within a centrifugal field inside a separation chamber. By varying the flow of the fluid in the opposite direction to the centrifugal field, the system aligns and collects particles according to size and density. In some instances, the elutriation system is ELUTRA from Terumo BCT or ROTEA from ThermoFisher.

In some embodiments, the unstimulated immune cells are incubated in a cell media during the one or more separation and enrichment steps. In some instances, the cell media is a complete cell media. In some instances, the cell media is a fetal bovine serum (FBS)-based media, e.g., comprising from about 1% to about 10% FBS. In some instances, the cell media is a chemically-defined media. In some instances, the cell media is a minimum media. Exemplary media for incubating the unstimulated immune cells during the separation and enrichment step include, but are not limited to, CliniMACS buffer from Miltenyi Biotech.

In some instances, the cell media further comprises a protein that coats the inner surface of the culture vessel without interacting with the cultured unstimulated immune cells. In some cases, the media comprises from about 0.1% to about 5% w/v or from about 0.5% to about 2% w/v of the protein. In some cases, the media comprises about 0.1%, about 0.2%, about 0.3%, about 0.4%, about 0.5%, about 0.6%, about 0.7%, about 0.8%, about 0.9%, or about 1% w/v of the protein. In some cases, the protein is an isolated recombinant protein or a fragment thereof. In some cases, the protein is an engineered de novo polypeptide. In some cases, the protein is a naturally-occurring protein or fragment thereof. In some cases, the protein is albumin (e.g., human serum albumin or HSA).

In some embodiments, the media comprises from about 0.1% to about 5% w/v or from about 0.5% to about 2% w/v of albumin (e.g., HSA). In some instances, the media comprises about 0.1%, about 0.2%, about 0.3%, about 0.4%, about 0.5%, about 0.6%, about 0.7%, about 0.8%, about 0.9%, or about 1% w/v of albumin (e.g., HSA). In some cases, the albumin (e.g., HSA) is a full-length albumin. In other cases, the albumin (e.g., HSA) is a fragment thereof, e.g., without the signaling peptide.

In some embodiments, the unstimulated immune cells collected from the one or more of the separation and enrichment steps described above are resuspended in a cell media. In some instances, the cell media is a complete cell media, optionally a complete serum-free and xeno-free media. In some instances, the media is a fetal bovine serum (FBS)-based media, e.g., comprising from about 1% to about 10% FBS. In some instances, the cell media is a chemically defined media. In some instances, the cell media is a minimum media. In some instances, the cell media further comprises a T-cell supplement, human serum, one or more cytokines such as IL-7 and/or IL-15, or a combination thereof. In some instances, the T-cell supplement is included in the media at a final concentration of about 2.4% v/v. In some instances, the human serum is included in the media at a final concentration of about 5%. In some instances, the media comprises L-glutamine or a L-glutamine substitute (e.g., L-alanine-L-glutamine), optionally at a final concentration of about 2 mM. In some instances, the media comprises IL-7 at a final concentration of about 5 ng/mL. In some instances, the media comprises IL-15 at a final concentration of about 5 ng/mL. In some embodiments, the cell media comprises CTS™ OPTMIZER™ from ThermoFisher, GlutaMAX™ Supplement from ThermoFisher, or a combination thereof.

In some embodiments, the separation and enrichment steps of the unstimulated immune cells are performed at a temperature of from about 2° C. to about 40° C. In some instances, the separation and enrichment steps are performed at a temperature of from about 2° C. to about 37° C., about 2° C. to about 35° C., about 2° C. to about 33° C., about 2° C. to about 30° C., about 2° C. to about 28° C., about 2° C. to about 26° C., about 2° C. to about 25° C., about 2° C. to about 20° C., about 2° C. to about 18° C., about 2° C. to about 15° C., about 2° C. to about 10° C., about 2° C. to about 8° C., about 4° C. to about 37° C., about 4° C. to about 35° C., about 4° C. to about 33° C., about 4° C. to about 30° C., about 4° C. to about 28° C., about 4° C. to about 26° C., about 4° C. to about 25° C., about 4° C. to about 20° C., about 4° C. to about 18° C., about 4° C. to about 15° C., about 4° C. to about 10° C., about 4° C. to about 8° C., about 20° C. to about 37° C., about 20° C. to about 35° C., about 20° C. to about 33° C., about 20° C. to about 30° C., about 20° C. to about 28° C., about 20° C. to about 26° C., about 20° C. to about 25° C., about 22° C. to about 25° C., about 22° C. to about 28° C., about 24° C. to about 30° C., about 24° C. to about 28° C., or about 25° C. to about 30° C. In some cases, the separation and enrichment steps are performed at a temperature of from about 2° C. to about 8° C. In some cases, the separation and enrichment are performed at a temperature of about 4° C.

In some embodiments, the unstimulated immune cell is an autologous immune cell. In other embodiments, the unstimulated immune cell is an allogeneic immune cell.

In some embodiments, the population of unstimulated immune cells comprises a plurality of T cells, optionally a plurality of CD4+ T cells, a plurality of CD8+ T cells, or a combination thereof. In some embodiments, a ratio of the CD8+ T cell to the CD4+ T cell in the population of immune cells is about 1:1, about 1:2, about 1:3, about 1:4, about 2:1, about 3:1, or about 4:1. In some instances, the ratio of the CD8+ T cell to the CD4+ T cell in the population of immune cells is about 1:1. In some instances, the ratio of the CD8+ T cell to the CD4+ T cell in the population of immune cells is about 1:2. In some instances, the ratio of the CD8+ T cell to the CD4+ T cell in the population of immune cells is about 2:1.

In some embodiments, the method described herein yields from about 50 million to about 50 billion enriched unstimulated immune cells, optionally from about 50 million to about 1 billion, from about 100 million to about 5 billion, from about 1 billion to about 10 billion, or from about 1 billion to about 50 billion cells. In some instances, the enriched unstimulated immune cells are obtained from a starting sample of about 50-500 mL of biological sample.

In some embodiments, the enriched population of unstimulated immune cells comprises about 75%, about 80%, about 90%, about 91%, about 92%, about 93%, about 94%, about 95%, about 96%, about 97%, about 98%, about 99%, or about 100% CD4⁺ T cells, CD8⁺ T cells, or a combination thereof.

B. Gene Edited Unstimulated Immune Cells

In certain embodiments, an unstimulated immune cell (e.g., unstimulated T cell) is modified by a gene editing system. In some embodiments, the unstimulated immune cell is genetically edited to disrupt the expression of one or more endogenously expressed genes. In some embodiments, the unstimulated immune cell has a reduction, deletion, elimination, knockout, or disruption in expression of an endogenous receptor (e.g., an endogenous T cell receptor). In some embodiments, the unstimulated immune cell has an exogenous immune receptor (e.g., a CAR or TCR) inserted into an endogenous gene locus. In some embodiments, the endogenous gene locus is TRAC.

In certain embodiments, an unstimulated immune cell is genetically edited to disrupt the expression of endogenous TCR gene products (e.g., gene products of TRAC and TRBC). Without being bound to any theory, disrupting the expression of TRAC and/or TRBC results in 1) reduced endogenous TCR and exogenous TCR mispairing, thus reducing the risk of autoreactivity; and 2) enhances exogenous TCR expression on the cell surface by reducing mispairing with endogenous TCR, thus increasing efficacy of the modified cells.

In some embodiments, an unstimulated immune cell comprises a genetic modification to disrupt the endogenous TRAC locus and insertion of an exogenous antigen-binding immune receptor. In some embodiments, the genetic modification to disrupt the endogenous TRAC locus is in an exon of TRAC. In some embodiments, the genetic modification is in an intron of TRAC. In some embodiments, the genetic modification to disrupt the endogenous TRAC locus is in exon 1 of the TRAC locus. In some embodiments, the exogenous antigen-binding immune receptor is a CAR. In some embodiments, the exogenous antigen-binding immune receptor is a TCR.

In some aspects, the gene editing system comprises an RNA-guided nuclease such as a clustered regularly interspersed short palindromic nucleic acid (CRISPR)-Cas system. The CRISPR system (also referred to herein as the CRISPR-Cas system, Cas system, or CRISPR/Cas system) comprises a Cas endonuclease and a guide nucleic acid sequence specific for a target gene which after introduction into a cell form a complex that enables the Cas endonuclease to introduce a break (e.g., a double stranded break) at the target gene.

In some embodiments, the Cas endonuclease comprises a Cas9 endonuclease. In some instances, the Cas9 endonuclease is derived from or based on, e.g., a Cas9 molecule of S. pyogenes (e.g., SpCas9), S. thermophiles, Staphylococcus aureus (e.g., SaCas9), or Neisseria meningitides. In some instances, the Cas9 endonuclease is derived from or based on, e.g., a Cas9 molecule of Acidovorax avenae, Actinobacillus pleuropneumoniae, Actinobacillus succinogenes, Actinobacillus suis, Actinomyces sp., cycliphilus denitrificans, Aminomonas paucivorans, Bacillus cereus, Bacillus smithii, Bacillus thuringiensis, Bacteroides sp., Blastopirellula marina, Bradyrhiz obium sp., Brevibacillus latemsporus, Campylobacter coli, Campylobacter jejuni, Campylobacter lad, Candidatus Puniceispirillum, Clostridiu cellulolyticum, Clostridium perfringens, Corynebacterium accolens, Corynebacterium diphtheria, Corynebacterium matruchotii, Dinoroseobacter sliibae, Eubacterium dolichum, gamma proteobacterium, Gluconacetobacler diazotrophicus, Haemophilus parainfluenzae, Haemophilus sputorum, Helicobacter canadensis, Helicobacter cinaedi, Helicobacter mustelae, Ilyobacler polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria baciliformis, Neisseria cinerea, Neisseria flavescens, Neisseria lactamica. Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tislrella mobilis, Treponema sp., or Verminephrobacter eiseniae.

In some embodiments, the Cas9 endonuclease is derived from a Cas9 molecule of: S. pyogenes (e.g., strain SF370, MGAS 10270, MGAS 10750, MGAS2096, MGAS315, MGAS5005, MGAS6180, MGAS9429, NZ131 and SSI-1), S. thermophilus (e.g., strain LMD-9), S. pseudoporcinus (e.g., strain SPIN 20026), S. mutans (e.g., strain UA 159, NN2025), S. macacae (e.g., strain NCTC1 1558), S. gallolylicus (e.g., strain UCN34, ATCC BAA-2069), S. equines (e.g., strain ATCC 9812, MGCS 124), S. dysdalactiae (e.g., strain GGS 124), S. bovis (e.g., strain ATCC 700338), S. cmginosus (e.g.; strain F0211), S. agalactia (e.g., strain NEM316, A909), Listeria monocytogenes (e.g., strain F6854), Listeria innocua (L. innocua, e.g., strain Clip 11262), Enterococcus italicus (e.g., strain DSM 15952), or Enterococcus faecium (e.g., strain 1,23,408).

In some instances, the endonuclease comprises Cas3, Cas8a, Cas8b, Cse1, Csy1, Csn2, Cas4, Cas10 (e.g., Cas10d), Cas12a (or Cpf1), Cas12b (or C2cl), Cas12d, Cas12e, Cas12f, Cas12g, Cas12h, Cas12i, Cas13, Cas14, Csm2, or Cmr5.

In some embodiments, the guide nucleic acid is a guide RNA (gRNA) molecule which directs the Cas-RNA complex to a target sequence. In some instances, the directing is accomplished through hybridization of a portion of the gRNA to DNA (e.g., through the gRNA targeting domain), and by binding of a portion of the gRNA molecule to the RNA-guided nuclease or other effector molecule (e.g., through at least the gRNA tracr). In some embodiments, a gRNA molecule consists of a single contiguous polynucleotide molecule, referred to herein as a “single guide RNA” or “sgRNA”. In other embodiments, a gRNA molecule consists of a plurality, usually two, polynucleotide molecules, which are themselves capable of association, usually through hybridization, referred to herein as a “dual guide RNA” or “dgRNA”.

In some cases, the gRNA molecule comprises a crRNA and a tracr, which can be optionally on a single polynucleotide or on separate polynucleotides. In some instances, the crRNA comprises a targeting domain and a region that interacts with a tracr to form a flagpole region. The tracr comprises the portion of the gRNA molecule that binds to a nuclease or other effector molecule. In some embodiments, the tracr comprises a nucleic acid sequence that binds specifically to a Cas endonuclease (e.g., Cas9). In some embodiments, the tracr comprises a nucleic acid sequence that forms part of the flagpole. In some embodiments, the targeting domain is the portion of the gRNA molecule that recognizes, e.g., is complementary to, a protospacer sequence within the target DNA.

A protospace-adjacent motif (PAM) is a 2-6 base pair DNA sequence located adjacent to the 3′ terminus of the protospacer and recognized by the Cas endonuclease. In some instances, each Cas endonuclease recognizes a specific PAM sequence. Exemplary PAM sequences include NGG sequence recognized by the S. pyogenes Cas9 endonuclease; or NGGNG or NNAGAAW sequence recognized by the S. thermophilus Cas9 endonuclease, where N is any nucleotide. One skilled in the art would understand how to design a gRNA molecule based on the specific Cas endonuclease used along with the PAM sequence in which the Cas endonuclease would recognize.

In some embodiments, one or more, two or more, three or more, or four or more guide nucleic acids (e.g., guide RNA molecules) are transfected into an immune cell with a Cas endonuclease. In some cases, about one, two, or three guide nucleic acids (e.g., guide RNA molecules) are transfected into an immune cell with a Cas endonuclease. In some cases, about three guide nucleic acids (e.g., guide RNA molecules) are transfected into an immune cell with a Cas endonuclease. In some cases about two guide nucleic acids (e.g., guide RNA molecules) are transfected into an immune cell with a Cas endonuclease. In some cases, about one guide nucleic acid (e.g., guide RNA molecule) is transfected into an immune cell with a Cas endonuclease.

In some embodiments, the gene editing system is a TALEN gene editing system. TALENs are produced artificially by fusing a TAL effector DNA binding domain to a DNA cleavage domain. Transcription activator-like effects (TALEs) can be engineered to bind to a target DNA. By combining an engineered TALE with a DNA cleavage domain, a restriction enzyme can be produced which is specific to any target DNA sequence.

TALEs are proteins secreted by Xanthomonas bacteria. The DNA binding domain contains a repeated, highly conserved 33-34 amino acid sequence, with the exception of the 12th and 13th amino acids. These two positions are highly variable, showing a strong correlation with specific nucleotide recognition, and can thus be engineered to bind to a target DNA sequence.

To produce a TALEN, a TALE protein is fused to a nuclease (N) comprising, for example, a wild-type or mutated Fokl endonuclease. The Fokl domain functions as a dimer, requiring two constructs with unique DNA binding domains for sites in the target genome with proper orientation and spacing. Specificity and off-target effect can be modulated by changing the number of amino acid residues between the TALE DNA binding domain and the Fokl cleavage domain and the number of bases between the two individual TALEN binding sites.

In some embodiments, the gene editing system is a zinc finger nuclease (ZFN) gene editing system. The zinc finger nuclease is an artificial nuclease which can be used to modify one or more nucleic acid sites of a target nucleic acid sequence. Similar to the TALEN editing system, a ZFN comprises a Fokl nuclease domain (or derivative thereof) fused to a DNA-binding domain. In the case of a ZFN, the DNA-binding domain comprises one or more zinc fingers. A zinc finger is a small protein structural motif stabilized by one or more zinc ions. A zinc finger can comprise, for example, Cys2His2, and can recognize an approximately 3-bp sequence. Various zinc fingers of known specificity can be combined to produce multi-finger polypeptides which recognize about 6, 9, 12, 15 or 18-bp sequences.

The ZFN recognizes non-palindromic DNA sites. To cleave the target site, a pair of ZFNs dimerizes and assembles to opposite strands of the target site. Various selection and modular assembly techniques are available to generate zinc fingers (and combinations thereof) recognizing specific sequences, including phage display, yeast one-hybrid systems, bacterial one-hybrid and two-hybrid systems, and mammalian cells.

In some embodiments, the gene editing system is a meganuclease gene editing system. A meganuclease is an artificial nuclease that recognize 15-40 base-pair cleavage sites. In some instances, meganucleases are grouped into families based on their structural motifs which affect nuclease activity and/or DNA recognition. Members of the LAGLIDADG family are characterized by having either one or two copies of the conserved LAGLIDADG motif. In some instances, the LAGLIDADG meganucleases with a single copy of the LAGLIDADG motif form homodimers, whereas members with two copies of the LAGLIDADG motif are found as monomers. The GIY-YIG family members have a GP-YIG module, which is 70-100 residues long and includes four or five conserved sequence motifs with four invariant residues, two of which are required for activity. The His-Cys box meganucleases are characterized by a highly conserved series of histidines and cysteines over a region encompassing several hundred amino acid residues. The NHN family, the members are defined by motifs containing two pairs of conserved histidines surrounded by asparagine residues. Strategies for engineering a meganuclease with altered DNA-binding specificity, e.g., to bind to a predetermined nucleic acid sequence are known in the art, and can be found in, e.g., Chevalier et al. (2002), Mol. Cell, 10:895-905; Epinat et al. (2003) Nucleic Acids Res 31: 2952-62; Silva et al. (2006) JMolBiol 361: 744-54; Seligman et al. (2002) Nucleic Acids Res 30: 3870-9; Sussman et al. (2004) JMol Biol 342: 31-41; Rosen et al. (2006) Nucleic Acids Res; Doyon et al. (2006) J. Am Chem Soc 128: 2477-84; Chen et al. (2009) Protein Eng Des Sel 22: 249-56; Arnould S (2006) JMolBiol. 355: 443-58; Smith (2006) Nucleic Acids Res. 363(2): 283-94.

In some instances, the meganuclease is a hybrid nuclease termed megaTAL comprising a TALE domain fused to the N-terminus of a meganuclease. In some cases, the meganuclease is a member of the LAGLIDADG family.

C. Homology-Directed Repair (HDR) Template

In certain embodiments, an unstimulated immune cell described herein is modified by a homology-directed repair mechanism. In some embodiments, a homology-directed repair (HDR) template comprising a polynucleotide encoding an antigen-binding polypeptide is transfected into a target immune cell and under conditions whereby the polynucleotide is inserted into the target site. In some embodiments, the HDR template comprises a 5′ homology arm homologous to a genomic sequence upstream, or 5′, to the target site and a 3′ homology arm homologous to the genomic sequence downstream, or 3′, to the target site. In some embodiments, the 5′ and/or 3′ homology arm is adjacent to the polynucleotide. In some embodiments, the 5′ homology arm is from about 50 to about 1000 nucleotides, about 50 nucleotides to about 500 nucleotides, from about 50 nucleotides to about 400 nucleotides, from about 50 to about 300 nucleotides, from about 50 nucleotides to about 200 nucleotides, from about 50 nucleotides to about 150 nucleotides, from about 100 nucleotides to about 500 nucleotides, from about 100 nucleotides to about 400 nucleotides, from about 100 nucleotides to about 300 nucleotides, from about 100 nucleotides to about 200 nucleotides, from about 200 nucleotides to about 500 nucleotides, from about 200 nucleotides to about 400 nucleotides, from about 200 nucleotides to about 300 nucleotides, from about 300 nucleotides to about 500 nucleotides, or from about 300 nucleotides to about 400 nucleotides in length. In some embodiments, the 5′ homology arm is about 50, about 60, about 70, about 80, about 90, about 100, about 120, about 140, about 150, about 160, about 180, about 200, about 220, about 240, about 250, about 260, about 280, about 300, about 350, about 400, about 450, about 500, about 600, about 700, about 800, about 900, or about 1000 nucleotides in length. In some instances, the 5′ homology arm is about 50, about 60, about 70, about 80, about 90, about 100, about 120, about 140, about 150, about 160, about 180, about 200, about 220, about 240, about 250, about 260, about 280, or about 300 nucleotides in length. In some instances, the 5′ homology arm is about 50, about 100, about 150, about 200, about 300, about 350, about 400, about 450, or about 500 nucleotides in length. In some embodiments, the 3′ homology arm is from about 50 to about 1000 nucleotides, from about 50 nucleotides to about 500 nucleotides, from about 50 nucleotides to about 400 nucleotides, from about 50 to about 300 nucleotides, from about 50 nucleotides to about 200 nucleotides, from about 50 nucleotides to about 150 nucleotides, from about 100 nucleotides to about 500 nucleotides, from about 100 nucleotides to about 400 nucleotides, from about 100 nucleotides to about 300 nucleotides, from about 100 nucleotides to about 200 nucleotides, from about 200 nucleotides to about 500 nucleotides, from about 200 nucleotides to about 400 nucleotides, from about 200 nucleotides to about 300 nucleotides, from about 300 nucleotides to about 500 nucleotides, or from about 300 nucleotides to about 400 nucleotides in length. In some embodiments, the 3′ homology arm is about 50, about 60, about 70, about 80, about 90, about 100, about 120, about 140, about 150, about 160, about 180, about 200, about 220, about 240, about 250, about 260, about 280, about 300, about 350, about 400, about 450, about 500, about 600, about 700, about 800, about 900, or about 1000 nucleotides in length. In some instances, the 3′ homology arm is about 50, about 60, about 70, about 80, about 90, about 100, about 120, about 140, about 150, about 160, about 180, about 200, about 220, about 240, about 250, about 260, about 280, or about 300 nucleotides in length. In some instances, the 3′ homology arm is about 50, about 100, about 150, about 200, about 300, about 350, about 400, about 450, or about 500 nucleotides in length. In some embodiments, the length of the HDR template is from about 2 kilo-base pairs (kb) to about 5 kb, from about 2.3 kb to about 5 kb, from about 3 kb to about 5 kb, from about 3 kb to about 4 kb, from about 2 kb to about 4 kb, from about 2.3 kb to about 4 kb, from about 2 kb to about 3 kb, from about 2.3 kb to about 3 kb, or from about 4 kb to about 5 kb. In some instances, the length of the HDR template is about 2 kb, about 2.3 kb, about 2.5 kb, about 3 kb, about 4 kb, or about 5 kb. In some cases, the HDR template is also referred to herein as an ultramer. In some cases, the antigen-binding polypeptide is an antigen receptor, optionally a CAR or a TCR.

In some embodiments, the HDR template is a double-stranded DNA (dsDNA) template. In some embodiments, the dsDNA template comprises natural nucleotides, modified nucleotides, or a combination thereof. In some embodiments, the dsDNA template comprises a 5′ homology arm homologous to a genomic sequence upstream, or 5′, to the target site and a 3′ homology arm homologous to the genomic sequence downstream, or 3′, to the target site. In some embodiments, the 5′ and/or 3′ homology arm is adjacent to the polynucleotide. In some embodiments, the 5′ homology arm is from about 50 to about 1000 nucleotides, from about 50 nucleotides to about 500 nucleotides, from about 50 nucleotides to about 400 nucleotides, from about 50 to about 300 nucleotides, from about 50 nucleotides to about 200 nucleotides, from about 50 nucleotides to about 150 nucleotides, from about 100 nucleotides to about 500 nucleotides, from about 100 nucleotides to about 400 nucleotides, from about 100 nucleotides to about 300 nucleotides, from about 100 nucleotides to about 200 nucleotides, from about 200 nucleotides to about 500 nucleotides, from about 200 nucleotides to about 400 nucleotides, from about 200 nucleotides to about 300 nucleotides, from about 300 nucleotides to about 500 nucleotides, or from about 300 nucleotides to about 400 nucleotides in length. In some embodiments, the 5′ homology arm is about 50, about 60, about 70, about 80, about 90, about 100, about 120, about 140, about 150, about 160, about 180, about 200, about 220, about 240, about 250, about 260, about 280, about 300, about 350, about 400, about 450, about 500, about 600, about 700, about 800, about 900, or about 1000 nucleotides in length. In some instances, the 5′ homology arm is about 50, about 60, about 70, about 80, about 90, about 100, about 120, about 140, about 150, about 160, about 180, about 200, about 220, about 240, about 250, about 260, about 280, or about 300 nucleotides in length. In some instances, the 5′ homology arm is about 50, about 100, about 150, about 200, about 300, about 350, about 400, about 450, or about 500 nucleotides in length. In some embodiments, the 3′ homology arm is from about 50 to about 1000 nucleotides, from about 50 nucleotides to about 500 nucleotides, from about 50 nucleotides to about 400 nucleotides, from about 50 to about 300 nucleotides, from about 50 nucleotides to about 200 nucleotides, from about 50 nucleotides to about 150 nucleotides, from about 100 nucleotides to about 500 nucleotides, from about 100 nucleotides to about 400 nucleotides, from about 100 nucleotides to about 300 nucleotides, from about 100 nucleotides to about 200 nucleotides, from about 200 nucleotides to about 500 nucleotides, from about 200 nucleotides to about 400 nucleotides, from about 200 nucleotides to about 300 nucleotides, from about 300 nucleotides to about 500 nucleotides, or from about 300 nucleotides to about 400 nucleotides in length. In some embodiments, the 3′ homology arm is about 50, about 60, about 70, about 80, about 90, about 100, about 120, about 140, about 150, about 160, about 180, about 200, about 220, about 240, about 250, about 260, about 280, about 300, about 350, about 400, about 450, about 500, about 600, about 700, about 800, about 900, or about 1000 nucleotides in length. In some instances, the 3′ homology arm is about 50, about 60, about 70, about 80, about 90, about 100, about 120, about 140, about 150, about 160, about 180, about 200, about 220, about 240, about 250, about 260, about 280, or about 300 nucleotides in length. In some instances, the 3′ homology arm is about 50, about 100, about 150, about 200, about 300, about 350, about 400, about 450, or about 500 nucleotides in length. In some embodiments, the length of the dsDNA template is from about 2 kb to about 5 kb, from about 2.3 kb to about 5 kb, from about 3 kb to about 5 kb, from about 3 kb to about 4 kb, from about 2 kb to about 4 kb, from about 2.3 kb to about 4 kb, from about 2 kb to about 3 kb, from about 2.3 kb to about 3 kb, or from about 4 kb to about 5 kb. In some instances, the length of the dsDNA template is about 2 kb, about 2.3 kb, about 2.5 kb, about 3 kb, about 4 kb, or about 5 kb.

In some embodiments, the HDR template is a single-stranded DNA (ssDNA) template. In some embodiments, the ssDNA template comprises natural nucleotides, modified nucleotides, or a combination thereof. In some embodiments, the ssDNA template comprises a 5′ homology arm homologous to a genomic sequence upstream, or 5′, to the target site and a 3′ homology arm homologous to the genomic sequence downstream, or 3′, to the target site. In some embodiments, the 5′ and/or 3′ homology arm is adjacent to the polynucleotide. In some embodiments, the 5′ homology arm is from about 50 to about 1000 nucleotides, from about 50 nucleotides to about 500 nucleotides, from about 50 nucleotides to about 400 nucleotides, from about 50 to about 300 nucleotides, from about 50 nucleotides to about 200 nucleotides, from about 50 nucleotides to about 150 nucleotides, from about 100 nucleotides to about 500 nucleotides, from about 100 nucleotides to about 400 nucleotides, from about 100 nucleotides to about 300 nucleotides, from about 100 nucleotides to about 200 nucleotides, from about 200 nucleotides to about 500 nucleotides, from about 200 nucleotides to about 400 nucleotides, from about 200 nucleotides to about 300 nucleotides, from about 300 nucleotides to about 500 nucleotides, or from about 300 nucleotides to about 400 nucleotides in length. In some embodiments, the 5′ homology arm is about 50, about 60, about 70, about 80, about 90, about 100, about 120, about 140, about 150, about 160, about 180, about 200, about 220, about 240, about 250, about 260, about 280, about 300, about 350, about 400, about 450, about 500, about 600, about 700, about 800, about 900, or about 1000 nucleotides in length. In some instances, the 5′ homology arm is about 50, about 60, about 70, about 80, about 90, about 100, about 120, about 140, about 150, about 160, about 180, about 200, about 220, about 240, about 250, about 260, about 280, or about 300 nucleotides in length. In some instances, the 5′ homology arm is about 50, about 100, about 150, about 200, about 300, about 350, about 400, about 450, or about 500 nucleotides in length. In some embodiments, the 3′ homology arm is from about 50 to about 1000 nucleotides, from about 50 nucleotides to about 500 nucleotides, from about 50 nucleotides to about 400 nucleotides, from about 50 to about 300 nucleotides, from about 50 nucleotides to about 200 nucleotides, from about 50 nucleotides to about 150 nucleotides, from about 100 nucleotides to about 500 nucleotides, from about 100 nucleotides to about 400 nucleotides, from about 100 nucleotides to about 300 nucleotides, from about 100 nucleotides to about 200 nucleotides, from about 200 nucleotides to about 500 nucleotides, from about 200 nucleotides to about 400 nucleotides, from about 200 nucleotides to about 300 nucleotides, from about 300 nucleotides to about 500 nucleotides, or from about 300 nucleotides to about 400 nucleotides in length. In some embodiments, the 3′ homology arm is about 50, about 60, about 70, about 80, about 90, about 100, about 120, about 140, about 150, about 160, about 180, about 200, about 220, about 240, about 250, about 260, about 280, about 300, about 350, about 400, about 450, about 500, about 600, about 700, about 800, about 900, or about 1000 nucleotides in length. In some instances, the 3′ homology arm is about 50, about 60, about 70, about 80, about 90, about 100, about 120, about 140, about 150, about 160, about 180, about 200, about 220, about 240, about 250, about 260, about 280, or about 300 nucleotides in length. In some instances, the 3′ homology arm is about 50, about 100, about 150, about 200, about 300, about 350, about 400, about 450, or about 500 nucleotides in length. In some embodiments, the length of the ssDNA template is from about 2 kb to about 5 kb, from about 2.3 kb to about 5 kb, from about 3 kb to about 5 kb, from about 3 kb to about 4 kb, from about 2 kb to about 4 kb, from about 2.3 kb to about 4 kb, from about 2 kb to about 3 kb, from about 2.3 kb to about 3 kb, or from about 4 kb to about 5 kb. In some instances, the length of the ssDNA template is about 2 kb, about 2.3 kb, about 2.5 kb, about 3 kb, about 4 kb, or about 5 kb.

In some embodiments, the HDR template is inserted in a plasmid prior to introduced into an immune cell. Exemplary plasmids include, but are not limited to, pBAD/His, pCal-n, pET-3a-c, pET32a-c, pGEX-2T, pTriEx-1, pUC19, pAd5F35, pAdDeltaF6, or TLCV2.

D. Transfection

In some embodiments, the gene-editing system and the HDR template are introduced into an unstimulated immune cell through a non-viral delivery method. In non-viral delivery methods, the nucleic acid can be naked DNA or in a non-viral plasmid or vector. Exemplary non-viral delivery methods include, but are not limited to, electroporation, a cell squeezing method, calcium phosphate, heat shock, liposomal delivery, cationic polymers, lipid-polymers, cell-penetrating peptides, cationic nanocarriers, hydrodynamic delivery, ultrasound, cationic lipids, nanoparticles, ballistic DNA injection, magneto-fection, and photo-poration.

In some embodiments, the gene-editing system and the HDR template are introduced into an unstimulated immune cell by electroporation. In some instances, the gene-editing system (e.g., the endonuclease and/or the guide nucleic acid) and the HDR template are introduced into an unstimulated immune cell simultaneously by electroporation. In other instances, the gene-editing system (e.g., the endonuclease and/or the guide nucleic acid) is introduced into the unstimulated immune cell simultaneously by electroporation first followed by a separate electroporation of the HDR template; or alternatively, the HDR template is introduced into the unstimulated immune cell simultaneously by electroporation first followed by a separate electroporation of the gene-editing system (e.g., the endonuclease and/or the guide nucleic acid). In additional instances, the endonuclease, the guide nucleic acid, and the HDR template are sequentially introduced into the unstimulated immune cell by electroporation.

In some embodiments, the endonuclease is introduced into an unstimulated immune cell at a concentration of from about 1 pM to about 10 mM. In some instances, the concentration of the endonuclease is from about 1 pM to about 5 mM, about 1 pM to about 3 mM, about 1 pM to about 1 mM, about 1 pM to about 0.8 mM, about 1 pM to about 0.5 mM, about 1 pM to about 0.2 mM, about 1 pM to about 0.1 mM, about 1 pM to about 0.09 mM, about 1 pM to about 0.05 mM, about 1 pM to about 0.01 mM, about 1 pM to about 5 pM, about 1 pM to about 1 pM, about 1 pM to about 500 nM, about 1 pM to about 100 nM, about 1 nM to about 10 mM, about 1 nM to about 5 mM, about 1 nM to about 3 mM, about 1 nM to about 1 mM, about 1 nM to about 0.8 mM, about 1 nM to about 0.5 mM, about 1 nM to about 0.2 mM, about 1 nM to about 0.1 mM, about 1 nM to about 0.09 mM, about 1 nM to about 0.05 mM, about 1 nM to about 0.01 mM, about 1 nM to about 5 pM, about 1 nM to about 1 pM, about 1 nM to about 500 nM, about 1p M to about 10 mM, about 1 pM to about 5 mM, about 1 pM to about 3 mM, about 1 pM to about 1 mM, about 1 pM to about 0.8 mM, about 1 pM to about 0.5 mM, about 1 pM to about 0.2 mM, about 1 pM to about 0.1 mM, about 1 pM to about 0.09 mM, about 1 pM to about 0.05 mM, about 1 pM to about 0.01 mM, or about 1 pM to about 5 pM.

In some embodiments, the guide nucleic acid (e.g., the guide RNA molecule) is introduced into an unstimulated immune cell at a concentration of from about 1 pM to about 10 mM. In some instances, the concentration of the guide nucleic acid (e.g., the guide RNA molecule) is from about 1 pM to about 5 mM, about 1 pM to about 3 mM, about 1 pM to about 1 mM, about 1 pM to about 0.8 mM, about 1 pM to about 0.5 mM, about 1 pM to about 0.2 mM, about 1 pM to about 0.1 mM, about 1 pM to about 0.09 mM, about 1 pM to about 0.05 mM, about 1 pM to about 0.01 mM, about 1 pM to about 5 pM, about 1 pM to about 1 pM, about 1 pM to about 500 nM, about 1 pM to about 100 nM, about 1 nM to about 10 mM, about 1 nM to about 5 mM, about 1 nM to about 3 mM, about 1 nM to about 1 mM, about 1 nM to about 0.8 mM, about 1 nM to about 0.5 mM, about 1 nM to about 0.2 mM, about 1 nM to about 0.1 mM, about 1 nM to about 0.09 mM, about 1 nM to about 0.05 mM, about 1 nM to about 0.01 mM, about 1 nM to about 5 pM, about 1 nM to about 1 pM, about 1 nM to about 500 nM, about 1 pM to about 10 mM, about 1 μM to about 5 mM, about 1 μM to about 3 mM, about 1 μM to about 1 mM, about 1 μM to about 0.8 mM, about 1 μM to about 0.5 mM, about 1 μM to about 0.2 mM, about 1 μM to about 0.1 mM, about 1 μM to about 0.09 mM, about 1 μM to about 0.05 mM, about 1 μM to about 0.01 mM, or about 1 μM to about 5 μM.

In some embodiments, the HDR template is introduced into an unstimulated immune cell at a concentration of from about 1 pM to about 10 mM. In some instances, the concentration of the HDR template is from about 1 pM to about 5 mM, about 1 pM to about 3 mM, about 1 pM to about 1 mM, about 1 pM to about 0.8 mM, about 1 pM to about 0.5 mM, about 1 pM to about 0.2 mM, about 1 pM to about 0.1 mM, about 1 pM to about 0.09 mM, about 1 pM to about 0.05 mM, about 1 pM to about 0.01 mM, about 1 pM to about 5 pM, about 1 pM to about 1 pM, about 1 pM to about 500 nM, about 1 pM to about 100 nM, about 1 nM to about 10 mM, about 1 nM to about 5 mM, about 1 nM to about 3 mM, about 1 nM to about 1 mM, about 1 nM to about 0.8 mM, about 1 nM to about 0.5 mM, about 1 nM to about 0.2 mM, about 1 nM to about 0.1 mM, about 1 nM to about 0.09 mM, about 1 nM to about 0.05 mM, about 1 nM to about 0.01 mM, about 1 nM to about 5 μM, about 1 nM to about 1 μM, about 1 nM to about 500 nM, about 1 μM to about 10 mM, about 1 μM to about 5 mM, about 1 μM to about 3 mM, about 1 μM to about 1 mM, about 1 μM to about 0.8 mM, about 1 μM to about 0.5 mM, about 1 μM to about 0.2 mM, about 1 μM to about 0.1 mM, about 1 μM to about 0.09 mM, about 1 μM to about 0.05 mM, about 1 μM to about 0.01 mM, or about 1 μM to about 5 μM.

In some embodiments, the gene-editing system is a CRISPR-Cas system. In some instances, a Cas (e.g., a Cas9) endonuclease is introduced into an unstimulated immune cell at a concentration of from about 1 pM to about 10 mM. In some instances, the concentration of the Cas (e.g., a Cas9) endonuclease is from about 1 pM to about 5 mM, about 1 pM to about 3 mM, about 1 pM to about 1 mM, about 1 pM to about 0.8 mM, about 1 pM to about 0.5 mM, about 1 pM to about 0.2 mM, about 1 pM to about 0.1 mM, about 1 pM to about 0.09 mM, about 1 pM to about 0.05 mM, about 1 pM to about 0.01 mM, about 1 pM to about 5 pM, about 1 pM to about 1 μM, about 1 μM to about 500 nM, about 1 μM to about 100 nM, about 1 nM to about 10 mM, about 1 nM to about 5 mM, about 1 nM to about 3 mM, about 1 nM to about 1 mM, about 1 nM to about 0.8 mM, about 1 nM to about 0.5 mM, about 1 nM to about 0.2 mM, about 1 nM to about 0.1 mM, about 1 nM to about 0.09 mM, about 1 nM to about 0.05 mM, about 1 nM to about 0.01 mM, about 1 nM to about 5 μM, about 1 nM to about 1 μM, about 1 nM to about 500 nM, about 1 μM to about 10 mM, about 1 μM to about 5 mM, about 1 μM to about 3 mM, about 1 μM to about 1 mM, about 1 μM to about 0.8 mM, about 1 μM to about 0.5 mM, about 1 μM to about 0.2 mM, about 1 μM to about 0.1 mM, about 1 μM to about 0.09 mM, about 1 μM to about 0.05 mM, about 1 μM to about 0.01 mM, or about 1 μM to about 5 μM.

In some embodiments, a ratio of the Cas (e.g., Cas9) endonuclease concentration and the guide nucleic acid concentration introduced into an unstimulated immune cell is about 1:1, 1:2, 1:3, 1:4, 1:5, 1:6, 1:7, 1:8, 1:9, 1:10, 2:1, 3:1, 4:1, 5:1, 6:1, 7:1, 8:1, 9:1, or 10:1. In some instances, the ratio of the Cas endonuclease and the guide nucleic acid is about 1:1. In some instances, the ratio of the Cas endonuclease and the guide nucleic acid is about 1:2. In some instances, the ratio of the Cas endonuclease and the guide nucleic acid is about 1:3. In some instances, the ratio of the Cas endonuclease and the guide nucleic acid is about 2:1. In some instances, the ratio of the Cas endonuclease and the guide nucleic acid is about 3:1.

In some embodiments, a ratio of the CRISPR-Cas system and the HDR template introduced into an unstimulated immune cell is about 1:1, about 1:2, about 1:3, about 1:4, about 1:5, about 1:6, about 1:7, about 1:8, about 1:9, about 1:10, about 1:12, about 1:15, about 1:18, about 1:20, about 1:25, about 1:30, about 1:35, about 1:40, about 2:1, about 3:1, about 4:1, about 5:1, about 6:1, about 7:1, about 8:1, about 9:1, about 10:1, about 12:1, about 15:1, about 20:1, about 30:1, or about 40:1. In some instances, the ratio of the CRISPR-Cas system and the HDR template is about 1:1. In some instances, the ratio of the CRISPR-Cas system and the HDR template is about 1:2. In some instances, the ratio of the CRISPR-Cas system and the HDR template is about 2:1.

In some embodiments, a ratio of the gene-editing nuclease (e.g., the endonuclease) and the HDR template introduced into an unstimulated immune cell is about 1:1, about 1:2, about 1:3, about 1:4, about 1:5, about 1:6, about 1:7, about 1:8, about 1:9, about 1:10, about 2:1, about 3:1, about 4:1, about 5:1, about 6:1, about 7:1, about 8:1, about 9:1, or about 10:1. In some instances, the ratio of the gene-editing nuclease (e.g., the endonuclease) and the HDR template is about 1:1. In some instances, the ratio of the gene-editing nuclease (e.g., the endonuclease) and the HDR template is about 1:2. In some instances, the ratio of the gene-editing nuclease (e.g., the endonuclease) and the HDR template is about 2:1.

In some embodiments, the electrophoresis is performed at a temperature of from about 2° C. to about 40° C. In some instances, the electrophoresis is performed at a temperature of from about 2° C. to about 37° C., about 2° C. to about 35° C., about 2° C. to about 33° C., about 2° C. to about 30° C., about 2° C. to about 28° C., about 2° C. to about 26° C., about 2° C. to about 25° C., about 2° C. to about 20° C., about 2° C. to about 18° C., about 2° C. to about 15° C., about 2° C. to about 10° C., about 2° C. to about 8° C., about 4° C. to about 37° C., about 4° C. to about 35° C., about 4° C. to about 33° C., about 4° C. to about 30° C., about 4° C. to about 28° C., about 4° C. to about 26° C., about 4° C. to about 25° C., about 4° C. to about 20° C., about 4° C. to about 18° C., about 4° C. to about 15° C., about 4° C. to about 10° C., about 4° C. to about 8° C., about 20° C. to about 37° C., about 20° C. to about 35° C., about 20° C. to about 33° C., about 20° C. to about 30° C., about 20° C. to about 28° C., about 20° C. to about 26° C., about 20° C. to about 25° C., about 22° C. to about 25° C., about 22° C. to about 28° C., about 24° C. to about 30° C., about 24° C. to about 28° C., or about 25° C. to about 30° C. In some cases, the electrophoresis is performed at a temperature of from about 20° C. to about 30° C. In some cases, the electrophoresis is performed at a temperature of from about 25° C. to about 30° C. In some cases, the electrophoresis is performed at a temperature of from about 25° C. to about 28° C.

Exemplary electroporation technologies include, but are not limited to, the NUCLEOFECTOR™ platform from Lonza, the Amaxa NUCLEOFECTOR™ II Electroporation machine from Lonza, the FLOW ELECTROPORATION® technology from MaxCyte, and the Gene Pulser MXCELL™ electroporation system from Bio-Rad. A skilled artisan would understand that the pulse type, duration of pulsing, voltage, and frequency of use are dependent upon the type of instrument and the cell type; and that optimization of the efficiency of the transfection can be modulated based on the pulse type, duration of pulsing, voltage, frequency of use, and concentrations of the endonuclease, guide nucleic acid, and/or the HDR template.

In some embodiments, the HDR template facilitates HDR at the target site in about 10% or higher of the population of unstimulated immune cells. In some instances, the HDR template facilitates HDR at the target site in about 20% or higher, 30% or higher, 40% or higher, 50% or higher, 60% or higher, 70% or higher, 80% or higher, 90% or higher, or 95% or higher of the population of unstimulated immune cells.

In some embodiments, the efficiency of the transfection method is from about 2% to about 99%, from about 5% to about 99%, from about 8% to about 99%, from about 10% to about 99%, from about 12% to about 99%, from about 13% to about 99%, from about 15% to about 99%, from about 20% to about 99%, from 30% to about 99%, from about 40% to about 99%, from about 50% to about 99%, from about 60% to about 99%, from about 70% to about 99%, from about 2% to about 80%, from about 5% to about 80%, from about 8% to about 80%, from about 10% to about 80%, from about 12% to about 80%, from about 13% to about 80%, from about 15% to about 80%, from about 20% to about 80%, from about 30% to about 80%, from about 40% to about 80%, from about 50% to about 80%, from about 20% to about 70%, or from about 30% to about 60%. In some instances, the efficiency is about 2%, about 5%, about 8%, about 10%, about 12%, about 13%, about 15%, about 18%, about 20%, about 25%, about 30%, about 35%, about 40%, about 45%, about 50%, about 60%, about 70%, about 80%, about 90%, about 95%, or about 99%.

E. Modified Unstimulated Immune Cells

In some embodiments, the unstimulated immune cells after non-viral delivery of the gene-editing system and the HDR template are cultured under a non-expansion condition for up to and about 72 hours to generate modified unstimulated immune cells. In some instances, the unstimulated immune cells are cultured from about 18 hours to about 72 hours, from about 18 hours to about 36 hours, from about 18 hours to about 24 hours, from about 24 hours to about 72 hours, from about 24 hours to about 36 hours, or from about 36 hours to about 72 hours to generate modified unstimulated immune cells. In some instances, the unstimulated immune cells are cultured for about 48 hours or less, about 36 hours or less, about 24 hours or less, or about 18 hours or less to generate modified unstimulated immune cells. In some instances, the unstimulated immune cells are cultured for about 18 hours, 24 hours, 36 hours, 48 hours, or about 72 hours to generate modified unstimulated immune cells. In some cases, the unstimulated immune cells are cultured in a culture vessel (e.g., a culture bag or a bioreactor) at about 37° C. In some cases, the culture vessel comprises about 90%, about 95%, or about 99% humidity. In some cases, the culture vessel comprises about 0.5% carbon dioxide.

In some embodiments, the cell media used with the modified unstimulated immune cells comprises a complete media, a chemically-defined media, or a fetal bovine serum (FBS)-based media, e.g., comprising from about 1% to about 10% FBS. In some instances, the cell media is a minimum media. Exemplary media for culturing the modified unstimulated immune cells include, but are not limited to, CliniMACS buffer from Miltenyi Biotech and CTS™ OPTMIZER™ from ThermoFisher.

In some instances, the cell media further comprises a protein that coats the inner surface of the culture vessel without interacting with the cultured modified unstimulated immune cells. In some cases, the media comprises from about 0.1% to about 5% w/v or from about 0.5% to about 2% w/v of the protein. In some cases, the media comprises about 0.1%, about 0.2%, about 0.3%, about 0.4%, about 0.5%, about 0.6%, about 0.7%, about 0.8%, about 0.9%, or about 1% w/v of the protein. In some cases, the protein is an isolated recombinant protein or a fragment thereof. In some cases, the protein is an engineered de novo polypeptide. In some cases, the protein is a naturally-occurring protein or fragment thereof. In some cases, the protein is albumin (e.g., human serum albumin or HSA).

In some embodiments, the cell media comprises from about 0.1% to about 5% w/v or from about 0.5% to about 2% w/v of albumin (e.g., HSA). In some instances, the media comprises about 0.1%, about 0.2%, about 0.3%, about 0.4%, about 0.5%, about 0.6%, about 0.7%, about 0.8%, about 0.9%, or about 1% w/v of albumin (e.g., HSA). In some cases, the albumin (e.g., HSA) is a full-length albumin. In other cases, the albumin (e.g., HSA) is a fragment thereof, e.g., without the signaling peptide.

In some embodiments, the population of modified unstimulated immune cells generated by the above described method comprises about 5 million cells, 10 million cells, 20 million cells, 50 million cells, 100 million cells, 500 million cells, 1 billion cells, 2 billion cells, 3 billion cells, 4 billion cells, 5 billion cells, 10 billion cells, or more. In some cases, the population of modified unstimulated immune cells generated by the above described method comprises from about 50 million cells to about 5 billion cells. In some cases, the population of modified unstimulated immune cells generated by the above described method comprises from about 50 million cells to about 4 billion cells.

F. Cryopreservation

In some embodiments, the modified unstimulated immune cells are cryopreserved after culturing for up to or about 72 hours. In some instances, the modified unstimulated immune cells are harvested from the culture vessel and resuspended is a cryopreservation media. In some instances, the cryopreservation media comprises a CRYOSTOR® CSB media (BioLife Solutions) and DMSO. In some instances, the DMSO is a 2%, 5%, or 10% v/v solution. In some instances, the DMSO is CRYOSTOR® CS5 or CRYOSTOR® CS10 (BioLife Solutions).

In some cases, the final concentration of the DMSO is from about 0% v/v to about 7.5% v/v. In some cases, the final concentration of the DMSO is about 2.5% v/v. In some cases, the final concentration of the DMSO is about 5% v/v. In some cases, the temperature during resuspension is from about 24° C. to about 28° C.

Upon resuspension in the cryopreservation media, the modified unstimulated immune cells are cryofrozen, in which the temperature of the cells is decreased from the resuspension temperature to about −80° C. in a step-wise method. In some instances, a decrease of from about 1-5 degrees Celsius per minute is used in the step-wise method. In some instances, a decrease of about 1 degree Celsius per minute is used in the step-wise method. In some instances, the step-wise method comprises reducing the temperature from the resuspension temperature to about −80° C. within about 30 minutes to about 3 hours. In some cases, the resuspension temperature is from about 24° C. to about 28° C., from about 26° C. to about 28° C., or from about 24° C. to about 26° C.

In some embodiments, the method described herein that generates cryofrozen modified unstimulated immune cells further provides a cell viability of from about 10% to about 99%, from about 12% to about 99%, from about 13% to about 99%, from about 15% to about 99%, from about 20% to about 99%, from 30% to about 99%, from about 40% to about 99%, from about 50% to about 99%, from about 60% to about 99%, from about 70% to about 99%, from about 10% to about 80%, from about 12% to about 80%, from about 13% to about 80%, from about 15% to about 80%, from about 20% to about 80%, from about 30% to about 80%, from about 40% to about 80%, from about 50% to about 80%, from about 20% to about 70%, or from about 30% to about 60%. In some cases, the method provides a cell viability of about 10%, about 12%, about 13%, about 15%, about 18%, about 20%, about 25%, about 30%, about 35%, about 40%, about 45%, about 50%, about 60%, about 70%, about 80%, about 90%, about 95%, or about 99%.

G. Modified Stimulated Immune Cells

In some embodiments, the modified unstimulated immune cells are optionally stimulated prior to cryopreservation. In some instances, the modified unstimulated immune cells are stimulated and expanded from about 5 days to about 12 days. In some cases, the modified unstimulated immune cells are stimulated and expanded for about 5 days, about 6 days, about 7 days, about 8 days, about 9 days, about 10 days, about 11 days, about 12 days, or more. In some cases, the modified unstimulated immune cells are stimulated and expanded for about 10 days up to about 12 days.

In some embodiments, the modified unstimulated immune cells are incubated in the presence of an agent capable of stimulating the unstimulated immune cell. In some instances where the unstimulated immune cells are T cells, the agent is capable of activating one or more intracellular signaling domains of one or more components of a TCR complex, such as a CD3 zeta chain, or capable of activating signaling through such complex or component. In some instances, the agent is an anti-CD3 antibody, an anti-CD28 antibody, an anti-4-1BB antibody, or a cytokine such as IL-2, IL-15, IL-7, or IL-21, or artificial antigen-present cells (aAPCs). In some instances, the antibody is further coupled to or present on the surface of a solid support, such as a bead. In some cases, the agent is Dynabeads human T-Activator CD3/CD28 (ThermoFisher), optionally at a 3:1 bead to cell ratio; or MACS® GEMP T cell TRANSACT™ (Mitenyi Biotec).

In some cases, the population of modified stimulated immune cells generated by the method described herein comprises about 5 million cells, about 10 million cells, about 20 million cells, about 50 million cells, about 100 million cells, about 500 million cells, about 1 billion cells, about 5 billion cells, about 10 billion cells, about 20 billion cells, or more.

In some embodiments, the method described herein that generates cryofrozen modified stimulated immune cells further provides a cell viability of from about 10% to about 99%, from about 12% to about 99%, from about 13% to about 99%, from about 15% to about 99%, from about 20% to about 99%, from 30% to about 99%, from about 40% to about 99%, from about 50% to about 99%, from about 60% to about 99%, from about 70% to about 99%, from about 10% to about 80%, from about 12% to about 80%, from about 13% to about 80%, from about 15% to about 80%, from about 20% to about 80%, from about 30% to about 80%, from about 40% to about 80%, from about 50% to about 80%, from about 20% to about 70%, or from about 30% to about 60%. In some cases, the method provides a cell viability of about 10%, about 12%, about 13%, about 15%, about 18%, about 20%, about 25%, about 30%, about 35%, about 40%, about 45%, about 50%, about 60%, about 70%, about 80%, about 90%, about 95%, or about 99%.

### III. Antigen-Binding Polypeptides

In certain embodiments, disclosed herein is a modified unstimulated immune cell or a modified stimulated immune cell which expresses an exogenous antigen-binding polypeptide. In some instances, the antigen-binding polypeptide is a chimeric antigen receptor (CAR). In other instances, the antigen-binding polypeptide is a T-cell receptor (TCR). In additional instances, the antigen-binding polypeptide is an antigen-binding domain, a cell surface receptor ligand, or a polypeptide that binds to a tumor antigen.

A. Chimeric Antigen Receptors

In some embodiments, the modified unstimulated immune cell or modified stimulated immune cell, e.g., modified unstimulated or stimulated T cell, expresses a chimeric antigen receptor (CAR). CARs of the present invention comprise an antigen binding domain, a transmembrane domain, a hinge domain, and an intracellular signaling domain. Any modified cell comprising a CAR comprising any antigen binding domain, any hinge, any transmembrane domain, any intracellular costimulatory domain, and any intracellular signaling domain described herein is envisioned, and can readily be understood and made by a person of skill in the art in view of the disclosure herein.

The antigen binding domain may be operably linked to another domain of the CAR, such as the transmembrane domain or the intracellular domain, both described elsewhere herein, for expression in the cell. In one embodiment, a first nucleic acid sequence encoding the antigen binding domain is operably linked to a second nucleic acid encoding a transmembrane domain, and further operably linked to a third a nucleic acid sequence encoding an intracellular domain.

The antigen binding domains described herein can be combined with any of the transmembrane domains described herein, any of the intracellular domains or cytoplasmic domains described herein, or any of the other domains described herein that may be included in a CAR of the present invention. A subject CAR of the present invention may also include a spacer domain as described herein. In some embodiments, each of the antigen binding domain, transmembrane domain, and intracellular domain is separated by a linker.

1. Antigen Binding Domain

The antigen binding domain of a CAR is an extracellular region of the CAR for binding to a specific target antigen including proteins, carbohydrates, and glycolipids. In some embodiments, the CAR comprises affinity to a target antigen (e.g. a tumor associated antigen) on a target cell (e.g. a cancer cell). The target antigen may include any type of protein, or epitope thereof, associated with the target cell. For example, the CAR may comprise affinity to a target antigen on a target cell that indicates a particular status of the target cell.

As described herein, a CAR of the present disclosure having affinity for a specific target antigen on a target cell may comprise a target-specific binding domain. In some embodiments, the target-specific binding domain is a murine target-specific binding domain, e.g., the target-specific binding domain is of murine origin. In some embodiments, the target-specific binding domain is a human target-specific binding domain, e.g., the target-specific binding domain is of human origin.

The antigen binding domain can include any domain that binds to the antigen and may include, but is not limited to, a monoclonal antibody, a polyclonal antibody, a synthetic antibody, a human antibody, a humanized antibody, a non-human antibody, and any fragment thereof. Thus, in one embodiment, the antigen binding domain portion comprises a mammalian antibody or a fragment thereof. In some embodiments, the antigen binding domain comprises a full-length antibody. In some embodiments, the antigen binding domain comprises an antigen binding fragment (Fab), e.g., Fab, Fab′, F(ab′)₂, a monospecific Fab₂, a bispecific Fab₂, a trispecific Fab₂, a single-chain variable fragment (scFv), dAb, tandem scFv, VhH, V-NAR, camelid, diabody, minibody, triabody, or tetrabody.

In some embodiments, a CAR of the present disclosure may have affinity for one or more target antigens on one or more target cells. In some embodiments, a CAR may have affinity for one or more target antigens on a single target cell. In such embodiments, the CAR is a bispecific CAR, or a multispecific CAR. In some embodiments, the CAR comprises one or more target-specific binding domains that confer affinity for one or more target antigens. In some embodiments, the CAR comprises one or more target-specific binding domains that confer affinity for the same target antigen. For example, a CAR comprising one or more target-specific binding domains having affinity for the same target antigen could bind distinct epitopes of the target antigen. When a plurality of target-specific binding domains is present in a CAR, the binding domains may be arranged in tandem and may be separated by linker peptides. For example, in a CAR comprising two target-specific binding domains, the binding domains are connected to each other covalently on a single polypeptide chain, through a polypeptide linker, an Fc hinge region, or a membrane hinge region.

As used herein, the term “single-chain variable fragment” or “scFv” is a fusion protein of the variable regions of the heavy (VH) and light chains (VL) of an immunoglobulin (e.g., mouse or human) covalently linked to form a VH::VL heterodimer. The heavy (VH) and light chains (VL) are either joined directly or joined by a peptide-encoding linker or spacer, which connects the N-terminus of the VH with the C-terminus of the VL, or the C-terminus of the VH with the N-terminus of the VL. The terms “linker” and “spacer” are used interchangeably herein. In some embodiments, the antigen binding domain (e.g., Tn-MUC1 binding domain) comprises an scFv having the configuration from N-terminus to C-terminus, VH-linker-VL. In some embodiments, the antigen binding domain (e.g., a Tn-MUC1 binding domain, a PSMA binding domain) comprises an scFv having the configuration from N-terminus to C-terminus, VL-linker-VH. Those of skill in the art would be able to select the appropriate configuration for use in the present invention.

The linker is typically rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80(6):1910-1917 (2008) and WO 2014/087010, the contents of which are hereby incorporated by reference in their entireties. Various linker sequences are known in the art, including, without limitation, glycine serine (GS) linkers such as (GS)n, (GSGGS)n, (GGGS)n, and (GGGGS)n, where n represents an integer of at least 1. Exemplary linker sequences can comprise amino acid sequences including, without limitation, GGSG, GGSGG, GSGSG, GSGGG, GGGSG, GSSSG, GGGGS, GGGGSGGGGSGGGGS and the like. Those of skill in the art would be able to select the appropriate linker sequence for use in the present invention. In one embodiment, an antigen binding domain (e.g., a Tn-MUC1 binding domain, a PSMA binding domain) of the present invention comprises a heavy chain variable region (VH) and a light chain variable region (VL), wherein the VH and VL is separated by the linker sequence having the amino acid sequence GGGGSGGGGSGGGGS, which may be encoded by a nucleic acid sequence comprising the nucleotide sequence ggtggcggtggctcgggcggtggtgggtcgggt ggcggcggatct.

Despite removal of the constant regions and the introduction of a linker, scFv proteins retain the specificity of the original immunoglobulin. Single chain Fv polypeptide antibodies can be expressed from a nucleic acid comprising VH- and VL-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hybridoma (Larchmt), 27(6):455-51 (2008); Peter et al., J. Cachexia Sarcopenia Muscle (Aug. 12, 2012); Shieh et al., J. Imunol., 183(4):2277-85 (2009); Giomarelli et al., Thromb. Haemost., 97(6):955-63 (2007); Fife et al., J. Clin. Invst., 116(8):2252-61 (2006); Brocks et al., Immunotechnology, 3(3):173-84 (1997); Moosmayer et al., Ther. Immunol., 2(10):31-40 (1995). Agonistic scFvs having stimulatory activity have been described (see, e.g., Peter et al., J. Biol. Chem., 25278(38):36740-7 (2003); Xie et al., Nat. Biotech., 15(8):768-71 (1997); Ledbetter et al., Crit. Rev. Immunol., I(5-6):427-55 (1997); Ho et al., BioChim. Biophys. Acta., 1638(3):257-66 (2003)).

As used herein, “Fab” refers to a fragment of an antibody structure that binds to an antigen but is monovalent and does not have a Fc portion, for example, an antibody digested by the enzyme papain yields two Fab fragments and an Fc fragment (e.g., a heavy (H) chain constant region; Fc region that does not bind to an antigen).

In some instances, the antigen binding domain may be derived from the same species in which the CAR will ultimately be used. For example, for use in humans, the antigen binding domain of the CAR may comprise a human antibody as described elsewhere herein, or a fragment thereof.

Accordingly, an immune cell, e.g., obtained by a method described herein, can be engineered to express a CAR that target one of the following cancer associated antigens (tumor antigens): CD19; CD20; CD22 (Siglec 2); CD37; CD 123; CD22; CD30; CD 171; CS-1 (also referred to as CD2 subset 1, CRACC, SLAMF7, CD319, and 19A24); C-type lectin-like molecule-1 (CLL-1 or CLECL1); CD33; CD133; epidermal growth factor receptor (EGFR); epidermal growth factor receptor variant III (EGFRvIII); human epidermal growth factor receptor (HER1); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD 117); Interleukin-13 receptor subunit alpha-2 (IL-13Ra2 or CD213A2); Mesothelin; Interleukin 11 receptor alpha (IL-1 1Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); Folate receptor alpha; Receptor tyro sine-protein kinase ERBB2 (Her2/neu); Mucin 1, cell surface associated (MUC 1); GalNAcal-O-Ser/Thr (Tn) MUC 1 (TnMUC1); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); tyrosine-protein kinase Met (c-Met); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-la); Melanoma-associated antigen 1 (MAGE-A1); ETS translocation-variant gene 6, located on chromosome 12p (ETV6-AML); sperm protein 17 (SPA17); X Antigen Family, Member 1A (XAGEl); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B 1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B 1 (CYP1B 1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES 1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RUl); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLECI2A); bone marrow stromal cell antigen 2 (BST2); EGF-like module-containing mucin-like hormone receptor-like 2 (EMR2); lymphocyte antigen 75 (LY75); Glypican-2 (GPC2); Glypican-3 (GPC3); NKG2D; KRAS; GDNF family receptor alpha-4 (GFRa4); IL13Ra2; Fc receptor-like 5 (FCRL5); and immunoglobulin lambda-like polypeptide 1 (IGLL1). In some embodiments, the immune cell is engineered to express a CAR that targets CD19, CD20, CD22, BCMA, CD37, Mesothelin, PSMA, PSCA, Tn-MUC1, EGFR, EGFRvIII, c-Met, HER1, HER2, CD33, CD133, GD2, GPC2, GPC3, NKG2D, KRAS, or WT1.

2. Transmembrane Domain

With respect to the transmembrane domain, the CAR can be designed to comprise a transmembrane domain that connects the antigen binding domain of the CAR to the intracellular domain. The transmembrane domain of a subject CAR is a region that is capable of spanning the plasma membrane of a cell (e.g., an immune cell or precursor thereof). The transmembrane domain is for insertion into a cell membrane, e.g., a eukaryotic cell membrane. In some embodiments, the transmembrane domain is interposed between the antigen binding domain and the intracellular domain of a CAR.

In one embodiment, the transmembrane domain is naturally associated with one or more of the domains in the CAR. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein, e.g., a Type I transmembrane protein. Where the source is synthetic, the transmembrane domain may be any artificial sequence that facilitates insertion of the CAR into a cell membrane, e.g., an artificial hydrophobic sequence. Examples of the transmembrane regions of particular use in this invention include, without limitation, transmembrane domains derived from (i.e., comprise at least the transmembrane region(s) of) the alpha, beta or zeta chain of the T-cell receptor, CD28, CD2, CD3 epsilon, CD45, CD4, CD5, CD7, CD8, CD9, CD16, CD22, CD33, CD37, CD64, CD80, CD86, CD134 (OX-40), CD137 (4-1BB), CD154 (CD40L), CD278 (ICOS), CD357 (GITR), Toll-like receptor 1 (TLR1), TLR2, TLR3, TLR4, TLR5, TLR6, TLR7, TLR8, and TLR9. In some embodiments, the transmembrane domain may be synthetic, in which case it will comprise predominantly hydrophobic residues such as leucine and valine. In certain exemplary embodiments, a triplet of phenylalanine, tryptophan and valine will be found at each end of a synthetic transmembrane domain.

The transmembrane domains described herein can be combined with any of the antigen binding domains described herein, any of the costimulatory signaling domains described herein, any of the intracellular signaling domains described herein, or any of the other domains described herein that may be included in a subject CAR.

In some embodiments, the transmembrane domain further comprises a hinge region. A subject CAR of the present invention may also include a hinge region. The hinge region of the CAR is a hydrophilic region which is located between the antigen binding domain and the transmembrane domain. In some embodiments, this domain facilitates proper protein folding for the CAR. The hinge region is an optional component for the CAR. The hinge region may include a domain selected from Fc fragments of antibodies, hinge regions of antibodies, CH2 regions of antibodies, CH3 regions of antibodies, artificial hinge sequences or combinations thereof. Examples of hinge regions include, without limitation, a CD8a hinge, artificial hinges made of polypeptides which may be as small as, three glycines (Gly), as well as CH1 and CH3 domains of IgGs (such as human IgG4).

In some embodiments, a subject CAR of the present disclosure includes a hinge region that connects the antigen binding domain with the transmembrane domain, which, in turn, connects to the intracellular domain. In exemplary embodiments, the hinge region is capable of supporting the antigen binding domain to recognize and bind to the target antigen on the target cells (see, e.g., Hudecek et al., Cancer Immunol. Res., 3(2): 125-135 (2015)). In some embodiments, the hinge region is a flexible domain, thus allowing the antigen binding domain to have a structure to optimally recognize the specific structure and density of the target antigens on a cell such as tumor cell. The flexibility of the hinge region permits the hinge region to adopt many different conformations.

In some embodiments, the hinge region is an immunoglobulin heavy chain hinge region. In some embodiments, the hinge region is a hinge region polypeptide derived from a receptor (e.g., a CD8-derived hinge region).

The hinge region can have a length of from about 4 amino acids to about 50 amino acids, e.g., from about 4 amino acids to about 10 amino acids, from about 10 amino acids to about 15 amino acids, from about 15 amino acids to about 20 amino acids, from about 20 amino acids to about 25 amino acids, from about 25 amino acids to about 30 amino acids, from about 30 amino acids to about 40 amino acids, or from about 40 amino acids to about 50 amino acids.

Suitable hinge regions can be readily selected and can be of any of a number of suitable lengths, such as from about 1 amino acid (e.g., Gly) to about 20 amino acids, from about 2 amino acids to about 15 amino acids, from about 3 amino acids to about 12 amino acids, including about 4 amino acids to about 10 amino acids, about 5 amino acids to about 9 amino acids, about 6 amino acids to about 8 amino acids, or about 7 amino acids to about 8 amino acids, and can be about 1, about 2, about 3, about 4, about 5, about 6, or about 7 amino acids.

For example, hinge regions include glycine polymers (G)n, glycine-serine polymers (including, for example, (GS)n, (GSGGS)n (SEQ ID NO: 47) and (GGGS)n (SEQ ID NO: 48), where n is an integer of at least one), glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see, e.g., Scheraga, Rev. Computational. Chem. (1992) 2: 73-142). Exemplary hinge regions can comprise amino acid sequences including, but not limited to, GGSG (SEQ ID NO: 29), GGSGG (SEQ ID NO: 30), GSGSG (SEQ ID NO: 31), GSGGG (SEQ ID NO: 32), GGGSG (SEQ ID NO: 33), GSSSG (SEQ ID NO: 34), and the like.

In some embodiments, the hinge region is an immunoglobulin heavy chain hinge region. Immunoglobulin hinge region amino acid sequences are known in the art; see, e.g., Tan et al., Proc. Natd. Acad. Sci. USA, 87(1):162-166 (1990); and Huck et al., Nucleic Acids Res., 14(4): 1779-1789 (1986). As non-limiting examples, an immunoglobulin hinge region can include one of the following amino acid sequences: DKTHT (SEQ ID NO: 35); CPPC (SEQ ID NO: 36); CPEPKSCDTPPPCPR (SEQ ID NO: 37) (see, e.g., Glaser et al., J. Biol. Chem. (2005) 280:41494-41503); ELKTPLGDTTHT (SEQ ID NO: 38); KSCDKTHTCP (SEQ ID NO: 39); KCCVDCP (SEQ ID NO: 40); KYGPPCP (SEQ ID NO: 41); EPKSCDKTHTCPPCP (SEQ ID NO: 42) (human IgG1 hinge); ERKCCVECPPCP (SEQ ID NO: 43) (human IgG2 hinge); ELKTPLGDTTHTCPRCP (SEQ ID NO: 44) (human IgG3 hinge); SPNMVPHAHHAQ (SEQ ID NO: 45) (human IgG4 hinge); and the like.

The hinge region can comprise an amino acid sequence of a human IgG1, IgG2, IgG3, or IgG4, hinge region. In one embodiment, the hinge region can include one or more amino acid substitutions and/or insertions and/or deletions compared to a wild-type (naturally-occurring) hinge region. For example, His229 of human IgG1 hinge can be substituted with Tyr, so that the hinge region comprises the sequence EPKSCDKTYTCPPCP (SEQ ID NO: 46); see, e.g., Yan et al., J. Biol. Chem. (2012) 287: 5891-5897. In one embodiment, the hinge region can comprise an amino acid sequence derived from human CD8, or a variant thereof.

In one embodiment, the transmembrane domain comprises a CD8a transmembrane domain. In some embodiments, a subject CAR comprises a CD8a transmembrane domain comprising the amino acid sequence set forth in SEQ ID NO: 23, which may be encoded by a nucleic acid sequence comprising the nucleotide sequence set forth in SEQ ID NO: 24.

In another embodiment, a subject CAR comprises a CD8a hinge domain and a CD8a transmembrane domain. In one embodiment, the CD8a hinge domain comprises the amino acid sequence set forth in SEQ ID NO: 25, which may be encoded by a nucleic acid sequence comprising the nucleotide sequence set forth in SEQ ID NO: 26.

In one embodiment, the transmembrane domain comprises a CD28 transmembrane domain. In some embodiments, a subject CAR comprises a CD28 transmembrane domain comprising the amino acid sequence set forth in SEQ ID NO: 27, which may be encoded by a nucleic acid sequence comprising the nucleotide sequence set forth in SEQ ID NO: 28.

Tolerable variations of the transmembrane and/or hinge domain will be known to those of skill in the art, while maintaining its intended function. For example, in some embodiments a transmembrane domain or hinge domain comprises an amino acid sequence that has at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% sequence identity to any of the amino acid sequences set forth in SEQ ID NOs: 23, 25, and/or 27. For example, in some embodiments a transmembrane domain or hinge domain is encoded by a nucleic acid sequence comprising the nucleotide sequence that has at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% sequence identity to any of the nucleotide sequences set forth in SEQ ID NOs: 24, 26, and/or 28.

The transmembrane domain may be combined with any hinge domain and/or may comprise one or more transmembrane domains described herein.

The transmembrane domains described herein, such as a transmembrane region of alpha, beta or zeta chain of the T-cell receptor, CD28, CD2, CD3 epsilon, CD45, CD4, CD5, CD7, CD8, CD9, CD 16, CD22, CD33, CD37, CD64, CD80, CD86, CD134 (OX-40), CD137 (4-1BB), CD154 (CD40L), CD278 (ICOS), CD357 (GITR), Toll-like receptor 1 (TLR1), TLR2, TLR3, TLR4, TLR5, TLR6, TLR7, TLR8, and TLR9, can be combined with any of the antigen binding domains described herein, any of the costimulatory signaling domains or intracellular domains or cytoplasmic domains described herein, or any of the other domains described herein that may be included in the CAR.

In one embodiment, the transmembrane domain may be synthetic, in which case it will comprise predominantly hydrophobic residues such as leucine and valine. In exemplary embodiments, a triplet of phenylalanine, tryptophan and valine will be found at each end of a synthetic transmembrane domain.

In some embodiments, a subject CAR may further comprise, between the extracellular domain and the transmembrane domain of the CAR, or between the intracellular domain and the transmembrane domain of the CAR, a spacer domain. As used herein, the term “spacer domain” generally means any oligo- or polypeptide that functions to link the transmembrane domain to, either the extracellular domain or, the intracellular domain in the polypeptide chain. A spacer domain may comprise up to about 300 amino acids, e.g., about 10 to about 100 amino acids, or about 25 to about 50 amino acids. In some embodiments, the spacer domain may be a short oligo- or polypeptide linker, e.g., between about 2 and about 10 amino acids in length. For example, glycine-serine doublet provides a particularly suitable linker between the transmembrane domain and the intracellular signaling domain of the subject CAR.

Accordingly, a subject CAR of the present disclosure may comprise any of the transmembrane domains, hinge domains, or spacer domains described herein.

3. Intracellular Domain

A subject CAR of the present invention also includes an intracellular domain. The intracellular domain of the CAR is responsible for activation of at least one of the effector functions of the cell in which the CAR is expressed (e.g., immune cell). The intracellular domain transduces the effector function signal and directs the cell (e.g., immune cell) to perform its specialized function, e.g., harming and/or destroying a target cell.

The intracellular domain or otherwise the cytoplasmic domain of the CAR is responsible for activation of the cell in which the CAR is expressed. Examples of an intracellular domain for use in the invention include, but are not limited to, the cytoplasmic portion of a surface receptor, co-stimulatory molecule, and any molecule that acts in concert to initiate signal transduction in the T cell, as well as any derivative or variant of these elements and any synthetic sequence that has the same functional capability.

In certain embodiments, the intracellular domain comprises a costimulatory signaling domain. In certain embodiments, the intracellular domain comprises an intracellular signaling domain. In certain embodiments, the intracellular domain comprises a costimulatory signaling domain and an intracellular signaling domain. In certain embodiments, the intracellular domain comprises 4-1BB and CD3 zeta. In certain embodiments, the costimulatory signaling domain comprises 4-1BB. In certain embodiments, the intracellular signaling domain comprises CD3 zeta.

In one embodiment, the intracellular domain of the CAR comprises a costimulatory signaling domain which includes any portion of one or more co-stimulatory molecules, such as at least one signaling domain from proteins in the TNFR superfamily, CD27, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS (CD278), NKG2C, B7-H3 (CD276), and an intracellular domain derived from a killer immunoglobulin-like receptor (KIR, any derivative or variant thereof, any synthetic sequence thereof that has the same functional capability, and any combination thereof.

Examples of the intracellular signaling domain include, without limitation, the ζ chain of the T cell receptor complex or any of its homologs, e.g., η chain, FcsRIγ and δ chains, MB 1 (Iga) chain, B29 (Ig) chain, etc., human CD3 zeta chain, CD3 polypeptides (Δ, δ and ε), syk family tyrosine kinases (Syk, ZAP 70, etc.), src family tyrosine kinases (Lck, Fyn, Lyn, etc.), and other molecules involved in T cell transduction, such as CD2, CD5 and CD28. In one embodiment, the intracellular signaling domain may be human CD3 zeta chain, FcyRIII, FcsRI, cytoplasmic tails of Fc receptors, an immunoreceptor tyrosine-based activation motif (ITAM) bearing cytoplasmic receptors, and combinations thereof.

Other examples of the intracellular domain include a fragment or domain from one or more molecules or receptors including, but are not limited to, TCR, CD3 zeta, CD3 gamma, CD3 delta, CD3 epsilon, CD86, common FcR gamma, FcR beta (Fc Epsilon Rib), CD79a, CD79b, Fc gamma RI 1a, DAP10, DAP12, T cell receptor (TCR), CD8, CD27, CD28, 4-1BB (CD137), OX9, OX40, CD30, CD40, PD-1, ICOS, a KIR family protein, lymphocyte function-associated antigen-1 (LFA-1), CD2, CD7, LIGHT, NKG2C, B7-H3, a ligand that specifically binds with CD83, CD5, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD1Id, ITGAE, CD103, ITGAL, CD11a, LFA-1, ITGAM, CD lib, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAMI, CRTAM, Ly9 (CD229), CD160 (BY55), PSGL1, CD100 (SEMA4D), CD69, SLAMF6 (NTB-A, Lyl08), SLAM (SLAMFI, CD150, IPO-3), BLAME (SLAMF8), SELPLG (CD 162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co-stimulatory molecule that has the same functional capability, and any combination thereof.

Additional examples of intracellular domains include, without limitation, intracellular signaling domains of several types of various other immune signaling receptors, including, but not limited to, first, second, and third generation T cell signaling proteins including CD3, B7 family costimulatory, and Tumor Necrosis Factor Receptor (TNFR) superfamily receptors (see, e.g., Park and Brentjens, J. Clin. Oncol., 33(6): 651-653 (2015)). Additionally, intracellular signaling domains may include signaling domains used by NK and NKT cells (see, e.g., Hermanson and Kaufman, Front. Immunol., 6: 195 (2015)) such as signaling domains of NKp30 (B7-H6) (see, e.g., Zhang et al., J. Immunol., 189(5): 2290-2299 (2012)), and DAP 12 (see, e.g., Topfer et al., J. Immunol., 194(7): 3201-3212 (2015)), NKG2D, NKp44, NKp46, DAP10, and CD3z.

Intracellular signaling domains suitable for use in a subject CAR of the present invention include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation of the CAR (i.e., activated by antigen and dimerizing agent). In some embodiments, the intracellular signaling domain includes at least one (e.g., one, two, three, four, five, six, etc.) ITAM motifs as described below. In some embodiments, the intracellular signaling domain includes DAP10/CD28 type signaling chains. In some embodiments, the intracellular signaling domain is not covalently attached to the membrane bound CAR, but is instead diffused in the cytoplasm.

Intracellular signaling domains suitable for use in a subject CAR of the present invention include immunoreceptor tyrosine-based activation motif (ITAM)-containing intracellular signaling polypeptides. In some embodiments, an ITAM motif is repeated twice in an intracellular signaling domain, where the first and second instances of the ITAM motif are separated from one another by 6 to 8 amino acids. In one embodiment, the intracellular signaling domain of a subject CAR comprises 3 ITAM motifs. In some embodiments, intracellular signaling domains includes the signaling domains of human immunoglobulin receptors that contain immunoreceptor tyrosine based activation motifs (ITAMs) such as, but not limited to, Fc gamma RI, Fc gamma RIIA, Fc gamma RIIC, Fc gamma RIIIA, FcRL5 (see, e.g., Gillis et al., Front. Immunol., 5:254 (2014)).

A suitable intracellular signaling domain can be an ITAM motif-containing portion that is derived from a polypeptide that contains an ITAM motif. For example, a suitable intracellular signaling domain can be an ITAM motif-containing domain from any ITAM motif-containing protein. Thus, a suitable intracellular signaling domain need not contain the entire sequence of the entire protein from which it is derived. Examples of suitable ITAM motif-containing polypeptides include, but are not limited to: DAP12, FCER1G (Fc epsilon receptor I gamma chain), CD3D (CD3 delta), CD3E (CD3 epsilon), CD3G (CD3 gamma), CD3Z (CD3 zeta), and CD79A (antigen receptor complex-associated protein alpha chain).

In one embodiment, the intracellular signaling domain is derived from DAP12 (also known as TYROBP; TYRO protein tyrosine kinase binding protein; KARAP; PLOSL; DNAX-activation protein 12; KAR-associated protein; TYRO protein tyrosine kinase-binding protein; killer activating receptor associated protein; killer-activating receptor-associated protein; etc.). In one embodiment, the intracellular signaling domain is derived from FCER1G (also known as FCRG; Fc epsilon receptor I gamma chain; Fc receptor gamma-chain; fc-epsilon RI-gamma; fcR gamma; fceR1 gamma; high affinity immunoglobulin epsilon receptor subunit gamma; immunoglobulin E receptor, high affinity, gamma chain; etc.). In one embodiment, the intracellular signaling domain is derived from T-cell surface glycoprotein CD3 delta chain (also known as CD3D; CD3-DELTA; T3D; CD3 antigen, delta subunit; CD3 delta; CD3d antigen, delta polypeptide (TiT3 complex); OKT3, delta chain; T-cell receptor T3 delta chain; T-cell surface glycoprotein CD3 delta chain; etc.). In one embodiment, the intracellular signaling domain is derived from T-cell surface glycoprotein CD3 epsilon chain (also known as CD3e, T-cell surface antigen T3/Leu-4 epsilon chain, T-cell surface glycoprotein CD3 epsilon chain, AI504783, CD3, CD3epsilon, T3e, etc.). In one embodiment, the intracellular signaling domain is derived from T-cell surface glycoprotein CD3 gamma chain (also known as CD3G, T-cell receptor T3 gamma chain, CD3-GAMMA, T3G, gamma polypeptide (TiT3 complex), etc.). In one embodiment, the intracellular signaling domain is derived from T-cell surface glycoprotein CD3 zeta chain (also known as CD3Z, T-cell receptor T3 zeta chain, CD247, CD3-ZETA, CD3H, CD3Q, T3Z, TCRZ, etc.). In one embodiment, the intracellular signaling domain is derived from CD79A (also known as B-cell antigen receptor complex-associated protein alpha chain; CD79a antigen (immunoglobulin-associated alpha); MB-1 membrane glycoprotein; Ig-alpha; membrane-bound immunoglobulin-associated protein; surface IgM-associated protein; etc.). In one embodiment, an intracellular signaling domain suitable for use in a subject CAR of the present disclosure includes a DAP10/CD28 type signaling chain. In one embodiment, an intracellular signaling domain suitable for use in a subject CAR of the present disclosure includes a ZAP70 polypeptide. In some embodiments, the intracellular signaling domain includes a cytoplasmic signaling domain of TCR zeta, FcR gamma, FcR beta, CD3 gamma, CD3 delta, CD3 epsilon, CD5, CD22, CD79a, CD79b, or CD66d. In one embodiment, the intracellular signaling domain in the CAR includes a cytoplasmic signaling domain of human CD3 zeta.

While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The intracellular signaling domain includes any truncated portion of the intracellular signaling domain sufficient to transduce the effector function signal.

The intracellular signaling domains described herein can be combined with any of the costimulatory signaling domains described herein, any of the antigen binding domains described herein, any of the transmembrane domains described herein, or any of the other domains described herein that may be included in the CAR.

Further, variant intracellular signaling domains suitable for use in a subject CAR are known in the art. The YMFM motif is found in ICOS and is a SH2 binding motif that recruits both p85 and p50alpha subunits of PI3K, resulting in enhanced AKT signaling. See, e.g., Simpson et al. (2010) Curr. Opin. Immunol., 22:326-332. In one embodiment, a CD28 intracellular domain variant may be generated to comprise a YMFM motif.

In one embodiment, the intracellular domain of a subject CAR comprises a 4-1BB costimulatory domain comprising the amino acid sequence set forth in SEQ ID NO: 1, which may be encoded by a nucleic acid sequence comprising the nucleotide sequence set forth in SEQ ID NO: 2 or 3. In one embodiment, the intracellular domain of a subject CAR comprises a CD28 costimulatory domain comprising the amino acid sequence set forth in SEQ ID NO: 4, which may be encoded by a nucleic acid sequence comprising the nucleotide sequence set forth in SEQ ID NO: 5. In one embodiment, the intracellular domain of a subject CAR comprises a CD28(YMFM) costimulatory domain comprising the amino acid sequence set forth in SEQ ID NO: 6, which may be encoded by a nucleic acid sequence comprising the nucleotide sequence set forth in SEQ ID NO: 7. In one embodiment, the intracellular domain of a subject CAR comprises an ICOS costimulatory domain comprising the amino acid sequence set forth in SEQ ID NO: 8, which may be encoded by a nucleic acid sequence comprising the nucleotide sequence set forth in SEQ ID NO: 9 or SEQ ID NO: 10. In one embodiment, the intracellular domain of a subject CAR comprises an ICOS(YMNM) costimulatory domain comprising the amino acid sequence set forth in SEQ ID NO: 11, which may be encoded by a nucleic acid sequence comprising the nucleotide sequence set forth in SEQ ID NO: 12. In one embodiment, the intracellular domain of a subject CAR comprises a CD2 costimulatory domain comprising the amino acid sequence set forth in SEQ ID NO: 13, which may be encoded by a nucleic acid sequence comprising the nucleotide sequence set forth in SEQ ID NO: 14. In one embodiment, the intracellular domain of a subject CAR comprises a CD27 costimulatory domain comprising the amino acid sequence set forth in SEQ ID NO: 15, which may be encoded by a nucleic acid sequence comprising the nucleotide sequence set forth in SEQ ID NO: 16. In one embodiment, the intracellular domain of a subject CAR comprises a OX40 costimulatory domain comprising the amino acid sequence set forth in SEQ ID NO: 17, which may be encoded by a nucleic acid sequence comprising the nucleotide sequence set forth in SEQ ID NO: 18.

In one embodiment, the intracellular domain of a subject CAR comprises a CD3 zeta intracellular signaling domain comprising the amino acid sequence set forth in SEQ ID NO: 19 or SEQ ID NO: 21, which may be encoded by a nucleic acid sequence comprising the nucleotide sequence set forth in SEQ ID NO: 20 or SEQ ID NO: 22, respectively.

Tolerable variations of the intracellular domain will be known to those of skill in the art, while maintaining specific activity. For example, in some embodiments the intracellular domain comprises an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any of the amino acid sequences set forth in SEQ ID NO: 19 or 21. For example, in some embodiments the intracellular domain is encoded by a nucleic acid sequence comprising a nucleotide sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any of the nucleotide sequences set forth in SEQ ID NO: 20 or 22.

In one embodiment, the intracellular domain of a subject CAR comprises an ICOS costimulatory domain and a CD3 zeta intracellular signaling domain. In one embodiment, the intracellular domain of a subject CAR comprises a CD28 costimulatory domain and a CD3 zeta intracellular signaling domain. In one embodiment, the intracellular domain of a subject CAR comprises a CD28 YMFM variant costimulatory domain and a CD3 zeta intracellular signaling domain. In one embodiment, the intracellular domain of a subject CAR comprises a CD27 costimulatory domain and a CD3 zeta intracellular signaling domain. In one embodiment, the intracellular domain of a subject CAR comprises a OX40 costimulatory domain and a CD3 zeta intracellular signaling domain. In one exemplary embodiment, the intracellular domain of a subject CAR comprises a 4-1BB costimulatory domain and a CD3 zeta intracellular signaling domain. In one exemplary embodiment, the intracellular domain of a subject CAR comprises a CD2 costimulatory domain and a CD3 zeta intracellular signaling domain.

Table 1 illustrates exemplary sequences of the domains of a CAR described herein.

TABLE 1

| SEQ ID | | |
|---|---|---|
| NO: | Description | Sequence |
| 1 | 4-1BB costimulatory | KRGRKKLLYIFKQPFMRPVQTTQEEDGCSCRFPEEEEGG |
| | domain amino acid | CEL |
| | sequence | |
| 2 | 4-1BB costimulatory | AAACGGGGCAGAAAGAAACTCCTGTATATATTCAAAC |
| | domain nucleic acid | AACCATTTATGAGACCAGTACAAACTACTCAAGAGGA |
| | sequence #1 | AGACGGCTGTAGCTGCCGATTTCCAGAAGAAGAAGAA |
| | | GGAGGATGTGAACTG |
| 3 | 4-1BB costimulatory | AAACGGGGCAGAAAGAAACTCCTGTATATATTCAAAC |
| | domain nucleic acid | AACCATTTATGAGACCAGTACAAACTACTCAAGAGGA |
| | sequence #2 | AGATGGCTGTAGCTGCCGATTTCCAGAAGAAGAAGAA |
| | | GGAGGATGTGAACTG |
| 4 | CD28 costimulatory | RSKRSRLLHSDYMNMTPRRPGPTRKHYQPYAPPRDFAA |
| | domain amino acid | YRS |
| | sequence | |
| 5 | CD28 costimulatory | AGGAGTAAGAGGAGCAGGCTCCTGCACAGTGACTACA |
| | domain nucleic acid | TGAACATGACTCCCCGCCGCCCCGGGCCCACCCGCAA |
| | sequence | GCATTACCAGCCCTATGCCCCACCACGCGACTTCGCAG |
| | | CCTATCGCTCC |
| 6 | CD28(YMFM) | RSKRSRLLHSDYMFMTPRRPGPTRKHYQPYAPPRDFAAY |
| | costimulatory domain | RS |
| | amino acid sequence | |
| 7 | CD28(YMFM) | AGGAGTAAGAGGAGCAGGCTCCTGCACAGTGACTACA |
| | costimulatory domain | TGTTCATGACTCCCCGCCGCCCCGGGCCCACCCGCAAG |
| | nucleic acid sequence | CATTACCAGCCCTATGCCCCACCACGCGACTTCGCAGC |
| | | CTATCGCTCC |
| 8 | ICOS costimulatory | TKKKYSSSVHDPNGEYMFMRAVNTAKKSRLTDVTL |
| | domain amino acid | |
| | sequence | |
| 9 | ICOS costimulatory | ACAAAAAAGAAGTATTCATCCAGTGTGCACGACCCTA |
| | domain nucleic acid | ACGGTGAATACATGTTCATGAGAGCAGTGAACACAGC |
| | sequence #1 | CAAAAAATCCAGACTCACAGATGTGACCCTA |
| 10 | ICOS costimulatory | ACAAAAAAGAAGTATTCATCCAGTGTGCACGACCCTA |
| | domain nucleic acid | ACGGTGAATACATGTTCATGAGAGCAGTGAACACAGC |
| | sequence #2 | CAAAAAATCTAGACTCACAGATGTGACCCTA |
| 11 | ICOS(YMNM) | TKKKYSSSVHDPNGEYMNMRAVNTAKKSRLTDVTL |
| | costimulatory domain | |
| | amino acid sequence | |
| 12 | ICOS(YMNM) | ACAAAAAAGAAGTATTCATCCAGTGTGCACGACCCTA |
| | costimulatory domain | ACGGTGAATACATGAACATGAGAGCAGTGAACACAGC |
| | nucleic acid sequence | CAAAAAATCCAGACTCACAGATGTGACCCTA |
| 13 | CD2 costimulatory | TKRKKQRSRRNDEELETRAHRVATEERGRKPHQIPASTP |
| | domain amino acid | QNPATSQHPPPPPGHRSQAPSHRPPPPGHRVQHQPQKRPP |
| | sequence | APSGTQVHQQKGPPLPRPRVQPKPPHGAAENSLSPSSN |
| 14 | CD2 costimulatory | ACCAAAAGGAAAAAACAGAGGAGTCGGAGAAATGAT |
| | domain nucleic acid | GAGGAGCTGGAGACAAGAGCCCACAGAGTAGCTACTG |
| | sequence | AAGAAAGGGGCCGGAAGCCCCACCAAATTCCAGCTTC |
| | | AACCCCTCAGAATCCAGCAACTTCCCAACATCCTCCTC |
| | | CACCACCTGGTCATCGTTCCCAGGCACCTAGTCATCGT |
| | | CCCCCGCCTCCTGGACACCGTGTTCAGCACCAGCCTCA |
| | | GAAGAGGCCTCCTGCTCCGTCGGGCACACAAGTTCAC |
| | | CAGCAGAAAGGCCCGCCCCTCCCCAGACCTCGAGTTC |
| | | AGCCAAAACCTCCCCATGGGGCAGCAGAAAACTCATT |
| | | GTCCCCTTCCTCTAAT |
| 15 | CD27 costimulatory | QRRKYRSNKGESPVEPAEPCRYSCPREEEGSTIPIQEDYR |
| | domain amino acid | KPEPACSP |
| | sequence | |
| 16 | CD27 costimulatory | CAACGAAGGAAATATAGATCAAACAAAGGAGAAAGT |
| | domain nucleic acid | CCTGTGGAGCCTGCAGAGCCTTGTCGTTACAGCTGCCC |
| | sequence | CAGGGAGGAGGAGGGCAGCACCATCCCCATCCAGGAG |
| | | GATTACCGAAAACCGGAGCCTGCCTGCTCCCCC |
| 17 | OX40 costimulatory | ALYLLRRDQRLPPDAHKPPGGGSFRTPIQEEQADAHSTLA |
| | domain amino acid | KI |
| | sequence | |
| 18 | OX40 costimulatory | GCCCTGTACCTGCTCCGCAGGGACCAGAGGCTGCCCCC |
| | domain nucleic acid | CGATGCCCACAAGCCCCCTGGGGGAGGCAGTTTCAGG |
| | sequence | ACCCCCATCCAAGAGGAGCAGGCCGACGCCCACTCCA |
| | | CCCTGGCCAAGATC |
| 19 | CD3 zeta intracellular | RVKFSRSADAPAYQQGQNQLYNELNLGRREEYDVLDKR |
| | signaling domain | RGRDPEMGGKPRRKNPQEGLYNELQKDKMAEAYSEIGM |
| | amino acid sequence | KGERRRGKGHDGLYQGLSTATKDTYDALHMQALPPR |
| 20 | CD3 zeta intracellular | AGAGTGAAGTTCAGCAGGAGCGCAGACGCCCCCGCGT |
| | signaling domain | ACCAGCAGGGCCAGAACCAGCTCTATAACGAGCTCAA |
| | nucleic acid sequence | TCTAGGACGAAGAGAGGAGTACGATGTTTTGGACAAG |
| | | AGACGTGGCCGGGACCCTGAGATGGGGGGAAAGCCGA |
| | | GAAGGAAGAACCCTCAGGAAGGCCTGTACAATGAACT |
| | | GCAGAAAGATAAGATGGCGGAGGCCTACAGTGAGATT |
| | | GGGATGAAAGGCGAGCGCCGGAGGGGCAAGGGGCAC |
| | | GATGGCCTTTACCAGGGTCTCAGTACAGCCACCAAGG |
| | | ACACCTACGACGCCCTTCACATGCAGGCCCTGCCCCCT |
| | | CGC |
| 21 | CD3 zeta (Q14K) | RVKFSRSADAPAYKQGQNQLYNELNLGRREEYDVLDKR |
| | intracellular signaling | RGRDPEMGGKPRRKNPQEGLYNELQKDKMAEAYSEIGM |
| | domain amino acid | KGERRRGKGHDGLYQGLSTATKDTYDALHMQALPPR |
| | sequence | |
| 22 | CD3 zeta (Q14K) | AGAGTGAAGTTCAGCAGGAGCGCAGACGCCCCCGCGT |
| | intracellular signaling | ACAAGCAGGGCCAGAACCAGCTCTATAACGAGCTCAA |
| | domain nucleic acid | TCTAGGACGAAGAGAGGAGTACGATGTTTTGGACAAG |
| | sequence | AGACGTGGCCGGGACCCTGAGATGGGGGGAAAGCCGA |
| | | GAAGGAAGAACCCTCAGGAAGGCCTGTACAATGAACT |
| | | GCAGAAAGATAAGATGGCGGAGGCCTACAGTGAGATT |
| | | GGGATGAAAGGCGAGCGCCGGAGGGGCAAGGGGCAC |
| | | GATGGCCTTTACCAGGGTCTCAGTACAGCCACCAAGG |
| | | ACACCTACGACGCCCTTCACATGCAGGCCCTGCCCCCT |
| | | CGC |
| 23 | CD8 alpha | IYIWAPLAGTCGVLLLSLVITLYC |
| | transmembrane domain | |
| | amino acid sequence | |
| 24 | CD8 alpha | ATCTACATCTGGGCGCCCTTGGCCGGGACTTGTGGGGT |
| | transmembrane domain | CCTTCTCCTGTCACTGGTTATCACCCTTTACTGC |
| | nucleic acid sequence | |
| 25 | CD8 alpha hinge | TTTPAPRPPTPAPTIASQPLSLRPEACRPAAGGAVHTRGL |
| | domain amino acid | DFACD |
| | sequence | |
| 26 | CD8 alpha hinge | ACCACGACGCCAGCGCCGCGACCACCAACACCGGCGC |
| | domain nucleic acid | CCACCATCGCGTCGCAGCCCCTGTCCCTGCGCCCAGAG |
| | sequence | GCGTGCCGGCCAGCGGGGGGGGCGCAGTGCACACGA |
| | | GGGGGCTGGACTTCGCCTGTGAT |
| 27 | CD28 transmembrane | FWVLVVVGGVLACYSLLVTVAFIIFWV |
| | domain amino acid | |
| | sequence | |
| 28 | CD28 transmembrane | TTTTGGGTGCTGGTGGTGGTTGGTGGAGTCCTGGCTTG |
| | domain nucleic acid | CTATAGCTTGCTAGTAACAGTGGCCTTTATTATTTTCTG |
| | sequence | GGTG |

### B. T Cell Receptors

In some embodiments, the modified unstimulated immune cell or modified stimulated immune cell (e.g., modified unstimulated or stimulated T cell) expresses an exogenous T cell receptor (TCR). Any modified cell comprising a TCR comprising affinity for any antigen (e.g., solid tumor antigen) is envisioned, and can readily be understood and made by a person of skill in the art in view of the disclosure herein.

In some embodiments, the target cell has been altered to contain specific T cell receptor (TCR) genes (e.g., a TRAC and TRBC gene). TCRs or antigen-binding portions thereof include those that recognize a peptide epitope or T cell epitope of a target polypeptide, such as an antigen of a tumor, viral or autoimmune protein. In some embodiments, the TCR has binding specificity for a tumor associated antigen. In some instances, exemplary tumor associated antigens include NY-ESO-1 (LAGE2, LAGE2B3, or cancer/testis antigen 1), melanoma-associated antigen (MAGE), and H3.3K27M.

A TCR is a disulfide-linked heterodimeric protein comprised of six different membrane bound chains that participate in the activation of T cells in response to an antigen. There exists alpha/beta TCRs and gamma/delta TCRs. An alpha/beta TCR comprises a TCR alpha chain and a TCR beta chain. T cells expressing a TCR comprising a TCR alpha chain and a TCR beta chain are commonly referred to as alpha/beta T cells. Gamma/delta TCRs comprise a TCR gamma chain and a TCR delta chain. T cells expressing a TCR comprising a TCR gamma chain and a TCR delta chain are commonly referred to as gamma/delta T cells. A TCR of the present disclosure is a TCR comprising a TCR alpha chain and a TCR beta chain. The TCR alpha chain and the TCR beta chain are each comprised of two extracellular domains, a variable region and a constant region. The TCR alpha chain variable region and the TCR beta chain variable region are required for the affinity of a TCR to a target antigen. Each variable region comprises three hypervariable or complementarity determining regions (CDRs) which provide for binding to a target antigen. The constant region of the TCR alpha chain and the constant region of the TCR beta chain are proximal to the cell membrane. A TCR further comprises a transmembrane region and a short cytoplasmic tail. CD3 molecules are assembled together with the TCR heterodimer. CD3 molecules comprise a characteristic sequence motif for tyrosine phosphorylation, known as immunoreceptor tyrosine-based activation motifs (ITAMs). Proximal signaling events are mediated through the CD3 molecules, and accordingly, TCR-CD3 complex interaction plays an important role in mediating cell recognition events.

Stimulation of TCR is triggered by major histocompatibility complex molecules (MICs) on antigen presenting cells that present antigen peptides to T cells and interact with TCRs to induce a series of intracellular signaling cascades. Engagement of the TCR initiates both positive and negative signaling cascades that result in cellular proliferation, cytokine production, and/or activation-induced cell death.

A TCR of the present invention can be a wild-type TCR, a high affinity TCR, and/or a chimeric TCR. A high affinity TCR may be the result of modifications to a wild-type TCR that confers a higher affinity for a target antigen compared to the wild-type TCR. A high affinity TCR may be an affinity-matured TCR. Methods for modifying TCRs and/or the affinity-maturation of TCRs are known to those of skill in the art. Techniques for engineering and expressing TCRs include, but are not limited to, the production of TCR heterodimers which include the native disulphide bridge which connects the respective subunits (Garboczi, et al., (1996), Nature 384(6605): 134-41; Garboczi, et al., (1996), J Immunol 157(12): 5403-10; Chang et al., (1994), PNAS USA 91: 1 1408-1 1412; Davodeau et al., (1993), J. Biol. Chem. 268(21): 15455-15460; Golden et al., (1997), J. Imm. Meth. 206: 163-169; U.S. Pat. No. 6,080,840).

In some embodiments, the exogenous TCR is a full TCR or antigen-binding portions or antigen-binding fragments thereof. In some embodiments, the TCR is an intact or full-length TCR, including TCRs in the ab form or gd form. In some embodiments, the TCR is an antigen binding portion that is less than a full-length TCR but that binds to a specific peptide bound in an MHC molecule, such as binds to an MHC-peptide complex. In some cases, an antigen binding portion or fragment of a TCR can contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable a chain and variable b chain of a TCR, sufficient to form a binding site for binding to a specific MHC-peptide complex. Generally, the variable chains of a TCR contain complementarity determining regions (CDRs) involved in recognition of the peptide, MHC and/or MHC-peptide complex.

In some embodiments, the variable domains of the TCR contain hypervariable loops, or CDRs, which generally are the primary contributors to antigen recognition and binding capabilities and specificity. In some embodiments, a CDR of a TCR or combination thereof forms all or substantially all of the antigen-binding site of a given TCR molecule. The various CDRs within a variable region of a TCR chain generally are separated by framework regions (FRs), which generally display less variability among TCR molecules as compared to the CDRs (see, e.g., Jores et al, PNAS, 87:9138 (1990); Chothia et al., EMBO J., 7:3745 (1988); see also Lefranc et al., Dev. Comp. Immunol., 27:55 (2003)). In some embodiments, CDR3 is the main CDR responsible for antigen binding or specificity, or is the most important among the three CDRs on a given TCR variable region for antigen recognition, and/or for interaction with the processed peptide portion of the peptide-MHC complex. In some contexts, the CDR1 of the alpha chain can interact with the N-terminal part of certain antigenic peptides. In some contexts, CDR1 of the beta chain can interact with the C-terminal part of the peptide. In some contexts, CDR2 contributes most strongly to or is the primary CDR responsible for the interaction with or recognition of the MHC portion of the MHC-peptide complex. In some embodiments, the variable region of the b-chain can contain a further hypervariable region (CDR4 or HVR4), which generally is involved in superantigen binding and not antigen recognition (Kotb, Clinical Microbiology Reviews, 8:41 1-426 (1995)).

In some embodiments, a TCR contains a variable alpha domain (Va) and/or a variable beta domain (Vp) or antigen-binding fragments thereof. In some embodiments, the α-chain and/or β-chain of a TCR also can contain a constant domain, a transmembrane domain and/or a short cytoplasmic tail (see, e.g., Janeway et al., Immunobiology: The Immune System in Health and Disease, 3 Ed., Current Biology Publications, p. 4:33 (1997)).

In some embodiments, the a chain constant domain is encoded by the TRAC gene (IMGT nomenclature) or is a variant thereof. In some embodiments, the R chain constant region is encoded by TRBC1 or TRBC2 genes (IMGT nomenclature) or is a variant thereof. In some embodiments, the constant domain is adjacent to the cell membrane. For example, in some cases, the extracellular portion of the TCR formed by the two chains contains two membrane-proximal constant domains, and two membrane-distal variable domains, which variable domains each contain CDRs.

It is within the level of a skilled artisan to determine or identify the various domains or regions of a TCR. In some aspects, residues of a TCR are known or can be identified according to the International Immunogenetics Information System (IMGT) numbering system (see e.g. www.imgt.org; see also, Lefranc et al., Developmental and Comparative Immunology, 2:55-77 (2003); and The T Cell Factsbook 2nd Edition, Lefranc and LeFranc Academic Press 2001). Using this system, the CDR1 sequences within a TCR Va chain and/or nb chain correspond to the amino acids present between residue numbers 27-38, inclusive, the CDR2 sequences within a TCR Va chain and/or nb chain correspond to the amino acids present between residue numbers 56-65, inclusive, and the CDR3 sequences within a TCR Va chain and/or nb chain correspond to the amino acids present between residue numbers 105-117, inclusive.

In some embodiments, the TCR may be a heterodimer of two chains a and b (or optionally g and d) that are linked, such as by a disulfide bond or disulfide bonds. In some embodiments, the constant domain of the TCR may contain short connecting sequences in which a cysteine residue forms a disulfide bond, thereby linking the two chains of the TCR. In some embodiments, a TCR may have an additional cysteine residue in each of the a and b chains, such that the TCR contains two disulfide bonds in the constant domains. In some embodiments, each of the constant and variable domains contain disulfide bonds formed by cysteine residues.

In some embodiments, the TCR for engineering cells as described is one generated from a known TCR sequence(s), such as sequences of na,b chains, for which a substantially full-length coding sequence is readily available. Methods for obtaining full-length TCR sequences, including V chain sequences, from cell sources are well known. In some embodiments, nucleic acids encoding the TCR can be obtained from a variety of sources, such as by polymerase chain reaction (PCR) amplification of TCR-encoding nucleic acids within or isolated from a given cell or cells, or synthesis of publicly available TCR DNA sequences. In some embodiments, the TCR is obtained from a biological source, such as from cells such as from a T cell (e.g. cytotoxic T cell), T-cell hybridomas or other publicly available source. In some embodiments, the T-cells can be obtained from in vivo isolated cells. In some embodiments, the T-cells can be a cultured T-cell hybridoma or clone. In some embodiments, the TCR or antigen-binding portion thereof can be synthetically generated from knowledge of the sequence of the TCR. In some embodiments, a high-affinity T cell clone for a target antigen (e.g., a cancer antigen) is identified, isolated from a patient, and introduced into the cells. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al., Clin Cancer Res., 15: 169-180 (2009) and Cohen et al., J. Immunol., 175:5799-5808 (2005). In some embodiments, phage display is used to isolate TCRs against a target antigen (see, e.g., Varela-Rohena et al., Nat. Med., 14: 1390-1395 (2008) and Li, Nat. Biotechnol., 23:349-354 (2005). In some embodiments, the TCR or antigen-binding portion thereof is one that has been modified or engineered. In some embodiments, directed evolution methods are used to generate TCRs with altered properties, such as with higher affinity for a specific MHC-peptide complex. In some embodiments, directed evolution is achieved by display methods including, but not limited to, yeast display (Holler et al., Nat. Immunol., 4:55-62 (2003); Holler et al., PNAS USA, 97: 5387-92 (2000)), phage display (Li et al., Nat. Biotechnol., 23: 349-54 (2005)), or T cell display (Chervin et al., J. Immunol. Methods, 339:175-84 (2008)). In some embodiments, display approaches involve engineering, or modifying, a known, parent or reference TCR. For example, in some cases, a wild-type TCR can be used as a template for producing mutagenized TCRs in which in one or more residues of the CDRs are mutated, and mutants with an desired altered property, such as higher affinity for a desired target antigen, are selected.

In some embodiments as described, the TCR can contain an introduced disulfide bond or bonds. In some embodiments, the native disulfide bonds are not present. In some embodiments, the one or more of the native cysteines (e.g. in the constant domain of the a chain and b chain) that form a native interchain disulfide bond are substituted to another residue, such as to a serine or alanine. In some embodiments, an introduced disulfide bond can be formed by mutating non-cysteine residues on the alpha and beta chains, such as in the constant domain of the a chain and b chain, to cysteine. Exemplary non-native disulfide bonds of a TCR are described in WO 2006/000830 and WO 2006/037960. In some embodiments, cysteines can be introduced at residue Thr48 of the alpha chain and Ser57 of the beta chain, at residue Thr45 of the alpha chain and Ser77 of the beta chain, at residue TyrIO of the alpha chain and Serl7 of the beta chain, at residue Thr45 of the alpha chain and Asp59 of the beta chain and/or at residue Serl5 of the alpha chain and Glul5 of the beta chain. In some embodiments, the presence of non-native cysteine residues (e.g. resulting in one or more non-native disulfide bonds) in a recombinant TCR can favor production of the desired recombinant TCR in a cell in which it is introduced over expression of a mismatched TCR pair containing a native TCR chain.

In some embodiments, the TCR chains contain a transmembrane domain. In some embodiments, the transmembrane domain is positively charged. In some cases, the TCR chain contains a cytoplasmic tail. In some aspects, each chain (e.g. alpha or beta) of the TCR can possess one N-terminal immunoglobulin variable domain, one immunoglobulin constant domain, a transmembrane region, and a short cytoplasmic tail at the C-terminal end. In some embodiments, a TCR, for example via the cytoplasmic tail, is associated with invariant proteins of the CD3 complex involved in mediating signal transduction. In some cases, the structure allows the TCR to associate with other molecules like CD3 and subunits thereof. For example, a TCR containing constant domains with a transmembrane region may anchor the protein in the cell membrane and associate with invariant subunits of the CD3 signaling apparatus or complex. The intracellular tails of CD3 signaling subunits (e.g. CD3Y, CD36, CD3s and O\ddot{ν}3z chains) contain one or more immunoreceptor tyrosine-based activation motifs or ITAMs that are involved in the signaling capacity of the TCR complex.

In some embodiments, the TCR is a full-length TCR. In some embodiments, the TCR is an antigen-binding portion. In some embodiments, the TCR is a dimeric TCR (dTCR). In some embodiments, the TCR is a single-chain TCR (sc-TCR). A TCR may be cell-bound or in soluble form. In some embodiments, for purposes of the provided methods, the TCR is in cell-bound form expressed on the surface of a cell. In some embodiments a dTCR contains a first polypeptide wherein a sequence corresponding to a TCR a chain variable region sequence is fused to the N terminus of a sequence corresponding to a TCR a chain constant region extracellular sequence, and a second polypeptide wherein a sequence corresponding to a TCR b chain variable region sequence is fused to the N terminus a sequence corresponding to a TCR b chain constant region extracellular sequence, the first and second polypeptides being linked by a disulfide bond. In some embodiments, the bond can correspond to the native interchain disulfide bond present in native dimeric ab TCRs. In some embodiments, the interchain disulfide bonds are not present in a native TCR. For example, in some embodiments, one or more cysteines can be incorporated into the constant region extracellular sequences of dTCR polypeptide pair. In some cases, both a native and a non-native disulfide bond may be desirable. In some embodiments, the TCR contains a transmembrane sequence to anchor to the membrane. In some embodiments, a dTCR contains a TCR a chain containing a variable a domain, a constant a domain and a first dimerization motif attached to the C-terminus of the constant a domain, and a TCR b chain comprising a variable b domain, a constant b domain and a first dimerization motif attached to the C-terminus of the constant b domain, wherein the first and second dimerization motifs easily interact to form a covalent bond between an amino acid in the first dimerization motif and an amino acid in the second dimerization motif linking the TCR a chain and TCR b chain together.

In some embodiments, the TCR is a scTCR, which is a single amino acid strand containing an a chain and a p chain that is able to bind to MHC-peptide complexes.

Typically, a scTCR can be generated using methods known to those of skill in the art, See e.g., WO 1996/13593, WO 1996/18105, WO 1999/18129, WO 2004/033685, W) 2006/037960, WO 2011/044186; U.S. Pat. No. 7,569,664; and Schlueter et al. J. Mol. Biol., 256:859 (1996).

In some embodiments, the transmembrane domain can be a Ca or CP transmembrane domain. In some embodiments, the transmembrane domain can be from a non-TCR origin, for example, a transmembrane region from CD3z, CD28 or B7.1. In some embodiments, the TCR does contain a sequence corresponding to cytoplasmic sequences. In some embodiments, the TCR contains a CD3z signaling domain. In some embodiments, the TCR is capable of forming a TCR complex with CD3. In some embodiments, the TCR or antigen binding portion thereof may be a recombinantly produced natural protein or mutated form thereof in which one or more property, such as binding characteristic, has been altered. In some embodiments, a TCR may be derived from one of various animal species, such as human, mouse, rat, or other mammal.

In some embodiments, the TCR comprises affinity to a target antigen on a target cell. The target antigen may include any type of protein, or epitope thereof, associated with the target cell. For example, the TCR may comprise affinity to a target antigen on a target cell that indicates a particular disease state of the target cell. In some embodiments, the target antigen is processed and presented by MHCs.

In some embodiments, the TCR is encoded by a nucleic acid construct that encodes sequentially, from N-terminus to C-terminus a first heterologous TCR subunit chain, wherein the TCR subunit chain comprises the variable region and the constant region of the TCR subunit chain, and a variable region and a variable region of a second heterologous TCR subunit chain. The construct further encodes a first self-cleaving peptide that precedes the variable region of the first heterologous TCR subunit chain, and a second self-cleaving peptide between the first heterologous TCR subunit chain and the second TCR subunit. In some embodiments, the first heterologous TCR subunit chain is a TCRα chain, and the second heterologous TCR subunit chain is a TCRβ chain. In some embodiments, the first heterologous TCR subunit chain is a TCRβ chain, and the second heterologous TCR subunit chain is a TCRα chain.

Examples of self-cleaving peptides include, but are not limited to, self-cleaving viral 2A peptides, for example, a porcine teschovirus-1 (P2A) peptide, a Thosea asigna virus (T2A) peptide, an equine rhinitis A virus (E2A) peptide, or a foot-and-mouth disease virus (F2A) peptide. Self-cleaving 2A peptides allow expression of multiple gene products from a single construct. (See, for example, Chng et al. “Cleavage efficient 2A peptides for high level monoclonal antibody expression in CHO cells,” MAbs, 7(2): 403-412 (2015)). In some embodiments, the first and second self-cleaving peptides are the same. In some embodiments, the first and the second self-cleaving peptides are different.

C. Additional Antigen-Binding Polypeptides

In some embodiments, the modified unstimulated immune cell or modified stimulated immune cell (e.g., modified unstimulated or stimulated T cell) expresses an antigen-binding domain, a cell surface receptor ligand, or a polypeptide that binds to a tumor antigen. In some instances, the antigen-binding domain comprises an antibody that recognizes a cell surface protein or a receptor expressed on a tumor cell. In some instances, the antigen-binding domain comprises an antibody that recognizes a tumor antigen. In some instances, the antigen-binding domain comprises a full length antibody or an antigen-binding fragment thereof, a Fab, a F(ab)₂, a monospecific Fab₂, a bispecific Fab₂, a trispecific Fab₂, a single-chain variable fragment (scFv), a diabody, a triabody, a minibody, a V-NAR, or a VhH.

In some embodiments, the modified unstimulated immune cell or modified stimulated immune cell (e.g., modified unstimulated or stimulated T cell) expresses a cell surface receptor ligand. In some instances, the ligand binds to a cell surface receptor expressed on a tumor cell. In some cases, the ligand comprises a wild-type protein or a variant thereof that binds to the cell surface receptor. In some instances, the ligand comprises a full-length protein or a functional fragment thereof that binds to the cell surface receptor. In some cases, the functional fragment comprises about 90%, about 80%, about 70%, about 60%, about 50%, or about 40% in length as compared to the full length version of the protein but retains binding to the cell surface receptor. In some cases, the ligand is a de novo engineered protein that binds to the cell surface receptor. Exemplary ligands include, but are not limited to, epidermal growth factor (EGF), platelet-derived growth factor (PDGF), or Wnt3A.

In some embodiments, the modified unstimulated immune cell or modified stimulated immune cell (e.g., modified unstimulated or stimulated T cell) expresses a polypeptide that binds to a tumor antigen. In some instances, the tumor antigen is associated with a hematologic malignancy. Exemplary tumor antigens include, but are not limited to, CD19, CD20, CD22, CD33/IL3Ra, ROR1, mesothelin, c-Met, PSMA, PSCA, Folate receptor alpha, Folate receptor beta, EGFRvIII, GPC2, Tn-MUC1, GDNF family receptor alpha-4 (GFRa4), fibroblast activation protein (FAP), and IL13Ra2. In some instances, the tumor antigen comprises CD19, CD20, CD22, BCMA, CD37, Mesothelin, PSMA, PSCA, Tn-MUC1, EGFR, EGFRvIII, c-Met, HER1, HER2, CD33, CD133, GD2, GPC2, GPC3, NKG2D, KRAS, or WT1. In some instances, the polypeptide is a ligand of the tumor antigen, e.g., a full-length protein that binds to the tumor antigen, a functional fragment thereof, or a de novo engineered ligand that binds to the tumor antigen. In some instances, the polypeptide is an antibody that binds to the tumor antigen.

### IV. Compositions

Compositions of the present invention may comprise a modified unstimulated immune cell as described herein, or a modified stimulated immune cell as described herein, optionally in combination with one or more pharmaceutically or physiologically acceptably carriers, diluents, adjuvants, or excipients. Such compositions may comprise buffers such as neutral buffered saline, phosphate buffered saline and the like; carbohydrates such as glucose, mannose, sucrose, or dextrans, mannitol; proteins; polypeptides or amino acids such as glycine, antioxidants; chelating agents such as EDTA or glutathione; adjuvants (e.g., aluminum hydroxide); and preservatives. Compositions of the present invention are preferably formulated for parenteral administration (e.g., intravenous administration).

### V. Methods of Use

In some embodiments, disclosed herein is a method of treating a disease in a subject, which comprises administering to the subject a population of modified unstimulated immune cells described herein, or a population of modified stimulated immune cells described herein. In some instances, the disease is a cancer, optionally a solid tumor or a hematologic malignancy. In some instances, the modified unstimulated immune cells or the modified stimulated immune cells each expresses an antigen-binding domain that is specific for an antigen expressed by the cancer.

In some embodiments, the cancer is a solid tumor. Exemplary solid tumors include, but are not limited to, bladder cancer, bone cancer, brain cancer (e.g., glioma, glioblastoma, neuroblastoma), breast cancer, colorectal cancer, esophageal cancer, eye cancer, head and neck cancer, kidney cancer, lung cancer, melanoma, mesothelioma, ovarian cancer, pancreatic cancer, prostate cancer, or stomach cancer. In some instances, the solid tumor is brain cancer (e.g., glioma, glioblastoma, neuroblastoma), breast cancer, lung cancer, melanoma, mesothelioma, ovarian cancer, pancreatic cancer, or prostate cancer. In some instances, the solid tumor is a metastatic cancer. In some cases, the solid tumor is a relapsed or refractory solid tumor.

In some embodiments, the cancer is a hematologic malignancy. In some embodiments, the hematologic malignancy is a B-cell malignancy or a T-cell malignancy. In some embodiments, the hematologic malignancy is a lymphoma, a leukemia, or a myeloma. In some embodiments, the hematologic malignancy is a Hodgkin's lymphoma, or a non-Hodgkin's lymphoma. Exemplary hematologic malignancy include, but are not limited to, chronic lymphocytic leukemia (CLL), small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some instances, the hematologic malignancy is a metastatic hematologic malignancy. In some cases, the hematologic malignancy is a relapsed or refractory hematologic malignancy.

In some embodiments, the method of treating a disease further comprises administering to the subject an additional therapeutic agent or an additional therapy.

In some cases, an additional therapeutic agent disclosed herein comprises a chemotherapeutic agent, an immunotherapeutic agent, a targeted therapy, radiation therapy, or a combination thereof. Illustrative additional therapeutic agents include, but are not limited to, alkylating agents such as altretamine, busulfan, carboplatin, carmustine, chlorambucil, cisplatin, cyclophosphamide, dacarbazine, lomustine, melphalan, oxalaplatin, temozolomide, or thiotepa; antimetabolites such as 5-fluorouracil (5-FU), 6-mercaptopurine (6-MP), capecitabine, cytarabine, floxuridine, fludarabine, gemcitabine, hydroxyurea, methotrexate, or pemetrexed; anthracyclines such as daunorubicin, doxorubicin, epirubicin, or idarubicin; topoisomerase I inhibitors such as topotecan or irinotecan (CPT-11); topoisomerase II inhibitors such as etoposide (VP-16), teniposide, or mitoxantrone; mitotic inhibitors such as docetaxel, estramustine, ixabepilone, paclitaxel, vinblastine, vincristine, or vinorelbine; or corticosteroids such as prednisone, methylprednisolone, or dexamethasone.

In some cases, the additional therapeutic agent comprises a first-line therapy. As used herein, “first-line therapy” comprises a primary treatment for a subject with a cancer. In some instances, the cancer is a primary cancer. In other instances, the cancer is a metastatic or recurrent cancer. In some cases, the first-line therapy comprises chemotherapy. In other cases, the first-line treatment comprises radiation therapy. A skilled artisan would readily understand that different first-line treatments may be applicable to different type of cancers.

In some cases, the additional therapeutic agent comprises an immune checkpoint inhibitor. In some instances, the immune checkpoint inhibitor comprises an inhibitors such as an antibody or fragments (e.g., a monoclonal antibody, a human, humanized, or chimeric antibody) thereof, RNAi molecules, or small molecules to PD-1, PD-L1, CTLA4, PD-L2, LAG3, B7-H3, KIR, CD137, PS, TFM3, CD52, CD30, CD20, CD33, CD27, OX40, GITR, ICOS, BTLA (CD272), CD160, 2B4, LAIR1, TIGHT, LIGHT, DR3, CD226, CD2, or SLAM.

Exemplary checkpoint inhibitors include pembrolizumab, nivolumab, tremelimumab, or ipilimumab.

In some embodiments, the additional therapy comprises radiation therapy.

In some embodiments, the additional therapy comprises surgery.

### VI. Kits and Articles of Manufacture

In some embodiments, a kit or article of manufacture described herein includes one or more populations of the modified unstimulated immune cells (e.g., modified unstimulated T cells) or one or more populations of modified stimulated immune cells (e.g., modified stimulated T cells). In some instances, the kit or article of manufacture described herein further include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

A kit typically includes labels listing contents and/or instructions for use, and package inserts with instructions for use. A set of instructions will also typically be included.

### VII. Definitions

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described herein.

As used herein, the singular forms “a”, “an,” and “the” include plural referents unless the context clearly indicates otherwise. For example, the term “a cell” includes a plurality of cells, including mixtures thereof.

As used herein, the term “about” is used to indicate that a value includes the standard deviation of error for the device or method being employed to determine the value. The term “about” when used before a numerical designation, e.g., temperature, time, amount, and concentration, including range, indicates approximations which may vary by (+) or (−) 15%, 10%, 5%, 3%, 2%, or 1%.

The practice of the present disclosure will employ, unless otherwise indicated, conventional techniques of tissue culture, immunology, molecular biology, microbiology, cell biology and recombinant DNA, which are within the skill of the art. See, e.g., Green and Sambrook eds. (2012) Molecular Cloning: A Laboratory Manual, 4th edition; the series Ausubel et al. eds. (2015) Current Protocols in Molecular Biology; the series Methods in Enzymology (Academic Press, Inc., N.Y.); MacPherson et al. (2015) PCR 1: A Practical Approach (IRL Press at Oxford University Press); MacPherson et al. (1995) PCR 2: A Practical Approach; McPherson et al. (2006) PCR: The Basics (Garland Science); Harlow and Lane eds. (1999) Antibodies, A Laboratory Manual; Greenfield ed. (2014) Antibodies, A Laboratory Manual; Freshney (2010) Culture of Animal Cells: A Manual of Basic Technique, 6th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Herdewijn ed. (2005) Oligonucleotide Synthesis: Methods and Applications; Hames and Higgins eds. (1984) Transcription and Translation; Buzdin and Lukyanov ed. (2007) Nucleic Acids Hybridization: Modern Applications; Immobilized Cells and Enzymes (IRL Press (1986)); Grandi ed. (2007) In Vitro Transcription and Translation Protocols, 2nd edition; Guisan ed. (2006) Immobilization of Enzymes and Cells; Perbal (1988) A Practical Guide to Molecular Cloning, 2nd edition; Miller and Calos eds, (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Lundblad and Macdonald eds. (2010) Handbook of Biochemistry and Molecular Biology, 4th edition; and Herzenberg et al. eds (1996) Weir's Handbook of Experimental Immunology, 5th edition.

“Allogeneic” refers to any material derived from a different animal of the same species.

As used herein, the term “antibody” refers to such assemblies (e.g., intact antibody molecules, immunoadhesins, or variants thereof) which have significant known specific immunoreactive activity to an antigen of interest (e.g. a tumor associated antigen). Antibodies and immunoglobulins comprise light and heavy chains, with or without an interchain covalent linkage between them. Basic immunoglobulin structures in vertebrate systems are relatively well understood.

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

The antigen binding domain of, e.g., a chimeric antigen receptor, includes antibody variants. As used herein, the term “antibody variant” includes synthetic and engineered forms of antibodies which are altered such that they are not naturally occurring, e.g., antibodies that comprise at least two heavy chain portions but not two complete heavy chains (such as, domain deleted antibodies or minibodies); multi-specific forms of antibodies (e.g., bi-specific, tri-specific, etc.) altered to bind to two or more different antigens or to different epitopes on a single antigen); heavy chain molecules joined to scFv molecules and the like. In addition, the term “antibody variant” includes multivalent forms of antibodies (e.g., trivalent, tetravalent, etc., antibodies that bind to three, four or more copies of the same antigen.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit a desired immune response. Moreover, the skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “autologous” is meant to refer to any material derived from the same individual to which it may later to be re-introduced into the individual.

The term “chimeric antigen receptor” or “CAR,” as used herein, refers to an artificial T cell receptor that is engineered to be expressed on an immune effector cell or precursor cell thereof and specifically bind an antigen. CARs may be used in adoptive cell therapy with adoptive cell transfer. In some embodiments, adoptive cell transfer (or therapy) comprises removal of T cells from a patient, and modifying the T cells to express the receptors specific to a particular antigen. In some embodiments, the CAR has specificity to a selected target, for example, PSMA, or MUC1. CARs may also comprise an intracellular activation domain, a transmembrane domain and an extracellular domain comprising an antigen binding region.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

The term “cleavage” refers to the breakage of covalent bonds, such as in the backbone of a nucleic acid molecule or the hydrolysis of peptide bonds. Cleavage can be initiated by a variety of methods, including, but not limited to, enzymatic or chemical hydrolysis of a phosphodiester bond. Both single-stranded cleavage and double-stranded cleavage are possible. Double-stranded cleavage can occur as a result of two distinct single-stranded cleavage events. DNA cleavage can result in the production of either blunt ends or staggered ends.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

The term “non-homologous end joining” or NHEJ refers to a process in which cleaved or nicked ends of a strand of DNA are directly ligated without the need for a homologous template nucleic acid. Repair of NHEJ can lead to the addition, the deletion, substitution, or a combination thereof of one or more nucleotides at the repair site.

The term “homology-directed repair” or HDR refers to a process in which cleaved or nicked ends of a strand of DNA are repaired by insertion of a homologous template, or donor, nucleic acid. When this occurs, the original DNA sequence is replaced with the homologous template DNA. The homologous template nucleic acid can be provided by homologous sequences elsewhere in the genome (sister chromatids, homologous chromosomes, or repeated regions on the same or different chromosomes). An exogenous template nucleic acid can be introduced to obtain a specific HDR-induced change of the sequence at the target site. This allows for specific mutations or transgenes to be introduced at the cut site. The exogenous template may be a single-stranded DNA (ssDNA) template or a double-stranded DNA (dsDNA) template encoding the transgene or mutation to be introduced by HDR. In some cases, the ssDNA or dsDNA template comprises two homologous regions, for example, a 5′ end and a 3′ end, flanking a region that contains a heterologous sequence to be inserted at a target cut or insertion site.

The term “upstream” as used herein, refers to nucleic acid sequences which are found 5′ to a particular site or locus in the genome, e.g., a cleavage site catalyzed by a genome editing system. The term “downstream” as used herein, refers to nucleic acid sequences which are found 3′ to a particular site or locus in the genome.

“Effective amount” or “therapeutically effective amount” as used interchangeably herein, refer to an amount of a compound, formulation, material, pharmaceutical agent, or composition, as described herein effective to achieve a desired physiological, therapeutic, or prophylactic outcome in a subject in need thereof. Such results may include, but are not limited to an amount that when administered to a mammal, causes a detectable level of immune response compared to the immune response detected in the absence of the composition of the invention. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like. The effective amount may vary among subjects depending on the health and physical condition of the subject to be treated, the taxonomic group of the subjects to be treated, the formulation of the composition, assessment of the subject's medical condition, and other relevant factors.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

The term “epitope” as used herein is defined as a small chemical molecule on an antigen that can elicit an immune response, inducing B and/or T cell responses. An antigen can have one or more epitopes. Most antigens have many epitopes; i.e., they are multivalent. In general, an epitope is roughly about 10 amino acids and/or sugars in size. In certain exemplary embodiments, the epitope is about 4-18 amino acids, about 5-16 amino acids, about 6-14 amino acids, about 7-12 amino acids, about 10-12 amino acids, or about 8-10 amino acids. One skilled in the art understands that generally the overall three-dimensional structure, rather than the specific linear sequence of the molecule, is the main criterion of antigenic specificity and therefore distinguishes one epitope from another. Based on the present disclosure, a peptide used in the present invention can be an epitope.

As used herein, the term “exogenous” refers to any material introduced from or produced outside an organism, cell, tissue or system.

The term “expand” as used herein refers to increasing in number, as in an increase in the number of T cells. In one embodiment, the T cells that are expanded ex vivo increase in number relative to the number originally present in the culture. In another embodiment, the T cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “knockdown” as used herein refers to a decrease in gene expression of one or more genes.

The term “knockout” as used herein refers to the ablation of gene expression of one or more genes.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, e.g., a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and polymerase chain reaction, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “specificity” refers to the ability to specifically bind (e.g., immunoreact with) a given target antigen (e.g., a human target antigen). A chimeric antigen receptor may be monospecific and contain one or more binding sites which specifically bind a target or a chimeric antigen receptor may be multi-specific and contain two or more binding sites which specifically bind the same or different targets. In certain embodiments, a chimeric antigen receptor is specific for two different (e.g., non-overlapping) portions of the same target. In certain embodiments, a chimeric antigen receptor is specific for more than one target.

By the term “specifically binds,” as used herein with respect to an antibody, is meant an antibody or binding fragment thereof (e.g., scFv) which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, a chimeric antigen receptor, or a peptide with a second chemical species, to mean that the interaction is dependent upon the presence of a particular structure (e.g., an antigenic determinant or epitope) on the chemical species; for example, a chimeric antigen receptor recognizes and binds to a specific protein structure rather than to proteins generally. If an antibody is specific for epitope “A,” the presence of a molecule containing epitope A (or free, unlabeled A), in a reaction containing labeled “A” and the antibody, will reduce the amount of labeled A bound to the antibody.

The term “unstimulated,” as the term is used herein, refers to the state of an immune cell (e.g., a T cell) that has not been stimulated or induced by the binding of a stimulatory molecule (e.g., a TCR/CD3 complex) with its cognate ligand. Unstimulated T cells may also be known as “quiescent” or “naïve” T cells and have not been stimulated by means known in the art. Unstimulated cells express markers that may differentiated the cells from other cells (e.g., stimulated T cells) in a population, which include but are not limited to CD45RA and CD62L.

“Quiescent” as used herein, refers to a cell, preferably a T cell, that is in a reversible state in which it does not divide, but retains the ability to re-enter cell proliferation. Quiescent T cells are characterized by small cell size, low proliferative capacity, and low basal metabolic programs. Quiescent cells can be stimulated to divide and proliferate. Quiescent cells are a population of cells that are substantially non-proliferating. Quiescence may be measured by a number of means, including but not limited to cell proliferation assays such as BrdU assay, EdU assay, MTT cell proliferation assay, XTT cell proliferation assay, WST-1 cell proliferation assay, and measuring of markers such as Ki67 and proliferating cell nuclear antigen (PCNA).

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, clonal expansion, and differentiation into distinct subsets.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

As used herein, the term “non-expansion condition” in reference to culturing an unstimulated immune cell (e.g., a modified unstimulated immune cell) refers to culture conditions that do not stimulate the immune cell. In some embodiments, the culture media does not comprise a stimulatory molecule or stimulatory ligand that would induce a stimulatory response in the unstimulated immune cell.

As used herein, the terms “subject” and “patient” are used interchangeably. As used herein, a subject is can be a mammal, such as a non-primate (e.g., cows, pigs, horses, cats, dogs, rats, etc.) or a primate (e.g., monkey and human). In certain embodiments, the term “subject,” as used herein, refers to a vertebrate, such as a mammal. Mammals include, without limitation, humans, non-human primates, wild animals, feral animals, farm animals, sport animals, and pets. Any living organism in which an immune response can be elicited may be a subject or patient. In certain exemplary embodiments, a subject is a human.

A “target site” or “target sequence” refers to a genomic nucleic acid sequence that defines a portion of a nucleic acid to which a binding molecule may specifically bind under conditions sufficient for binding to occur.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (a) and beta (β) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “therapeutic” as used herein means a treatment and/or prophylaxis. A therapeutic effect is obtained by suppression, remission, or eradication of a disease state.

As used herein, the term “therapy” refers to any protocol, method and/or agent (e.g., a CAR-T) that can be used in the prevention, management, treatment and/or amelioration of a disease or a symptom related thereto. In some embodiments, the terms “therapies” and “therapy” refer to a biological therapy (e.g., adoptive cell therapy), supportive therapy (e.g., lymphodepleting therapy), and/or other therapies useful in the prevention, management, treatment and/or amelioration of a disease or a symptom related thereto, known to one of skill in the art such as medical personnel.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

As used herein, the terms “treat,” “treatment” and “treating” refer to the reduction or amelioration of the progression, severity, frequency and/or duration of a disease or a symptom related thereto, resulting from the administration of one or more therapies (including, but not limited to, a CAR-T therapy directed to the treatment of solid tumors). The term “treating,” as used herein, can also refer to altering the disease course of the subject being treated. Therapeutic effects of treatment include, without limitation, preventing occurrence or recurrence of disease, alleviation of symptom(s), diminishment of direct or indirect pathological consequences of the disease, decreasing the rate of disease progression, amelioration or palliation of the disease state, and remission or improved prognosis.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai virus vectors, adenovirus vectors, adeno-associated virus vectors, retrovirus vectors, lentivirus vectors, and the like.

As used herein, the term “based on” in reference to a Cas endonuclease refers to a Cas molecule having about 60%, 70%, 80%, 90%, 95%, 96%, 97%, 98%, 99%, or 100% identity compared to a reference or target Cas endonuclease of interest.

As used herein, the term “complete media” and “complete medium” refers to a cell culture media which are optimized for immune cell growth (e.g., T cell growth). In some instances, a complete media comprises proteins, inorganic salts, trace elements, vitamins, amino acids, lipids, carbohydrates, cytokines, and/or growth factors, in which the ratio of each components has been optimized for cell growth. Exemplary proteins include albumin, transferrin, fibronectin, and insulin. Exemplary carbohydrates include glucose. Exemplary inorganic salts incoude sodium, potassium, and calcium ions. Exemplary trace elements include zinc, copper, selenium, and tricarboxylic acid. Exemplary amino acids include essential amino acids such as L-glutamine (e.g., alanyl-l-glutamine or glycyl-l-glutamine); or non-essential amino acids (NEAA) such as glycine, L-alanine, L-asparagine, L-aspartic acid, L-glutamic acid, L-proline, and/or L-serine. In some embodiments, the complete media also comprises one or more of sodium bicarbonate (NaHCO₃), HEPES (4-(2-hydroxyethyl)-1-piperazine ethanesulfonic acid), phenol red, antibiotics, and/or β-mercaptoethanol. In some instances, the complete media is a serum-free media. In some instances, the complete media is a xeno-free media.

As used herein, the term “chemically defined media” refers to a cell culture media in which the compositions and concentrations of all components are known. It differs from a complete media in that the complete media may contain components, e.g., animal-derived components, in which the composition and/or concentration are not known.

In some instances, a “xeno-free” media does not contain any animal-derived (non-human) component. In some instances, a xeno-free media contains one or more human-derived components such as human serum, growth factors, and insulin.

In some embodiments, a “serum-free” media does not contain serum or plasma but may contain components derived from serum or plasma. In some instances, the “serum-free” media contains animal-derived components such as bovine serum albumin (BSA).

In some embodiment, a “minimum” media comprises the minimal necessities for growth of a target cell. In some instances, the minimum media contains inorganic salts, carbon source, and water. In some instances, supplements, cytokines, and/or proteins such as albumin (e.g., HSA) are added to the minimum media. As used herein, supplements comprise trace elements, vitamins, amino acids, lipids, carbohydrates, cytokines, growth factors, or a combination thereof.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### EXAMPLES

These examples are provided for illustrative purposes only and not to limit the scope of the claims provided herein.

### Example 1—General Vector-Free Manufacturing Method for Preparing Modified Unstimulated T Cells

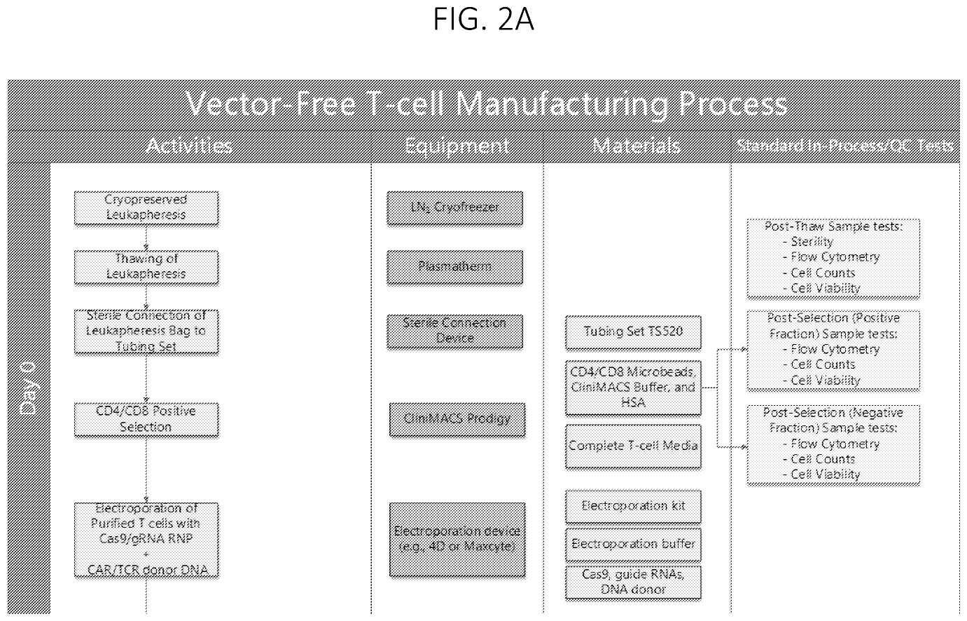

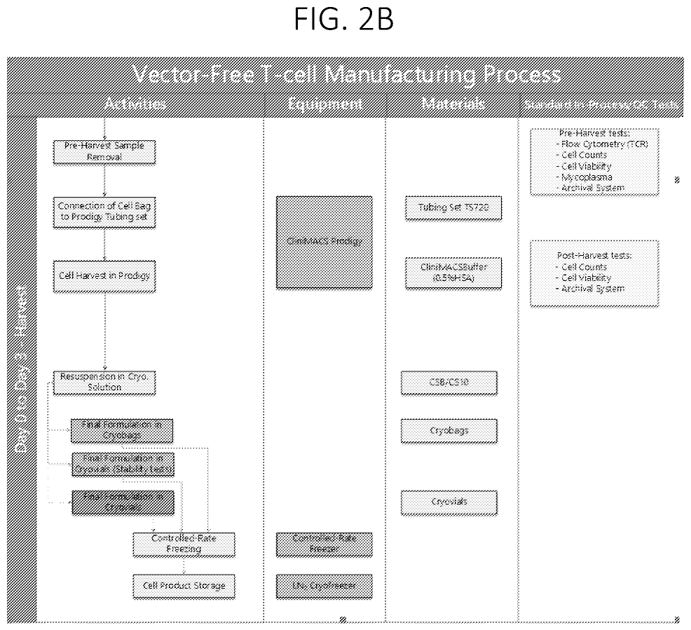

Modified unstimulated T cells were prepared based on the method as illustrated in FIG. 2.

In brief, unstimulated T cells were processed from a fresh leukapheresis sample. T-cell selection from leukapheresis was performed by a CliniMACS Prodigy based on the manufacturer's protocol and using CD4 and CD8 microbeads for positive selection of the CD4+ and CD8+ T cells. The enriched CD4+ and CD8+ T cells were then resuspended in electroporation buffer.

Cas9 endonuclease, gRNA, and a HDR template targeting the TCR alpha constant (TRAC) locus were incubated and subsequently transfected into the population of unstimulated T cells by using either a 4D Nucleofector (Lonza) or an ATx or GTx electroporator (MaxCyte). The ratio of the Cas9 endonuclease, gRNA, and the HDR template was 1:1:1.

The transfected T cells were subsequently cultured for about 72 hours prior to harvesting and frozen at −80° C.

### Example 2—Disruption of Endogenous TCR Expression in Unstimulated T Cells Using CRISPR/Cas9 with a TRAC-Targeting gRNA

CRISPR/Cas9 gene editing was performed to target the TCR alpha constant (TRAC) locus in unstimulated T cells.

Two CRISPR/Cas9 ribonucleoprotein (RNP) systems were tested: Truecut.v2 Cas9 (ThermoFisher Scientific) and SpyFi Cas9 (Aldevron). T cells that were mock electroporated with no RNP were used as a positive control. Cells were analyzed by flow cytometry for CD3 epsilon and TCR alpha/beta expression 72 hours post-electroporation with the RNP complex.

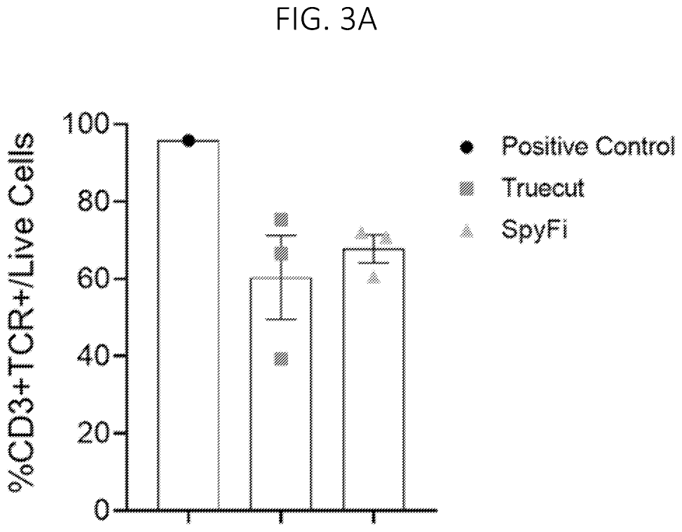

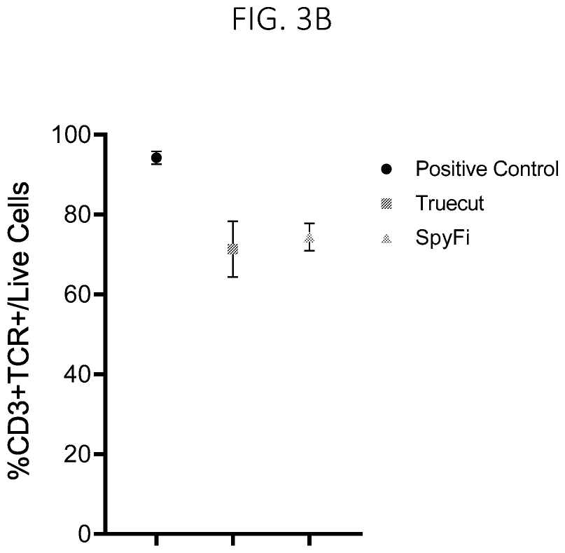

FIG. 3A and FIG. 3B illustrate that the percent of cells expressing endogenous TCR is significantly lower in the cells with either the Truecut or the SpyFi CRISPR/Cas9 RNPs as compared to the positive control cells that were not electroporated with RNP.

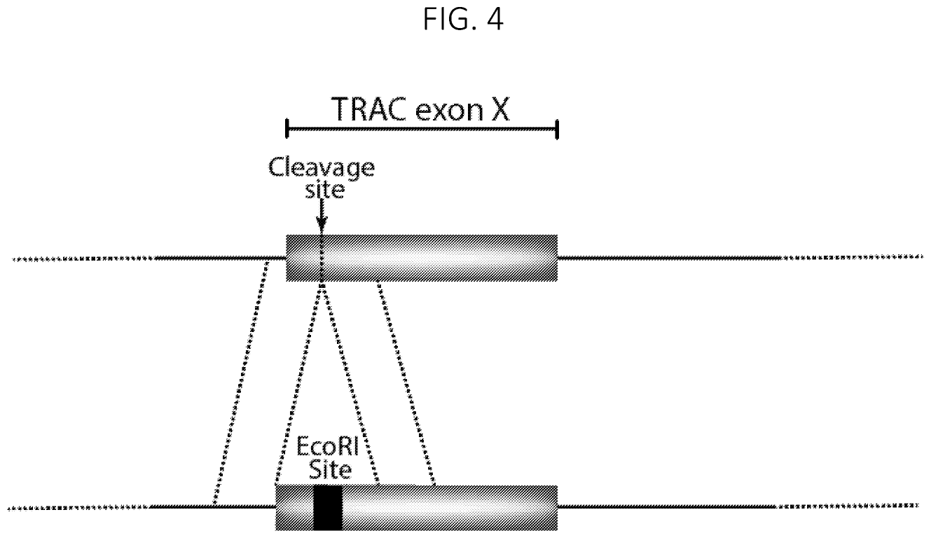

A donor DNA template was introduced to test HDR-mediated insertion into the disrupted TRAC locus. A single-stranded DNA (ssDNA) ultramer oligonucleotide and chemically modified ssDNA donors were designed with an EcoRI restriction site flanked by a 5′ homology arm, and a 3′ homology arm (FIG. 4). The homology arms were designed to have homology upstream and downstream of the CRISPR/Cas9 cut site in the TRAC locus. The ssDNA donors were introduced into the cell via electroporation. HDR-mediated insertion was tested with or without, and Primers were designed to genomic sequences upstream and downstream of the insertion, including homology arms, and the insertion sites were amplified via polymerase chain reaction (PCR).

The PCR products were then incubated with EcoRI and the resulting DNA fragments were separated and visualized using agarose electrophoresis.

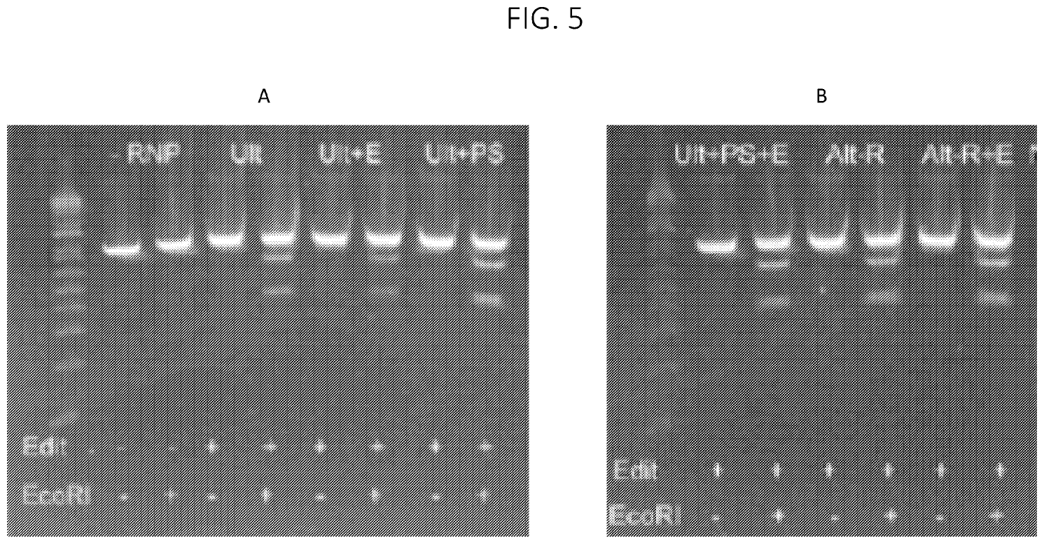

FIG. 5 illustrates the results of the EcoRI digestion. Successful insertion of the donor DNA is indicated by the lower molecular weight bands in the lanes with both CRISPR/Cas9 editing and digestion by EcoRI.

### Example 3—Knock-In Strategy to Insert a Donor DNA Encoding the NY-ESO-1 TCR into the TRAC Locus

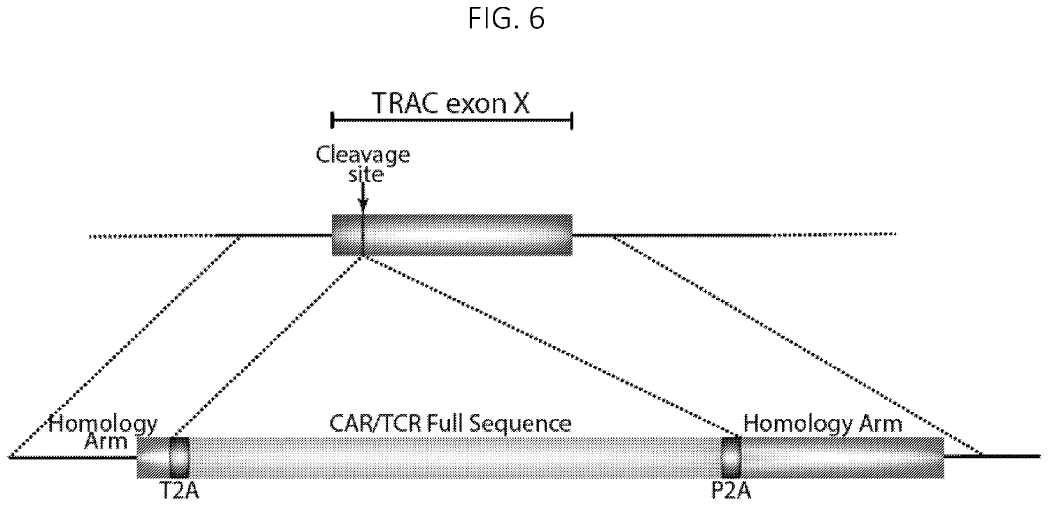

FIG. 6 illustrates the strategy for HDR-mediated insertion of an engineered TCR specific for NY-ESO-1 into the TRAC locus.

A donor DNA was designed containing the full TCRα and TCRβ (VJ region only) chains. After cleavage of the TRAC exon 1 site by CRISPR, homology arms on both sides of the payload allow for the insertion of the exogenous TCR.

The self-cleaving peptides (T2A and P2A) are removed post-translation resulting the constitutive expression of full-length TCRα and TCRβ chains of the NY-ESO-1 TCR.

### Example 4—Insertion of a Donor DNA Encoding a Target Protein into a Specific Locus in the T Cell Genome

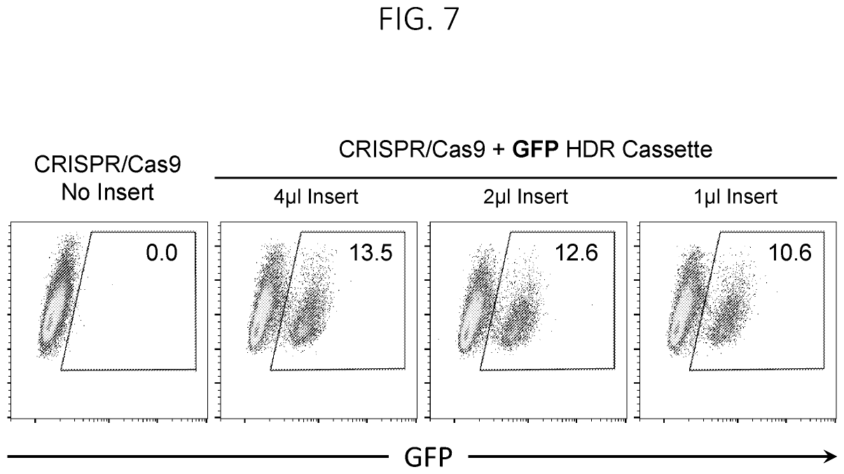

A DNA donor encoding green fluorescent protein (GFP HDR cassette) was designed and inserted into a specific targeted region within the T cell genome. As described above, the GFP gene was flanked by a 5′ homology arm and a 3′ homology arm. FIG. 7 shows the detection of GFP+ T cells at different percentages depending on the amount of DNA donor used in each reaction.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof, inclusive of the endpoints. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

1. A vector free method of preparing a population of modified unstimulated immune cells, comprising:
(a) delivering into a population of unstimulated immune cells obtained from a biological sample (i) a gene-editing nuclease, (ii) a guide RNA, and (iii) a homology-directed repair (HDR) template comprising a polynucleotide encoding an antigen-binding polypeptide, by a transfection method; and
(b) culturing the population under non-expansion conditions for about 72 hours or less,
wherein the gene-editing nuclease and the guide RNA form a complex to generate a double-stranded break at a target site within at least one unstimulated immune cell, and
wherein the HDR template facilitates HDR at the target site to generate at least one modified unstimulated immune cell within the population.

2. A vector free method of preparing a population of modified unstimulated immune cells, comprising:
(a) obtaining an enriched population of unstimulated immune cells from a biological sample;
(b) delivering into the enriched population (i) a gene-editing nuclease, (ii) a guide RNA, and (iii) a homology-directed repair (HDR) template comprising a polynucleotide encoding an antigen-binding polypeptide by a transfection method; and
(c) culturing the population under non-expansion conditions;
wherein the gene-editing nuclease and the guide RNA form a complex to generate a double-stranded break at a target site within a plurality of unstimulated immune cells within the population, and
wherein the HDR template facilitates HDR at the target site to generate a plurality of modified unstimulated immune cells, and
wherein about 10% or higher of the unstimulated immune cells within the population are modified.

3. A vector-free method of generating a population of modified unstimulated immune cells, comprising:
(a) inducing a homology-direct repair (HDR) in about 10% or higher of a population of unstimulated immune cells by:
(i) contacting the population of unstimulated immune cells with a gene-editing nuclease and a homology-directed repair (HDR) template comprising a polynucleotide encoding an antigen-binding polypeptide; and
(ii) delivering into the unstimulated immune cells the gene-editing nuclease and the HDR template by a transfection method; and
(b) culturing the unstimulated immune cells under non-expansion conditions for about 72 hours or less, thereby generating the population of modified unstimulated immune cells.

4. The method of claim 3, further comprising:
(a) contacting the population of unstimulated immune cells with a guide RNA and delivering the guide RNA into the unstimulated immune cells by a transfection method, and optionally
(b) wherein the gene-editing nuclease and optionally the guide RNA generate a double stranded break at a target site within the genome of one or more unstimulated immune cells.

5. The method of claim 3, wherein the HDR template facilitates HDR at the target site in about 10% or higher of the population of unstimulated immune cells.

6. The method of claim 3, wherein the population of unstimulated immune cells:
(a) is obtained from a biological sample; and/or
(b) comprises an enriched population of CD4+ T cells, CD8+ T cells, or a combination thereof.

7. The method of claim 1, wherein the population of unstimulated immune cells comprises about 5 million cells, 10 million cells, 20 million cells, 50 million cells, 100 million cells, 500 million cells, 1 billion cells, 2 billion cells, 3 billion cells, 4 billion cells, 5 billion cells or more.

8. The method of claim 1, wherein the transfection method:
(a) provides an efficiency of from about 10% to about 99%, from about 12% to about 99%, from about 13% to about 99%, from about 15% to about 99%, from about 20% to about 99%, from 30% to about 99%, from about 40% to about 99%, from about 50% to about 99%, from about 60% to about 99%, from about 70% to about 99%, from about 10% to about 80%, from about 12% to about 80%, from about 13% to about 80%, from about 15% to about 80%, from about 20% to about 80%, from about 30% to about 80%, from about 40% to about 80%, from about 50% to about 80%, from about 20% to about 70%, or from about 30% to about 60%; and/or
(b) provides an efficiency of about 10%, 12%, 13%, 15%, 18%, 20%, 25%, 30%, 35%, 40%, 45%, 50%, 60%, 70%, 80%, 90%, 95%, or 99%.

9. The method of claim 1, wherein the method provides a cell viability:
(a) of from about 10% to about 99%, from about 12% to about 99%, from about 13% to about 99%, from about 15% to about 99%, from about 20% to about 99%, from 30% to about 99%, from about 40% to about 99%, from about 50% to about 99%, from about 60% to about 99%, from about 70% to about 99%, from about 10% to about 80%, from about 12% to about 80%, from about 13% to about 80%, from about 15% to about 80%, from about 20% to about 80%, from about 30% to about 80%, from about 40% to about 80%, from about 50% to about 80%, from about 20% to about 70%, or from about 30% to about 60%; and/or
(b) of about 10%, about 12%, about 13%, about 15%, about 18%, about 20%, about 25%, about 30%, about 35%, about 40%, about 45%, about 50%, about 60%, about 70%, about 80%, about 90%, about 95%, or about 99%.

10. The method of claim 1, wherein:
(a) about 1 pM to about 10 mM of the HDR template is delivered into the unstimulated immune cells; and/or
(b) about 1 pM to about 10 mM of the gene-editing nuclease is delivered into the unstimulated immune cells; and/or
(c) about 1 pM to about 10 mM of the guide RNA is delivered into the unstimulated immune cells.

11. The method of claim 1, wherein:
(a) the ratio of the gene-editing nuclease to guide RNA is about 10:1, about 5:1, about 2:1, about 1:1, about 1:2, about 1:5, or about 1:10; and/or
(b) the ratio of the HDR template to the complex formed between the gene-editing nuclease and the guide RNA is about 5:1, about 2:1, about 1:1, about 1:2, or about 1:5; and/or
(c) the ratio of the HDR template to the gene-editing nuclease is about 5:1, about 2:1, 1:1, about 1:2, or about 1:5.

12. The method of claim 1, wherein the complex is a ribonucleoprotein (RNP) complex.

13. The method of claim 1, wherein the population of unstimulated immune cells:
(a) comprises unstimulated T cells, unstimulated Natural Killer (NK) cells, unstimulated natural killer T (NKT) cells, or a combination hereof; and/or
(b) comprises unstimulated T cells comprising CD4⁺ T cells, CD8⁺ T cells, CD4⁺/CD8⁺ T cells, or a combination thereof; and/or
(c) comprises an enriched population of CD4⁺ T cells, CD8⁺ T cells, or a combination thereof.

14. The method of claim 2, wherein the enriched population comprises about 90%, about 95%, about 99%, or about 100% CD4⁺ T cells, CD8⁺ T cells, or a combination thereof.

15. The method of claim 1, further comprising a step of incubating the biological sample with a plurality of CD4 and/or CD8 labeled microbeads, optionally magnetized microbeads, prior to generating the population of unstimulated immune cells.

16. The method of claim 15, wherein the step further comprises:
(a) incubating the biological sample with a solution comprising albumin, optionally human serum albumin (HSA); and/or
(b) a cell selection process to enrich the unstimulated cells in the population; and/or
(c) incubating the enriched unstimulated cells in a cell media comprising minimum media, HSA, cytokines, supplements, or a combination thereof.

17. The method of claim 1, wherein the modified unstimulated immune cells:
(a) are cultured under non-expansion conditions for about 48 hours or less, about 36 hours or less, about 24 hours or less, or about 18 hours or less; and/or
(b) are cultured under non-expansion conditions for about 18 hours, about 24 hours, about 36 hours, about 48 hours, or about 72 hours; and/or
(c) are further resuspended in a cryopreservant solution and cryo-frozen.

18. The method of claim 1, wherein the transfection method comprises:
(a) electroporation; or
(b) a cell squeezing method.

19. The method of claim 1, wherein the antigen-binding polypeptide:
(a) comprises an antigen-binding domain;
(b) comprises a chimeric antigen receptor (CAR);
(c) comprises a cell surface receptor ligand; or
(d) comprises a T-cell receptor (TCR); and/or
(e) binds to a tumor antigen.

20. The method of claim 19, wherein:
(a) the antigen-binding domain comprises a full length antibody or an antigen-binding fragment thereof, a Fab, a F(ab)₂, a monospecific Fab₂, a bispecific Fab₂, a trispecific Fab₂, a single-chain variable fragment (scFv), a diabody, a triabody, a minibody, a V-NAR, or a VhH; and/or
(b) the CAR comprises the antigen-binding domain, a transmembrane domain, and an intracellular domain, and optionally wherein the CAR comprising a hinge region.

21. The method of claim 20, wherein:
(a) the transmembrane domain is selected from an artificial hydrophobic sequence, a transmembrane domain of a type I transmembrane protein, an alpha, beta, or zeta chain of a T cell receptor, CD28, CD3 epsilon, CD45, CD4, CD5, CD8, CD9, CD16, CD22, CD33, CD37, CD64, CD80, CD86, OX40 (CD134), 4-1BB (CD137), ICOS (CD278), CD154, and a transmembrane domain derived from a killer immunoglobulin-like receptor (KIR); and/or
(b) the intracellular domain comprises a costimulatory signaling domain and an intracellular signaling domain; and/or
(c) the intracellular domain comprises one or more of a costimulatory domain of a protein selected from the group consisting of proteins in the TNFR superfamily, CD27, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS (CD278), NKG2C, B7-H3 (CD276), and an intracellular domain derived from a killer immunoglobulin-like receptor (KIR), or a variant thereof.

22. The method of claim 21, wherein:
(a) the costimulatory domain comprises a 4-1BB (CD137) costimulatory domain; and/or
(b) the intracellular signaling domain comprises an intracellular domain selected from the group consisting of cytoplasmic signaling domains of a human CD3 zeta chain (CD3ζ), FcγRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof, and/or
(c) the intracellular signaling domain comprises a 4-1BB costimulatory domain and a human CD3 zeta chain (CD3ζ) cytoplasmic signaling domain.

23. The method of claim 22, wherein the cytoplasmic signaling domain comprises a human CD3 zeta chain (CD3ζ).

24. The method of claim 20, wherein the tumor antigen:
(a) is associated with a hematologic malignancy; and/or
(b) is selected from CD19, CD20, CD22, and CD33/IL3Ra.; and/or
(c) is associated with a solid tumor; and/or
(d) is selected from ROR1, mesothelin, c-Met, PSMA, PSCA, Folate receptor alpha, Folate receptor beta, EGFRvIII, GPC2, TnMUC1, GDNF family receptor alpha-4 (GFRa4), fibroblast activation protein (FAP), and IL13Ra2.

25. The method of claim 19, wherein the TCR:
(a) comprises a TCR alpha chain and a TCR beta chain; and/or
(b) is selected from a wild-type TCR, a high affinity TCR, and a chimeric TCR.

26. The method of claim 1, wherein the HDR template:
(a) further comprises a 5′ homology arm upstream of the polynucleotide; and/or
(b) further comprises a 3′ homology arm downstream of the polynucleotide; and/or
(c) is a double-stranded DNA template; and/or
(d) is a double-stranded DNA template, wherein the HDR template is from about 2 kilo-base pairs (kb) to about 5 kb, from about 2.3 kb to about 5 kb, from about 3 kb to about 5 kb, from about 3 kb to about 4 kb, from about 2 kb to about 4 kb, from about 2.3 kb to about 4 kb, from about 2 kb to about 3 kb, from about 2.3 kb to about 3 kb, or from about 4 kb to about 5 kb in length; and/or
(e) is delivered by electroporation.

27. The method of claim 26, wherein the 5′ homology arm:
(a) is adjacent to the polynucleotide; and/or
(b) is homologous to a genomic region 5′ of the target site; and/or
(c) is from about 50 nucleotides to about 500 nucleotides, from about 50 nucleotides to about 400 nucleotides, from about 50 to about 300 nucleotides, from about 50 nucleotides to about 200 nucleotides, from about 50 nucleotides to about 150 nucleotides, from about 100 nucleotides to about 500 nucleotides, from about 100 nucleotides to about 400 nucleotides, from about 100 nucleotides to about 300 nucleotides, from about 100 nucleotides to about 200 nucleotides, from about 200 nucleotides to about 500 nucleotides, from about 200 nucleotides to about 400 nucleotides, from about 200 nucleotides to about 300 nucleotides, from about 300 nucleotides to about 500 nucleotides, or from about 300 nucleotides to about 400 nucleotides in length; and/or
(d) is about 50, about 100, about 150, about 200, about 250, about 300, about 350, about 400, about 450, or about 500 nucleotides in length.

28. The method of claim 27, wherein the 3′ homology arm:
(a) is adjacent to the polynucleotide; and/or
(b) is homologous to a genomic region 3′ of the target site; and/or
(c) is from about 50 nucleotides to about 500 nucleotides, from about 50 nucleotides to about 400 nucleotides, from about 50 to about 300 nucleotides, from about 50 nucleotides to about 200 nucleotides, from about 50 nucleotides to about 150 nucleotides, from about 100 nucleotides to about 500 nucleotides, from about 100 nucleotides to about 400 nucleotides, from about 100 nucleotides to about 300 nucleotides, from about 100 nucleotides to about 200 nucleotides, from about 200 nucleotides to about 500 nucleotides, from about 200 nucleotides to about 400 nucleotides, from about 200 nucleotides to about 300 nucleotides, from about 300 nucleotides to about 500 nucleotides, or from about 300 nucleotides to about 400 nucleotides in length; and/or
(d) is about 50, about 100, about 150, about 200, about 250, about 300, about 350, about 400, about 450, or about 500 nucleotides in length.

29. The method of claim 1, wherein the target site is in the TRAC locus, and optionally exon 1 of the TRAC locus.

30. The method of claim 1, wherein the gene-editing nuclease comprises:
(a) a Cas nuclease; and/or
(b) a zinc finger nuclease; and/or
(c) a transcription activator-like effector nuclease (TALEN).

31. The method of claim 30, wherein the Cas nuclease:
(a) is Cas9, optionally SpCas9 or SaCas9; and/or
(b) and the guide RNA assembles into a complex prior to delivering into the unstimulated immune cells; and/or
(c) and the guide RNA assembles into a complex after delivering into the unstimulated immune cells.

32. The method of claim 1, further comprising delivering one or more additional guide RNA into the unstimulated immune cells.

33. The method of claim 1, wherein the biological sample:
(a) is a blood sample; and/or
(b) is a blood sample, wherein the blood sample is a whole blood sample, a peripheral blood mononuclear cell (PBMC) sample, or an apheresis sample; and/or
(c) is a blood sample, wherein the blood sample is an apheresis sample which is cryopreserved; and/or
(d) is a blood sample, wherein the blood sample is an apheresis sample which is fresh.

34. The method of claim 1, further comprising:
(a) stimulating the modified unstimulated immune cells to generate a population of modified stimulated immune cells, and optionally
(b) expanding the population of modified stimulated immune cells.

35. The method of claim 34, wherein the population of modified stimulated immune cells is cultured under expansion conditions for about 1 day, about 2 days, about 3 days, about 4 days, about 5 days, about 6 days, about 7 days, about 8 days, about 9 days, about 10 days, about 11 days, about 12 days, or more.

36. The method according to claim 1, wherein the immune cells comprise T cells, natural killer cells, natural killer T cells, macrophages, monocytes, B cells, hematopoietic stem cells, or combinations thereof.

37. A population of modified unstimulated immune cells generated by a method according to claim 1.

38. A population of modified stimulated immune cells generated by a method according to claim 1.

39. A composition comprising a population of modified unstimulated immune cells generated by a method according to claim 1, or a population of modified stimulated immune cells generated by a method according to claim 1; optionally comprising a pharmaceutically acceptable excipient.

40. A method of treating a disease in a subject in need thereof, comprising administering the population of modified unstimulated immune cells to the subject; or administering the population of modified stimulated immune cells to the subject, according to claim 1.

41. The method of claim 40, wherein the subject has a cancer.

42. The method of claim 41, wherein the cancer is a solid tumor.

43. The method of claim 42, wherein the cancer is a hematologic malignancy.

44. The method of claim 40, wherein the antigen-binding domain is specific for an antigen expressed by the cancer.

45. The method of claim 40, wherein the biological sample is autologous to the subject.

46. The method of claim 40, wherein the biological sample is allogeneic to the subject.

47. The method of claim 40, wherein the subject is a human.

48. A kit comprising a population of modified unstimulated immune cells of claim 37.
